(19) United States
(12) Patent Application Publication
(10) Pub. No.: US 2002/0016293 A1
(43) Pub. Date: Feb. 7, 2002

(54) Flavopiridol drug combinations and methods with reduced side effects

(76) Inventors: Mark J. Ratain, Chicago, IL (US); Federico Innocenti, Chicago, IL (US); Lalitha Iyer, Chicago, IL (US)

Correspondence Address: Gina N. Shishima, Fulbright & Jaworski L.L.P., Suite 2400, 600 Congress Avenue, Austin, TX, 78701, US

(21) Appl. No.: 09/835,082

(22) Filed: Apr. 12, 2001

Related U.S. Application Data
(63) Continuation-in-part of application No. 09/553,829, filed on Apr. 21, 2000.

(51) Int. Cl. A61K 38/00; A61K 31/56; A61K 31/57; A61K 31/497

(52) U.S. Cl. 514/9; 514/171; 514/105; 514/252.13

(57) ABSTRACT

This invention provides methods, formulations and kits to reduce the toxicity of flavopiridol and analogs thereof. Disclosed are therapeutics and treatment methods employing such drugs in combination with agents that increase conjugative enzyme activity or glucuronosyltransferase activity, and agents that decrease biliary transport protein activity, such as cyclosporine A, the resultant effects of which are to decrease the significant side effects previously associated with treatment using these drugs. The invention also characterizes specific isoforms of glucuronyltransferase enzymes involved in glucuronidation of flavopiridols and their analogs.

The present application is a continuation-in-part of co-pending U.S. patent application Ser. No. 09/553,829, filed Apr. 21, 2000. The entire text of each of the above-referenced disclosures is specifically incorporated by reference herein without disclaimer.

The government owns rights in the present invention pursuant to grant number U01CA63187-01 from the National Cancer Institute.

### BACKGROUND OF THE INVENTION

1. Field of the Invention

The present invention relates generally to the fields of cancer therapy. More particularly, it concerns new treatment methods and compositions for optimizing the treatment of cancer patients with flavopiridol.

2. Description of Related Art

Flavopiridol (NSC 649890) is a novel semisynthetic flavone with direct inhibition of cyclin-dependent kinases, anti-proliferative effects in vitro in a p53-independent manner, and activity in colon and prostate carcinoma animal models. Flavopiridol inhibits cell cycle progression in either G1 or G2 and is capable of killing noncycling as well as cycling cells (Bible et al., 1996). The drug is currently undergoing clinical evaluation, although dose-limiting toxicity results in moderate to severe diarrhea. Preclinical data on flavopiridol metabolism indicates that the drug undergoes hepatic glucuronidation.

Flavopiridol exhibits a unique pattern of differential growth inhibitory activity at concentrations below 200 nM, as well as cytostatic properties in xenografted human tumors (Czech, et al. 1995; Sedlacek, et al. 1996). Investigations into its mechanism of action reveal that flavopiridol is an inhibitor of several cyclin-dependent kinases (CDK; Carlson, et al. 1996; Kaur, et al. 1992; Losiewicz, et al. 1994; Worland, et al. 1993). These mediators of cell cycle progression are often deregulated in transformed cells (Sherr, et al. 1996), and have been suggested to be excellent targets for anti-neoplastic drug development (Sausville, et al. 1999). Flavopiridol can be cytostatic or cytotoxic to neoplastic cells, depending on the concentration and the duration of exposure (Bible, et al. 1997; Chien, et al. 1999; Shapiro, et al. 1999; Schrump, et al. 1998). The preclinical studies suggested that prolonged exposure is important for maximizing flavopiridol activity (Drees, et al. 1997), and thus continuous infusion schedules have been most extensively explored in human trials (Thomas, et al. 1997; Senderowicz, et al. 1998).

Flavopiridol's metabolism in humans has not been characterized in detail. An in vivo animal study indicated that the glucuronidation of flavopiridol is the major mechanism of flavopiridol transformation (Jager, et al. 1998). We demonstrated the ability of human liver microsomes to convert flavopiridol to its corresponding glucuronide (Ramirez, et al. 1998), providing evidence for existence of this metabolic pathway in humans as well. Pharmacology studies during phase I trials focused only on the disposition of the parent drug, and showed that plasma concentrations necessary to inhibit in vitro CDK activity and cell proliferation can be easily achieved (Thomas, et al. 1997; Senderowicz, et al. 1998). The dose-limiting toxicity for flavopiridol administered as a 72 h-continuous infusion is secretory diarrhea (Thomas, et al. 1997; Senderowicz, et al. 1998). The occurrence of intestinal toxicity may limit the utility of flavopiridol, especially since induction of apoptosis in preclinical systems requires concentrations above that achievable at the maximum-tolerated dose. Interestingly, the maximum-tolerated dose of flavopiridol can be increased from 50 to 78 mg/m²/day when patients are treated with prophylactic antidiarrheal treatment with cholestyramine and loperamide (Thomas, et al. 1997). The amelioration of diarrhea by the use of cholestyramine is consistent with the finding that cholestyramine binds flavopiridol preventing its toxic action on the gut mucosa (Thomas, et al. 1997). Moreover, preliminary results in vitro showed that flavopiridol is a modulator of the intestinal epithelial chloride secretion (Stadler, et al. 2000).

One of the flavopiridols, (FLAVO; NSC 649890) is a synthetic flavone derivative currently undergoing Phase II clinical development as an anticancer agent. Its mechanism of action is by direct inhibition of cyclin-dependent kinases (Carlson et al., 1996; Senderowicz et al., 1998). The major dose-limiting toxicity of FLAVO is secretory diarrhea (Senderowicz et al., 1998; Stadler et al., 2000). FLAVO is metabolized in both rats and humans via glucuronidation (Jager et al., 1998; Innocenti et al., 2000). As described above, upon administration of FLAVO to metastatic renal cancer patients high inter-individual variation was observed (coefficient of variation 72-99%) in the capacity to glucuronidate FLAVO. Glucuronidation of FLAVO appeared to be bimodal, and subjects displaying extensive glucuronidation seemed to be protected from suffering diarrhea (Innocenti et al., 2000). The specific enzyme(s) involved in the glucuronidation of FLAVO are unknown.

Glucuronidation by uridine diphosphate glucuronosyltransferase (UGT) enzymes is a major drug metabolic pathway in humans. This conjugation reaction results in the formation of water-soluble products that are readily excreted into the bile or urine (Green et al., 1994; Radominska-Pandya et al., 1999). The UGT enzymes are broadly classified into 2 distinct families, UGT1 and UGT2, based on similarities between their primary amino acid sequences (Radominska-Pandya et al., 1999). There are nine functional human UGT1A isoforms (UGT1A1 and UGT1A3-10), and five of them are expressed in hepatic tissue (UGT1A1, UGT1A3, UGT1A4, UGT1A6, and UGT1A9; Strassburg et al., 1998; Radominska-Pandya et al., 1999). The UGT2 family is divided into subfamilies 2A, 2B, and 2C (Radominska-Pandya et al., 1999), with UGT2B isoforms being the most abundant. Six human UGT2B isoforms (UGT2B4, UGT2B7, UGT2B10, UGT2B11, UGT2B15, and UGT2B17) have been identified (Radominska-Pandya et al., 1999).

As FLAVO glucuronidation in cancer patients represents a crucial metabolic pathway, there is an acute need to investigate the metabolic pathways involved and determine the enzyme(s) responsible for FLAVO glucuronidation.

### SUMMARY OF THE INVENTION

The instant invention addresses the issue of reducing or overcoming the dose limiting toxicity of flavopiridol. The instant invention provides methods and composition for reducing allaying or eliminating symptoms associated with flavopiridol treatment. The invention further provides methods for determining individuals at an increased risks for exhibiting similar symptoms in response to administration of the drug.

In certain embodiments, the invention thus provides methods for reducing flavopiridol toxicity comprising administering flavopiridol or an analog thereof in combination with an effective amount of one or more second agents that increase conjugative enzyme activity or that decreases biliary, or bile canaliculi, transport protein activity.

“Effective amounts” in certain circumstances are those amounts effective to reproducibly increase conjugative enzyme activity, or to decrease biliary transport protein activity, in comparison to their normal levels. Preferably, compounds that achieve significant appropriate changes in activity will be used. If desired, a battery of compounds may be screened in vitro to identify second agents for use in the present invention. Here, significant increases in conjugative enzyme activity, e.g., as measured using a glucuronosyltransferase assay, are represented by increases of at least about 30%-40%, and most preferably, by increases of at least about 50%, with higher values of course being possible. Glucuronosyltransferase assays are well known in the art and may be conducted in vitro or in vivo.

Significant decreases in activity, when using a cellular or biliary transport assay, are represented by decreases of at least about 30%-40%, and most preferably, of at least about 50%, with more significant decreases also being possible. Biliary transport protein binding and inhibition assays are well known in the art, generally in the context of reversing multi-drug resistance (MDR). Assays may be conducted as described in, for example Ichikawa-Haraguchi et al. (1993; incorporated herein by reference). One potentially effective transport assay is that described by Thalhammer et al. (1994; incorporated herein by reference) that measures the p-glycoprotein-mediated transport of the cationic dye, acridine orange, across the bile canaliculi. Thalhammer et al. (1994) showed that this activity was inhibited by cyclosporine A and verapamil. Therefore, if a candidate substance exhibited inhibition in this type of study, it would likely be a candidate compound for use in the present invention.

However, quantitative in vitro testing is not a requirement of the invention as it is generally envisioned that the second agents will often be selected on the basis of their known properties or by structural and/or functional comparison to those agents disclosed herein. Therefore, the effective amounts will often be those amounts proposed to be safe for administration to animals in another context, for example, as disclosed herein. As the invention arises in part from the inventors' discovery of certain metabolic and physiological events, and the inventors' surprising combination of elements, there is considerable information available on the use and doses of second agents alone, which information may now be employed with the present invention.

So long as a dose of second agent that does not exceed previously quoted toxicity levels is not required, the effective amounts of the second agents may simply be defined as those amounts effective to reduce the side-effects or toxicity of one or more first flavopiridol drugs when administered to an animal in combination with the first flavopiridol drug(s). This is easily determined by monitoring the animal or patient and measuring those physical and biochemical parameters of health and disease that are indicative of the success of a given treatment. Such methods are routine in animal testing and clinical practice. Preferably, compounds that show a significant reduction in toxicity will be used, as will be determinable by the ordinary clinician.

In certain embodiments, the doses of flavopiridol drugs and other analogues, used in the present invention will often be less that those used in the prior art. Indeed, this is one advantage of the invention as it provides for a smaller dose to be given in order to achieve the same beneficial anti-cancer or other therapeutic results.

In other embodiments, the doses of flavopiridol drugs administered may be about the same as those currently used in the art. In such cases, using the flavopiridol compound in combination with a second agent that reduces biliary excretion of the active species or metabolite will result in increased bioavailability of the active component. This may be used, for example, in patients that have advanced disease or that have proven resistant to lower doses of flavopiridols. Higher flavopiridol levels may also be used, so long as the second agents are provided in amounts to prevent significant toxicity or untoward effects in the recipient animal or patient.

In any event, as the invention provides for reducing the toxicity of flavopiridol drugs and for increasing the bioavailability of flavopiridol drugs, it will be apparent that this invention provides for more variability in the doses of flavopiridol species than previous methods. The attending physician may thus optimize treatment to the individual patient, effectively accounting for the variations in disease heterogeneity that were previously a problem.

The present invention also provides the opportunity for effective therapy without using a combination of chemotherapeutic agents. Although the use of additional drugs and chemotherapeutic combinations is contemplated in certain aspects of the invention, largely on a patient-by-patient basis, in many situations an advantage of this invention will be that other chemotherapeutics will not be necessary to achieve a significant response. This is beneficial as it will reduce overall toxicity and also remove the possibility of adverse, or even fatal, drug interactions.

In further embodiments, the flavopiridol drug or drugs could be administered in combination with both one or more second agents that increase conjugative enzyme activity and one or more other second agents (or a so-called “third agents”) that inhibit biliary transport activity. This would give the added advantage of reducing the biliary excretion of the active flavopiridol species by intervening in two different metabolic pathways.

It will be understood that the term “conjugative enzyme” as used herein, refers to enzymes that modify active flavopiridol species.

In terms of second agents that increase conjugative enzyme activity, such agents may increase phase I conjugative enzyme activity, but will preferably increases phase II conjugative enzyme activity. As used herein, the term “conjugative enzyme activity” is used to describe those enzymes that increase the water-solubility of metabolites, via conjugation, so that the resultant conjugate may be more readily excreted. The term “phase II conjugative enzyme” is also used to refer to enzymes that may be more commonly known as phase II enzymes.

Examples of phase I oxidative enzymes include the cytochrome P450 enzymes. Examples of phase II conjugative enzymes including the glucuronosyltransferase enzymes, glutathione S-transferase (GST), N-acetyl transferase, and even quinone reductase (QR; Prochaska & Fernandes, 1992). As glucuronidation of flavopiridol has been specifically observed in patients, compounds that increase glucuronosyltransferase enzyme activity are currently preferred. Specific examples of glucuronosyltransferase enzymes of particular relevance to the instant invention include uridine 5′diphosphate glucoronyltansferase (UGT)1A1, UGT1A3, UGT1A4, UGT1A6, UGT1A8, UGT1A10, UGT2B4, UGT2B7, UGT2B15 and UGT2B17. Other specific examples of glucuronyltransferase enzymes that may be administered as the second agent of the method include but are not limited to uridine 5′diphosphate glucoronyltansferase (UGT)1A1, UGT1A3, UGT1A4, UGT1A7, or UGT1A9. Among these the glucuronyltransferase enzyme UGT1A9 is particularly preferred. When a UGT enzyme or UGT-encoding nucleic acid is described in the present specification it is contemplated that that description also applies to any isoform of UGT.

Second agents that decrease or inhibit biliary transport and excretion are exemplified by agents that reduce transport of compounds into the bile, and even those that reduce bile flow, i.e., cholestatic agents. Inhibition of transport is generally achieved by inhibiting any membrane transport protein, or protein complex, that is present in the bile canaliculi and that functions to transport flavopiridol analogues. It is contemplated that exemplary proteins with transport activity will be members of the ABC protein family.

To identify second agents capable of inhibiting biliary transport activity, an initial screen may be conducted on the basis of biliary transport protein binding, or inhibition of photoaffinity labeling of biliary transport proteins (Akiyama et al., 1988), followed by studies to confirm inhibitory activity. Inhibitory activity may be confirmed by competing for transport of labeled compounds in vitro, inhibiting the transport of labeled compounds in vitro, or even reversal of drug resistance in cells in vitro. A number of substrates for various biliary transport proteins are currently known to one of skill in the art. It will be appreciated that based upon this knowledge, inhibitors of flavopiridol transport are readily determinable. Of course, it will be further appreciated that animal testing and pre-clinical studies showing reduced flavopiridol toxicity are the preferred means for optimizing the invention.

Virtually any method may be employed to increase the activity of a conjugative enzyme, such as glucuronosyltransferase, including increasing the levels of the enzyme, increasing the activity of a fixed amount of the enzyme, removing an inhibitor of the enzyme, and the like. For example, methods to increase the levels of a conjugative enzyme, such as glucuronosyltransferase, include increasing its transcription, translation or stability. Methods to increase the activity of such conjugative enzymes include administering specific or general activators of a given enzyme or enzyme family; removing any specific or general inhibitors, and such like. Known inducers of glucuronosyltransferase generally act by enhancing the de novo synthesis of the enzyme (Bock et al., 1978).

As to the inhibition of biliary transporter activity a variety of methods are again available. For example, decreasing the levels of the enzyme or transporter, decreasing the activity of a fixed amount of the enzyme or transporter, removing an activator or enhancer of the enzyme or transporter, and the like. Methods to decrease the levels of biliary transporter include, for example, decreasing its transcription, translation or stability. Methods to decrease the activity of the transporter include administering specific or general inhibitors of the transporter or transporter family or removing any specific or general activators.

As the present invention provides for increasing the amount of a conjugative enzyme, such as glucuronosyltransferase, and decreasing the amount of a transporter useful “second agents” also include recombinant vectors and constructs. For example, administering a recombinant form of a glucuronosyltransferase enzyme, or an antisense DNA construct that is complementary to a biliary transport protein's nucleic acid sequence, is envisioned. Second agent recombinant vectors are those that comprise a sequence region encoding a conjugative enzyme or an antisense version of a biliary transport protein, where the vectors are capable of expressing the sequence region in the type of mammalian that is to be treated.

Generally, increasing the activity or the amount of a conjugative enzyme using chemical agents will be preferred over molecular biological. Compounds that may be used as second agents to increase conjugative enzyme activity include those compounds that have been shown to be, or are believed to be, inducers of cytochrome P450 enzymes. These include, for example, cyclophosphamide; ifosphamide; dilantin (also known as phenytoin); disulfiram (also known as Antabuse); rifampin; clonazepam and clotrimazole (Lubet et al, 1992).

Barbiturates, such as phenobarbital, which are often used as anti-convulsants, may also be employed as second agents to activate conjugative enzymes. 3-methylcholanthrene may also be used to induce glucuronosyltransferases, where it induces different isoforms of the enzyme to phenobarbital (Rajaonarison et al., 1993; Burchell & Coughtrie, 1989).

Another group of compounds that may be used to increase conjugative enzyme activity are the retinoic acids, such as all trans retinoic acid, 9-cis retinoic acid and 13-cis retinoic acid. The anti-AIDS drug zidovudine (also known as AZT) may also be used in limited circumstances, mostly in combination with rifampin, as described by Burger et al. (1993). Any of the many corticosteroids, could be employed, with dexamethasone as a particular example. Oral contraceptives, such as those described herein, form another possibility for use as second agents.

Compounds particularly contemplated for use as second, anti-toxicity agents in the context of increasing drug conjugation are those compounds that are known to be, or are believed to be, capable of promoting glucuronidation. These include L-buthionine-S,R-sulfoximine (BSO; Manning & Franklin, 1990); and anti-oxidants, such as butylated hydroxyanisole (BHA; tert-butyl-4-hydroxyanisole; Ansher et al., 1983).

Within the group of compounds that promote glucuronidation, two groups of compounds are currently preferred. First, the dithiolethiones (also known as dithiolthiones, Ansher et al., 1983), examples of which include 3H-1,2,-dithiole-3-thione; 3H-1,2,-dithiole-3-one; 1,3-dithiole-2-thione; [1,2]dithiolo[4,3-c]-1,2-dithiole-3,6-dithione; 4-methyl-3H-1,2-dithiole-3-thione; 5-methyl-3H-1,2-dithiole-3-thione; 4,5-dimethyl-3H-1,2-dithiole-3-thione; 4-ethyl-3H-1,2-dithiole-3-thione; 5-ethyl-3H-1,2-dithiole-3-thione; 5-tert-butyl-3H-1,2-dithiole-thione; 3-thioxo-3H-1,2-dithiole-4-carboxylic acid; 3-thioxo-3H-1,2-dithiole-5-carboxylic acid; 3-thioxo-3H-1,2-dithiole-4-carboxamide; 3-thioxo-3H-1,2-dithiole-5-carboxamide; 4-phenyl-3H-1,2-dithiole-3-thione; 5-phenyl-3-thione; 4-methyl-5-phenyl-3H-1,2-dithiole-3-thione-S-oxide; 4,5,6,7-tetrahydrobenzo-3H-1,2-dithole-3-thione; 5,6-dihydrocyclopenta-1,2-dithiole-3 (4H)-thione; 4-methyl-5-pyrazinyl-3H-1,2-dithiole-3-thione (oltipraz); 4-methyl-5-pyrazinyl-3H-1,2-dithiole-3-thione-S-oxide; 7-methyl-6,8-bis(methylthio)pyrrolo-[1,2-a]-pyrazine; 5-(4-methoxyphenol)-3H-1,2-dithiole-3-thione; 5-(4-methoxyphenol)-4-methyl-3H-1,2-dithiole-3-thione (Egner et al., 1994). Oltipraz (RP-35972; 4-methyl-5(2-pyrazinyl)-3H-1,2-dithiole-3-thione; available from Rhone-Poulenc) is particularly preferred, and has been shown to act upon acetaminophen (Davies & Schnell, 1991).

Further preferred compounds that are capable of promoting glucuronidation include those known as aryloxycarboxylic acids, arylcarboxylic acids, chlorophenoxycarboxylic acids or fibric a acids, which compounds often function as hypolipidemic compounds (Boiteux-Antoine et al., 1989). Suitable aryloxycarboxylic and arylcarboxylic acids include clofibrate, ciprofibrate, fenofibrate, bezafibrate, gemfibrazol, tiadenol, probucol and the active compound 2-phenylpropionic acid (Magdalou et al., 1993; Fournel et al., 1985; Boiteux-Antoine et al., 1989). The term “clofibric acid” itself includes, 4′-chlorophenoxyacetic, 4′-chlorophenoxypropionic and 4′-chlorophenoxyisobutyric acids. All such fibric acids have been shown to be effective at inducing glucuronidation and are thus contemplated for use in the present invention.

Currently, preferred compounds for use in increasing conjugative enzyme activity are phenobarbital, Oltipraz, all-trans retinoic acid, phenytoin, dexamethasone, rifampin, and fibric acids, such as clofibrate. Oltipraz, all-trans retinoic acid, rifampin and phenobarbital are currently particularly preferred.

For reducing biliary transport, decreasing the activity of biliary transport proteins, rather than decreasing the number of such proteins, will generally be preferred. Compounds that may be used as second agents to inhibit biliary transporters, include those compounds that have been shown to be, or are believed to be, general or specific inhibitors of membrane transport components. For example, as well as inhibitors that deactivate or modify the biliary transporters (i.e., non-competitive inhibitors), agents that compete transporter binding sites and render the transporter less available for the flavopiridol drug in question are contemplated (i.e., competitive inhibitors).

A currently particularly preferred group of second agents are immunosuppressants, such as cyclosporines, cyclosporine derivatives, and even cephalosporins, such as cefoperazone. Even non-immunosuppressive cyclosporine derivatives have biliary transporter-blocking capabilities. One such example is SDZ PSC 833 (valspodar), which is more effective than cyclosporine A (Boesch et al., 1991; Pourtier-Manzanedo et al., 1992; Boesch & Loor, 1994; Zacherl et al., 1994). SDZ 28-446 is another cyclosporine A analogue that may be employed (Pourtier-Manzanedo et al., 1992).

Cyclosporine A, C, G and H will generally be used, with cyclosporine A being particularly preferred. However, the cyclosporine D analogue 3′-Keto-cyclosporine D (Bell et al., 1994) may also be used. Staurosporine and staurosporine derivatives, particularly NA-382, may also be employed (Miyamoto et al., 1992a; 1993).

Further examples of biliary transporter inhibitors are calcium channel blockers. One particularly useful group of compounds are the dihydropyridine analogues (Kamiwatari et al., 1989), and their clinical counterparts. Certain examples are verapamil, dex verapamil and their analogues (Ohi et al., 1992; Doige et al., 1992; Inoue et al., 1993; Hunter et al., 1993; Thalhammer et al., 1994; Muller et al., 1994; Bear, 1994; Boesch & Loor, 1994).

Other useful calcium channel blockers include tiapamil and tiapamil analogues, such as 1993RO-11-2933 (Campain et al., 1993); nifedipine and nifedipine analogues (Wilson et al., 1991; Doige et al., 1992; Hunter et al., 1993); diltiazem (Morris et al., 1991); nicardipine (Niwa et al., 1992); prenylamine; nimodipine; nisoldipine and nitrendipine.

Further examples of second agents that inhibit biliary transporters are calmodulin antagonists. This group of agents includes thioxanthenes, phenothiazines and flupenthixols, such as cis-flupenthixol, trans-flupenthixol and clorpenthixol (Ford et al., 1990; Hait et al., 1993). Other calmodulin antagonists include clomipramine, fluphenazine, chlorpromazine, triflupromazine, trifluoperazine, prochlorperazine and thioridazine.

Still further examples of second agents that inhibit the biliary transporters are anti-neoplastic agents. Although the invention encompasses a wide variety of other agents, the anti-neoplastic compounds may also be employed. Examples of these are vincristine (Shirai et al., 1994; Friche et al., 1993); vinblastine (Bear, 1994; McKinney & Hosford, 1993); actinomycin D (McKinney & Hosford, 1993); colchicine (Bear, 1994; McKinney & Hosford, 1993; Doige et al., 1992); etoposide; daunomycin (Bear, 1994); daunorubicin (Muller et al., 1994); doxorubicin (Mechetner & Roninson, 1992) and analogues, such as 14-O-hemiesters of doxorubicin; taxotere (Hunter et al., 1993); taxol (Mechetner & Roninson, 1992); and tamoxifen (Trump et al., 1992).

Yet further examples of second agents that inhibit the biliary transporters are cationic compounds, such as reserpine; dipyridamole (DPM; Suzuki, 1990; Tatsuta et al, 1991); chloroquine, quinacrine, propranolol, cepharanthine and other compounds described by Akiyama et al. (1988).

A diverse group of other agents have been shown to interact with and biliary transport. For example, certain steroids (Chin et al., 1992), including pregnenolone, progesterone and metabolites (Ichikawa-Haraguchi et al., 1993; Gruol et al., 1994; Doige et al., 1992); RU 486; and 21-aminosteroid derivatives, lazaroids and tirilazad (Abraham et al., 1993). Dexamethasone is also contemplated for use in this aspect (Miller et al., 1991). Certain bile acids, such as taurochenodeoxycholate, glycochenodeoxycholate, taurolithocholate and ursodeoxycholate (Mazzanti et al., 1994) may also be used. Other agents that may be employed include anthracycline analogues such as DNR, N,N-dibenzyl-DNR and N-benzyladriamycin-14-valerate (AD-198; Friche et al., 1993); terfenadine (Seldane; Hait et al., 1993); certain dihydropyridine analogues (Suzuki, 1990; Kiue et al., 1990; Kamiwatari et al., 1989); ivermectin (Schinkel et al., 1994); quinidine (Akiyama et al., 1988); reduced folates (Suzuki et al, 1998); genistein (Jager et al, 1998); probenecid (Overboch et al, 1988) ritonavir (Zhang et al., 2000), and interferon (Savas, et al. 1999)

Antibodies that binds to external epitopes of biliary transport may also be used as second agents to achieve inhibition. Monoclonal antibodies (MAbs) will generally be preferred. Many such antibodies are known, as exemplified by MAb C219 (Miyamoto et al., 1992b) and MAbs JSB-1 and C-219 (Miller et al., 1991). The MAb UIC2 (Mechetner & Roninson, 1992), and others developed by the Schinkel group, such as HYB-241, 7G4 and 4E3, may also be used (Schinkel et al., 1993). The MAb MRK16 and the mouse-human chimeric version MH162 are preferred agents (Hamada et al., 1990), as is the mouse-human chimeric antibody, MH171 (Ariyoshi et al., 1992) and the MAb UIC2 (Mechetner & Roninson, 1992). MRK16, MH171 and UIC2 have been safely used in animals. Further examples include αMRP3-(Kool, 1999); MRP1-QCRL-1 and QCRL-3 (Filipits, 1999). Other useful monoclonal antibodies may also be obtained or prepared, so long as the MAb generally exhibits binding affinity for external epitopes of biliary transporters.

The biliary transport protein or proteins responsible for flavopiridol transport have yet to be elucidated. It is nevertheless surmised, that the transporter of flavopiridol is a member of the ABC protein family. The ABC protein family includes over 30 human proteins. (Klein, et al. 1999). Of particular relevance to biliary transport are the members of MDR/TAP and MRP/CTFR subfamilies. Exemplary proteins of the MDR/TAP subfamily include p-glycoprotein and MDR3,while exemplary proteins of the MRP/CTFR include MRP1 and cMOAT. While p-glycoprotein is the best characterized biliary transport protein, MDR3, BSEP, MRP1 and MRP2 (cMOAT; as well as, potentially, MRP3, MRP5, and MRP6) are all important mediators of drug transport (Klein, 1999).

Other members of the ABC protein family contemplated useful in context of the present invention include, but are not limited to, the ABC subfamilies such as, ABC1 (also called subfamily A and includes members identified by the symbols ABCA1 (also named variously as ABC1, TGD, HDLDT1, CERP), ABCA2, ABCA3 (also named variously as ABC-C, EST111653), ABCA4 (also named variously as ABCR, RP-19 ABCA10, FFM, STGD1, STGD), ABCA5, ABCA6, ABCA7, ABCA8, ABCA9, ABCA10, ABCA11, ABCA12, ABCA13, ABCA14); MDR/TAP (also called subfamily B and includes members identified by the symbols ABCB1 (also named variously as PGY1, MDR1, PGP, GP170), ABCB2 (also named variously as TAP1, PSF1, RING4, ABC17, APT1, D6S114E), ABCB3 (also named variously as TAP2, PSF2, RING11, ABC18, D6S17E), ABCB4 (also named variously as PGY3, MDR2, MDR3, ABC21), ABCB5, ABCB6, ABCB7, ABCB8, ABCB9 (also named variously as TAPL), ABCB10, ABCB11 (also named variously as BSEP, SPGP, PFIC2, PGY4, ABC16); CFTR/MRP (also called subfamily C and includes members identified by the symbols ABCC1 (also named variously as MRP1, MRP), ABCC2 (also named variously as CMOAT), ABCC3 (also named variously as MRP3), ABCC4 (also named variously as MRP4), ABCC5 (also named variously as MRP5), ABCC6 (also named variously as MRP6), ABCC7 (also named variously as CFTR), ABCC8 (also named variously as SUR1), ABCC9 (also named variously as SUR2), ABCC10, ABCC11, ABCC12, ABCC13); ALD (also called subfamily D and includes members identified by the symbols ABCD1, ABCD2, ABCD3, ABCD4); OABP (also called subfamily E and includes members identified by the symbols ABCE1); GCN20 (also called subfamily F and includes members identified by the symbols ABCF1, ABCF2, ABCF3); White (also called subfamily G and includes members identified by the symbols ABCG1 (also named variously as ABC8, White), ABCG2 (also named variously as BCRP1, MXR1, ABCP), ABCG4, ABCG5, ABCG8). See also http://www.med.rug.nl/mdl/humanabc.htm (incorporated herein by reference) for details and links to other ABC proteins and nucleic acids.

The use of such flavopiridol drugs has been previously limited by significant gastrointestinal toxicities, particularly manifested as moderate to severe diarrhea. By modulating flavopiridol drug toxicity, the present invention thus also provides improved methods for treating cancers, leukemias, parasitic infections and other diseases and disorders, as desired.

The treatment methods generally comprise administering to an animal with cancer, including a human patient, a therapeutically effective combination of one or more flavopiridol drugs and one or more second agents that reduce flavopiridol toxicity by reducing excretion of the active flavopiridol species through the bile, as exemplified by second agents that increase conjugative enzyme activity and/or that inhibit biliary transport activity. The second agent(s) may be any of those listed above, and their functional equivalents.

“Therapeutically effective amounts” are those amounts effective to produce beneficial results in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

In some cases, pre-clinical testing in animals with disease may not be necessary where both the flavopiridol drug and the second agent have been previously approved for human treatment. In any event, all that is required to determine or optimize a therapeutically effective amount for human treatment is to administer the first drug(s) in combination with an amount of one or more selected second agents and to monitor the patient to determine whether a benefit to the patient results. Preferably, one would use an amount that resulted in a significant benefit to the patient, as assessed by a significant reduction in flavopiridol toxicity or any increase in the anti-tumor (or anti-parasitic) response. Optimal doses of the second agents may thus be readily identified following the general starting ranges, such as those found in the scientific literature and those detailed herein.

Animals and patients may also be treated with the flavopiridol drug or drugs in combination with two or more second agents, with at least one agent being from the different classes described above. Here, the agents may also be described as a second agent and a third agent. This has the benefit of acting at two distinct points in the flavopiridol excretion pathways and may result in further improved or even synergistic effects.

In treatment methods, the first flavopiridol drug or drugs may be administered to the animal or patient prior to administering the second agent(s), or the first drug(s) and the second agent(s) may be administered simultaneously. A single composition that comprises both the first drug(s) and one or more second agents may be employed for simultaneously administration, or distinct compositions that include only one of the formulations could be used. Where one or more third agents are used, a combined therapeutic cocktail may be prepared and administered if desired.

Using distinct compositions is generally preferred where the number of second agents is relatively low, as this can provide for more control of the individual doses. However, a cocktail may be preferred where the total number of components is larger, to minimize the discomfort to the animal or patient due to repeated administration.

It is currently preferred that the second agent be administered to the animal or patient prior to the flavopiridol drug(s) in order to “prime” the system. Delivery of the second agent prior to the flavopiridol drug and continued delivery of the second agent throughout the flavopiridol delivery period is one currently preferred treatment method. “Delivery” in these contexts preferably means continuous infusion.

Various other delivery methods may also be used, as desired by the attending physician. It will likely be convenient to employ standard delivery methods, such as parenteral administration, including continuous infusion and intravenous, intramuscular and subcutaneous injections. However, other methods, such as oral delivery may be employed, depending on the second agent used to reduce the toxicity or enhance the bioavailability of the first flavopiridol drug(s).

Also provided are new compositions and formulations, including pharmacologically acceptable formulations, that comprise one or more first flavopiridols in combination with one or more second agents that increase conjugative enzyme activity or that decrease biliary transport protein activity. Such compositions may include the first flavopiridol drug or drugs in combination with Oltipraz, clofibrate, ciprofibrate, fenofibrate, bezafibrate, gemfibrazol, tiadenol, probucol, phenobarbital, dilantin, clonazepam, clotrimazole, buthionine sulfoximine (BSO), cyclophosphamide, ifosphamide, a retinoic acid, a corticosteroid, an oral contraceptive, rifampin or disulfiram (Antabuse); and will preferably include flavopiridol in combination with phenobarbital, Oltipraz, all-trans retinoic acid, phenytoin, dexamethasone, rifampin or clofibrate.

The compositions may also include one or more first flavopiridol drugs in combination with a cyclosporine or staurosporine, particularly, cyclosporine A, SDZ 280 446, 3′-Keto-cyclosporin D, cefoperazone, staurosporine, SDZ PSC 833 (valspodar), NA-382, dihydropyridine analogue, verapamil, dex verapamil, tiapamil, nifedipine, diltiazem, nicardipine, nisoldipine, nimodipine, nitrendipine, trans-flupenthixol, cis-flupenthixol, clorpenthixol, fluphenazine, chlorpromazine, triflupromazine, trifluoperazine, prochlorperazine, thioridazine, progesterone, a progesterone metabolite, pregnenolone, RU 486, tirilazad, reserpine, dipyridamole, chloroquine, propranolol, terfenadine, ivermectin, quinidine [(3)H]2,4-Dinitrophenyl-S-glutathione (DNP-SG), [(3)H]17beta-estradiol 17-beta-D-glucuronide, dipyridamole (E(2)17betaG), 7-chloro-4-nitrobenz-2-oxa-1,3-diazole, buthionine sulphoximine, MK751, leukotriene C4 (LTC4), bromosulfophthalein (BSP), enalapril, CRC 220, taurocholate (TCA), N-acetylcysteine and cysteine, [3H]Temocaprilat, estradiol-17beta-D-glucuronide, dibromosulphophthalein, genistein, probenecid, indomethacin, sulindac, tolmetin, acemetacin, zomepirac, mefenamic acid and ritonavir.

The compositions may also advantageously include the first flavopiridol drug or drugs in combination with one or more second agents selected from the group that increases conjugative enzyme activity and one or more second or third agents selected from the group that decreases biliary transport protein activity.

The terms “pharmacologically or pharmaceutically acceptable”, as used herein, refer to compositions that do not produce significant toxicity, detrimental side effects, or other untoward reactions, when given to an animal or patient. In that flavopiridols prior to the present invention, were known to suffer from certain toxic limitations, it will be understood that “pharmaceutically acceptable” compositions may still have certain harmful effects when given to an animal. “Pharmaceutically acceptable” in the present context thus refers to other components, such as diluents, binders and the like, which should be selected from the pharmaceutically acceptable products available or developed using the same general guidelines.

Therapeutic kits comprising flavopiridol drugs and one or more second or third agents form another aspect of the invention. Such kits will generally contain, in suitable container means, a pharmaceutical formulation of the flavopiridol drug(s), a pharmaceutical formulation of one or more second agents that increase conjugative enzyme activity or that decrease biliary transport protein activity. Multiple agents with different specificities may be employed. The kit may have a single container means with all the drugs and agents disposed therein or may have two, three or multiple distinct container means, one for each compound or group of similar-acting compounds.

The components of the kit may be provided as liquid solution(s), or as dried powder(s). When the components are provided in a liquid solution, the liquid solution is an aqueous solution, with a sterile aqueous solution being particularly preferred. When reagents or components are provided as a dry powder, the powder can be reconstituted by the addition of a suitable solvent. It is envisioned that the solvent may also be provided in another container means.

The components of the kit may be provided in one or more fluid or syringeable compositions. In which case, the container means may itself be an intravenous delivery bag, a syringe, pipette, eye dropper, or other such like apparatus, from which the formulation may be infused or injected into an animal, applied to a diseased area of the body, or even applied to and mixed with the other components of the kit.

The container means of the kit will generally be at least one intravenous delivery fluid bag, vial, test tube, flask, bottle, syringe or other suitable container into which the drugs and/or agents may be placed, and preferably, suitably allocated. The kits of the present invention will also typically include a means for containing the vials in close confinement for commercial sale, such as, e.g., injection or blow-molded plastic containers into which the desired vials are retained.

Irrespective of the number or type of containers, the kits of the invention may also comprise, or be packaged with, an instrument for assisting with the infusion, injection or administration of the ultimate composition to an animal. Such an instrument may be a syringe, one or more delivery tubes, or even an eye dropper or a measuring spoon, or any such medically approved delivery vehicle.

The invention also provides method for predicting the degree of flavopiridol toxicity that may arise in a patient. One such method generally comprises determining the glucuronidation capacity of the patient, wherein a decreased glucuronidation capacity, in comparison to normal levels, would be indicative of a patient at risk of developing drug toxicity, if a drug such as flavopiridol were to be given alone.

Another such method generally comprises determining the biliary transport capacity of the patient, wherein an increased biliary transport capacity, in comparison to normal levels, would be indicative of a patient at risk of developing drug toxicity, if a flavopiridol-like drug were to be administered alone.

Certain of the diagnostic methods may employ “genotyping”, i.e., assaying for genetic polymorphisms in enzymes involved in the metabolism of flavopiridols. Here, the glucuronidation or biliary transport capacity of the patient is determined by means of determining the amount of DNA or RNA encoding a glucuronosyltransferase enzyme or a biliary transport protein. The execution of such molecular biological methods is well known in the art, and includes, for example, Southern and Northern blotting performed by contacting nucleic acids from a biological sample of the patient with a DNA (or RNA) segment that encodes a mammalian glucuronosyltransferase enzyme or a biliary transport protein. This is done under conditions effective to allow hybridization of substantially complementary nucleic acids, and the hybridized nucleic acid complexes thus formed are later detected using a detectable label, such as a radiolabel.

In preferred embodiments, the diagnostic methods will generally be “phenotypic” in nature, for example, wherein the glucuronidation capacity of a patient is determined by administering a glucuronidatable substrate, such as acetaminophen, diflunisal or morphine, to the patient and then determining the degree of glucuronidation of the substrate, e.g., by detecting the substrate-glucuronide conjugate by HPLC.

The invention also provides methods for evaluating the risk of flavopiridol-induced toxicity in a patient comprising evaluating a UGT1A9-encoding nucleic acid of the patient for a polymorphism. In some aspects the method comprises identifying a patient at risk for flavopiridol-induced toxicity. In these embodiments, the method further comprises identifying a polymorphism in a UGT1A9-encoding nucleic acid of the patient. The polymorphism may results in either a decreased level of UGT1A9 activity in the patient or may result in a decreased level of UGT1A9 expression in the patient. It is contemplated that these methods may also be used in the context of any other UGT isoform.

The invention also contemplates methods for reducing the toxicity of flavopiridol or an analog thereof in a cancer patient comprising a) identifying a polymorphism in a UGT1A9 gene in a sample from the patient, wherein the polymorphism contributes to reduced expression or activity of the UGT1A9 gene product; and b) administering to the patient a second agent effective to reduce excretion of an active flavopiridol species through the bile.

The invention also provides methods for evaluating the risk of flavopiridol-induced toxicity in a patient comprising evaluating a ABC-encoding nucleic acid of the patient for a polymorphism. In some embodiments this comprises identifying a patient at risk for flavopiridol-induced toxicity. In some aspects, the method comprises identifying a polymorphism in a ABC-encoding nucleic acid of the patient. In specific aspects the polymorphism may result in a decreased level of ABC activity in the patient or a decreased level of ABC expression in the patient. In such embodiments, the ABC-encoding nucleic acid may encode any ABC protein such as but not limited to ABCA1 (ABC1, TGD, HDLDT1, CERP), ABCA2, ABCA3 (ABC-C, EST111653), ABCA4 (ABCR, RP-19 ABC10, FFM, STGD1, STGD), ABCA5, ABCA6, ABCA7, ABCA8, ABCA9, ABCA10, ABCA11, ABCA12, ABCA13, ABCA14, ABCB1 (PGY1, MDR1, PGP, GP170), ABCB2 (TAP1, PSF1, RING4, ABC17, APT1, D6S114E), ABCB3 (TAP2, PSF2, RING11, ABC18, D6S17E), ABCB4 (PGY3, MDR2, MDR3, ABC21), ABCB5, ABCB6, ABCB7, ABCB8, ABCB9 (TAPL), ABCB10, ABCB1 (BSEP, SPGP, PFIC2, PGY4, ABC16), ABCC1 (MRP1, MRP), ABCC2 (CMOAT), ABCC3 (MRP3), ABCC4 (MRP4), ABCC5 (MRP5), ABCC6 (MRP6), ABCC7 (CFTR), ABCC8 (SUR1), ABCC9 (SUR2), ABCC10, ABCC11, ABCC12, ABCC13), ABCD1, ABCD2, ABCD3, ABCD4), ABCE1, ABCF1, ABCF2, ABCF3, ABCG1 (ABC8, White), ABCG2 (BCRP1, MXR1, ABCP), ABCG4, ABCG5, or ABCG8. Of these ABC-encoding nucleic acids encodes BCRP 1 are preferred.

The invention also provides methods for reducing the toxicity of flavopiridol or an analog thereof in a cancer patient comprising a) identifying a polymorphism in a ABC gene in a sample from the patient, wherein the polymorphism contributes to reduced expression or activity of the ABC gene product; and b) administering to the patient a second agent effective to reduce excretion of an active flavopiridol species through the bile.

The invention also concerns determining whether a genotype correlates with a phenotype affecting or involved in flavopiridol toxicity. In some embodiments, a biliary transport protein, such as BCRP1 or another ABC protein, is evaluated for phenotypic differences that may result from genotype. Thus, in some embodiments, a nucleic acid encoding a biliary transport protein is evaluated for a polymorphism before or after the biliary transport protein is evaluated for activity. From this a correlation between genotype and phenotype can be made, which will allow a subject to be evaluated for risk of flavopiridol toxicity based on the genotype of a particular biliary transport protein. In other embodiments, known polymorphisms in a biliary transport protein are characterized to determine whether a correlation between genotype and flavopiridol toxicity exists. If a correlation is identified, the polymorphism can be the basis for identifying a patient at risk (or not at risk, depending on the correlation) for flavopiridol toxicity. Furthermore, this correlation can also be the basis for determining treatment regimens.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Scatterplots of flavopiridol (A), flavopiridol glucuronide (B) plasma levels, and metabolic ratios between flavopiridol glucuronide and flavopiridol (C) at 23, 47, and 71 h into the infusion. Horizontal lines represent the median value.

FIG. 2. Frequency distribution histogram of metabolic ratios between flavopiridol glucuronide and flavopiridol measured at 71 h.

FIG. 3. Scatterplots of metabolic ratios between flavopiridol glucuronide and flavopiridol measured at 71 h versus diarrhea grade.

FIG. 4. Chemical structure of FLAVO.

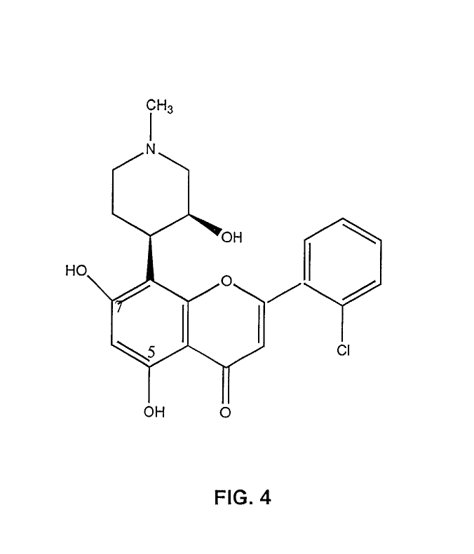

FIGS. 5A and 5B. HPLC chromatograms obtained after in vitro glucuronidation of FLAVO (0.5 mM) in liver microsomes (3 mg/ml) from (FIG. 5A) normal humans, and (FIG. 5B) a CN-1 patient.

FIG. 6. Inter-individual variability in in vitro glucuronidation of FLAVO. All data represent the mean of3 determinations±S.D. Range=0.11-1.03 FLAVO-G/flavone height ratio; mean±S.D.=0.53±0.25; coefficient of variation=47%; 9-fold variability.

FIG. 7. Contribution of UGT isoforms to the formation of FLAVO-G.

### DESCRIPTION OF ILLUSTRATIVE EMBODIMENTS

The present invention relates methods and composition for overcoming the dose limiting toxicity of the anti-cancer drug, flavopiridol and its analogues. The inventors have determined that flavopiridol is transformed to its glucuronide in the liver and excreted in the bile. Preferred embodiments of the present invention therefore relate methods and compositions of combining second agents to either increase flavopiridol catabolism or decrease biliary transport in order to reduce dose limiting side effects.

The liver is largely responsible for the metabolism of absorbed dietary flavonoids, which undergo biliary excretion after conjugation with glucuronic acid or sulfate. Glucuronidation is generally considered an inactivating metabolic pathway and provides an important means of elimination for many clinically-used drugs (Miners, et al. 1991). Conjugation with glucuronic acid by UDP-glucuronosyltransferase (UGT) usually renders the parent drug less active and more easily excreted from the body (Meech, et al. 1998). For extensively glucuronidated drugs, altered glucuronidation activity in the liver is likely to have important consequences for their clinical use (Miners et al. 1991). Two different flavopiridol glucuronides containing only one glucuronic acid in position 5 or 7 of the flavonoid core were found in rat bile (Jager, et al. 1998). Our assay method quantified all the existing O-glucuronidated metabolites of flavopiridol in patient plasma, providing a measure of overall systemic glucuronidation of flavopiridol. The present data indicate that flavopiridol is transformed to its glucuronide in the liver and excreted in the bile. The liver is largely responsible for the metabolism of absorbed dietary flavonoids, which undergo biliary excretion after conjugation with glucuronic acid or sulfate (Di Carlo, et al. 1999).

Since glucuronidation appears to be polymorphic and potentially typeable, the present inventors propose that the first step will be to determine which patients are “poor glucuronidators” and potentially at higher risk of flavopiridol induced diarrhea. The importance of a possible polymorphic metabolism of flavopiridol stems from the clinical relevance of flavopiridol glucuronidation in patients. The glucuronidating phenotype is reflective inversely of the extent of biliary excretion of flavopiridol and hence of the risk of developing diarrhea. Diarrhea was present in 91% of poor glucuronidators of flavopiridol, while 73% of extensive glucuronidators did not develop diarrhea. Patients with low metabolic ratios developed diarrhea presumably because of the exposure of the intestine to toxic levels of flavopiridol. High metabolic ratios seem to protect patients from the occurrence of diarrhea, raising the possibility that flavopiridol glucuronide does not maintain the toxicological properties of its parent compound. Similar findings were observed for SN-38, the active metabolite of irinotecan. Patients with relatively higher biliary indexes, the product of the relative AUC ratio of SN-38 to its inactive glucuronide and the total irinotecan AUC, were at increased risk of diarrhea (Gupta, et al. 1994). Considerable inter-patient variability in metabolic ratios was observed and might contribute to the heterogeneity in flavopiridol pharmacokinetics recently described by Senderowicz et al. (Senderowicz, et al. 1998). This variability in glucuronidation of flavopiridol is mainly responsible for differential accumulation of flavopiridol in the intestine of patients, and might have a major impact on the outcome of cancer therapy with flavopiridol.

The distribution of the glucuronidating phenotypes among patients is apparently bimodal, indicating the presence of a genetic polymorphism of flavopiridol glucuronidation. Drug-metabolizing enzyme genes are clearly among the modifying factors that affect enhanced risk of toxicity to drugs (Nebert, et al. 1999). Anticancer agents are detoxified by metabolizing enzymes usually showing genetic variations (Iyer, 1999). The knowledge of a pharmacokinetic polymorphism is particularly important in oncology since cytotoxic agents have a narrow therapeutic index and are administered at the maximum tolerated doses (lyer, et al. 1998). Such genetic polymorphisms have played a crucial role in drug-related toxicity in cancer chemotherapy, leading to either life-threatening toxicities or unpredictable variability in response and toxicity among patients (Gupta, et al. 1994; Diasio, et al. 1989; Ratain, et al. 1991; Lennard, et al. 1992). UGT2B variants might account for the apparent polymorphic metabolism of flavopiridol. Mutated UGT2B4, UGT2B7, and UGT2B15 enzymes have been recently described (Coffman, et al. 1198; Levesque, et al. 1997; Levesque, et al. 1999), but their functional role in vivo is still unknown.

The inventors propose that a correlation between glucuronidator status and flavopiridol toxicity exists and that it is thus very important to induce this phase II enzyme. Induction of phase II enzymes has been of major interest to investigators working in the chemoprevention field, because of the importance of these enzymes in carcinogen detoxification. One agent under investigation as an inducer of these enzyme systems is oltipraz, a dithiolthione. Oltipraz has been demonstrated to be an inducer of GST and UDP-glucuronosyltransferase, and reduces acetaminophen toxicity in a rodent model (Egner et al., 1994). Therefore oltipraz is considered as a potential agent with which to induce glucuronidation prior to flavopiridol treatment, and monitored with acetaminophen phenotyping. Such a strategy is contemplated to significantly enhance the therapeutic index of flavopiridol.

Since gastrointestinal toxicity was related to excessive amounts of flavopiridol in the bile that drained into the gut, the inventors realized that another approach to reduce toxicity of flavopiridols would be to reduce transport into the bile. The inventors thus envision that compounds that effect biliary transport could be important in regulating flavopiridol toxicity. It is specifically contemplated that the biliary transporter of flavopiridol will be a member of the ABC protein family.

ATP-binding cassette (ABC) proteins are an ancient class of membrane transporters, that have become specialized in uptake and secretion, intracellular transport, cell detoxification and signaling and translocate highly diverse compounds across cell membranes, such as ions, amphiphiles, sugars, peptides and proteins. Overexpression of ABC proteins, specifically multidrug resistance proteins, has been linked to drug resistance in cancer (Lomri, et al. 1996). The proteins consist of transmembrane domains and nucleotide binding domains. The transmembrane domains of ABC proteins generally consist of six transmembrane helices with the minimum number required for function being two. The functional units of an ABC protein consist of a hydrophobic domain of six membrane spanning segments and a hydrophilic cytoplasmic domain which is able to bind ATP. Three 20-45 amino acid sequence motifs in the nucleotide binding folds (NBFs) are highly conserved among ABC transporters. As, ABC proteins are active pumps, the nucleotide binding domains facilitate the ATP dependent transport of the substrate across the membrane. General and specific inhibitors of ABC protein transport are well known in the art (Klein, 1999). Compounds are thus readily determinable that would be applicable in the context of the instant invention for inhibiting the relevant form of drug transport.

Flavopiridol and other anticancer drugs are transported out by some of the ABC transporter proteins leading to drug-resistance. For example, cell lines overexpressing MRP1 have been reported to show higher drug-resistance to flavopiridol (Hooijberg et al., 1999). In another example, Robey et al., 2001, have shown that BCRP1 is involved in similar drug resistance in breast cancer cell lines.

Polymorphisms in some ABC transporter genes have been recently identified. For example see Ito et al., 2001; Kerb et al., 2001; Perdu and Germain, 2001; Ken-Ichi et al., 2001; Hoffmeyer et al., 2000; Decleves et al., 2000; Cascorbi et al., 2001; Rund et al, 1999, all incorporated herein by reference.

Pharmaceutical Compositions

Aqueous compositions of the present invention will have an effective amount of flavopiridol and an effective amount of a compound (second agent) that increases conjugative enzyme activity, as represented by a compound that increases the activity of the phase II conjugative enzyme, glucuronosyltransferase or that decreases biliary transport. Such compositions will generally be dissolved or dispersed in a pharmaceutically acceptable carrier or aqueous medium.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants and the like.

A. Parenteral Administration

The active compounds will often be formulated for parenteral administration, e.g., formulated for injection via the intravenous, intramuscular, sub-cutaneous, or even intraperitoneal routes. The preparation of an aqueous composition that contains flavopiridol and a second agent as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in cremes and lotions. These forms may be used for treating skin-associated diseases, such as various sarcomas.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

B. Oral Administration

In certain embodiments, active compounds may be administered orally. This is contemplated for agents which are generally resistant, or have been rendered resistant, to proteolysis by digestive enzymes. Such compounds are contemplated to include all those compounds, or drugs, that are available in tablet form from the manufacturer and derivatives and analogues thereof.

For oral administration, the active compounds may be administered, for example, with an inert diluent or with an assimilable edible carrier, or they may be enclosed in hard or soft shell gelatin capsule, or compressed into tablets, or incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of the unit. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Upon formulation, the compounds will be administered in a manner compatible with the dosage formulation and in such amount as is therapeutically effective. The formulations are easily administered in a variety of dosage forms, such as those described below in specific examples.

C. Liposomes

In a particular embodiment, liposomal formulations are contemplated. Liposomal encapsulation of pharmaceutical agents prolongs their half-lives when compared to conventional drug delivery systems. Because larger quantities can be protectively packaged, this allow the opportunity for dose-intensity of agents so delivered to cells. This would be particularly attractive in the chemotherapy of cervical cancer if there were mechanisms to specifically enhance the cellular targeting of such liposomes to these cells.

“Liposome” is a generic term encompassing a variety of single and multilamellar lipid vehicles formed by the generation of enclosed lipid bilayers. Phospholipids are used for preparing the liposomes according to the present invention and can carry a net positive charge, a net negative charge or are neutral. Dicetyl phosphate can be employed to confer a negative charge on the liposomes, and stearylamine can be used to confer a positive charge on the liposomes. Liposomes are characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are cationic lipid-nucleic acid complexes, such as lipofectamine-nucleic acid complexes

Catabolic Therapeutics

A. Olitipraz

Oltipraz (RP-35972; 4-methyl-5(2-pyrazinyl)-3H-1,2-dithiole-3-thione; available from Rhone-Poulenc) is one agent that is preferred for use with the present invention. It has been shown to exert chemoprotective effects of against carbon tetrachloride and acetaminophen toxicity (Ansher et al., 1983; Davies & Schnell, 1991; Egner et al., 1994). Therefore oltipraz is considered as a potential agent with which to induce glucuronidation prior to, or with, flavopiridol treatment. Doses such as those described by Ansher et al., 1983; Davies & Schnell, 1991; and Egner et al., 1994; each incorporated herein by reference, may be employed.

B. Aryloxycarboxyllic Acids and Fibric Acids

1. Clofibrate

Clofibrate is propanoic acid, 2-(4-chlorophenoxy)-2-methyl-,ethyl ester; Atromid-S and is available from Ayers.

It can be prepared by condensing phenol with ethyl 2-chloro-2-methylpropionate in the presence of a suitable dehydrochlorinating agent and then chlorinating.

The drug is hydrolyzed to clofibric acid during absorption and in its pass through the liver, and it is the acid to which activity is attributed. The acid is bound strongly to plasma proteins. About 60% is metabolized, mostly to a glucuronide conjugate. The half-life is 6 to 25 hr (av 11 hr), except over 100 hr in anuria. Patients having the slower rates of metabolism have better clinical responses.

Suitable doses for use in adults are contemplated to be similar to those doses used to achieve an antihyperlipidemic effect, namely 500 mg 3 times a day for persons weighing less than 120 lb, 4 times a day for those weighing 120 to 180 lb, and 5 times a day for those over 180 lb, or to achieve an antidiuretic effect, namely 6 to 8 g/day in 2 to 4 divided doses. Clofibrate is available in 500 mg capsules.

2. Ciprofibrate

Several chemical relatives of clofibrate, collectively referred to as fibric acids, have proven to be less toxic and more effective for the treatment of hypertriglyceridemia and hypercholesterolemia than has clofibrate itself. One of these drugs is ciprofibrate.

3. Gemfibrozil

Gemfibrozil has been used extensively in the United States and Europe since the mid 1970s and was approved for use in the United States in 1982. Administration of a single dose of gemfibrozil (600 mg) results in a plasma concentration of about 15 Ug/ml after 2 hours and 5 Ug/ml after 9 hours. Final excretion occurs primarily through the kidneys, mainly as the glucuronide. Gemfibrozil (LOPID) is available as 300-mg capsules and 600-mg tablets. The usual recommended dosage (for adults only) is 600 mg twice daily, taken 30 minutes before the morning and evening meals.

4. Fenofibrate

A related compound, fenofibrate, is widely prescribed in Europe. The usual dosage is 100 mg orally after each meal. Administration of the drug with meals reduces the gastric irritation that occurs in a few patients.

5. Bezaflbrate

The structural formulas of clofibrate and the related fibric acid derivatives are shown in Table 1. Gemfibrozil, fenofibrate, bezafibrate, and ciprofibrate all are more potent than clofibrate and can be used in lower doses.

TABLE 1
Structural Formulas of Fibric Acids

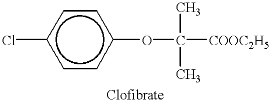

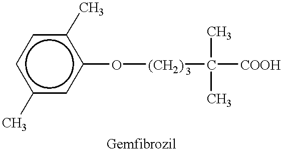

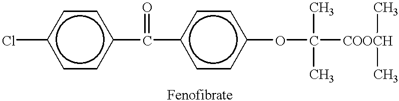

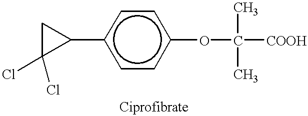

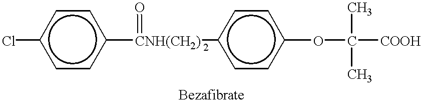

C. Anti-convulsants

1. Phenobarbital

Phenobarbital is 2,4,6(1H,3H,5H)-Pyrimidinetrione, 5-ethyl-5-phenyl-, Phenylethylmalonylurea, and is available from various commercial sources.

Phenobarbital is prepared by converting Benzyl chloride into phenylacetic ester (ethyl phenylacetate) by treating with sodium cyanide and then hydrolyzing with acid in the presence of alcohol. The ester is condensed in the presence of alcohol and metallic sodium with ethyl oxalate, forming diethyl sodium phenyloxaloacetate. HCl is added to liberate diethyl phenyloxaloacetate which, on being distilled at about 180° C., splits off carbon monoxide, and forms phenylmalonic ester [C₆H₅CH(COOC₂H₅)₂]. The hydrogen of the CH in the phenylmalonic ester is then ethylated and the resulting ethylphenylmalonic ester condensed with urea.

Approximately 80% of an oral dose is absorbed and peak plasma levels are reached in 16 to 18 hr. Therapeutic plasma levels range from 10 to 30 μg/mL. Apparent plasma half-life varies from 50 to 120 hr in adults. Suitable doses for use in adults are contemplated to include 30 to 120 mg in 2 or 3 divided doses; 100 to 320 mg and 50 to 100 mg 2 or 3 times a range of dose, 30 to 600 mg a day. Phenobarbital is available in many dosage forms: elixir; 15 or 20 mg/5 mL; capsules: 16 mg; tablets: 8, 16, 32, 65 and 100 mg.

Phenobarbital sodium is 2,4,6(1H,3H,5H)-Pyrimidinetrione, 5-ethyl-5-phenyl-, monosodium salt; and is available from Winthrop and various commercial sources. It can be prepared by dissolving phenobarbital in an alcohol solution of an equivalent quantity of NaOH and evaporating at low temperature. Because it is soluble in water, it may be administered parenterally. It may be given by slow intravenous injection. Suitable doses for adults include: intramuscular or intravenous 100 to 130 mg; 200 to 300 mg repeated in 6 hr if necessary. It is available in dosage forms of injection: 30, 60, 65 and 130 mg/mL; sterile powder, 120-mg ampuls.

2. Dilantin (Phenytoin)

Phenytoin is 2,4-Imidazolidinedione, 5,5-diphenyl-, Diphenylhydantoin and is available from Parke-Davis.

Suitable dose ranges are oral, 300 to 600 mg a day; usual, adult, 100 mg 3 times a day; the dose then is individualized. It is available in dosage forms of chewable tablets: 50 mg. Oral suspension: 30 mg/5 mL and 125 mg/5 mL.

Phenytoin Sodium is 2,4-Imidazolidinedione, 5,5-diphenyl-, monosodium salt also known as Alepsin; Epanutin; Eptoin and Dilantin Sodium, and is available from Parke-Davis. It is prepared by treating benzaldehyde with a solution of sodium cyanide, 2 moles of benzaldehyde are condensed (benzoin condensation) into one mole of benzoin, which is oxidized to benzil with nitric acid or cupric sulfate. The benzil is then heated with urea in the presence of sodium ethoxide or isopropoxide, forming phenytoin sodium.

Therapeutic plasma levels range from 10 to 20 μg/mL in adults and 5 to 20 μg/mL in children. Toxic levels range from 30 to 50 μg/mL and lethal levels approximate 100 μg/mL, these levels should be avoided.

There are two distinct forms of Phenytoin Sodium Capsules: the rapid-release type (Prompt Phenytoin Sodium Capsules) and the slow-dissolution type (Extended Phenytoin Sodium Capsules). The former have a dissolution rate of not less than 85% in 30 min and are used for 3 or 4 times a day dosing, whereas the latter have a slow dissolution rate of 15 to 35% in 30 min, 45 to 65% in 1 hr and not less than 85% in 2 hr and may be used for once-a-day dosing. Studies comparing doses of 100 mg three times a day of Prompt Phenytoin Sodium Capsules with a single, daily dose of 300 mg of Extended Phenytoin Sodium Capsules (Dilantin Kapseals, Parke Davis) indicate that absorption, peak plasma levels, biological half-life, difference between peak and minimum values and urinary recovery are equivalent.

Its metabolism may be altered significantly by concomitant use of other drugs. Drugs which increase its serum levels include chloramphenicol, dicumarol, tolbutamide, isoniazid, phenylbutazone, acute alcohol intake, salicylates, chlordiazepoxide, phenothiazines, diazepam, estrogens, ethosuximide, halothane, methylphenidate, sulfonamides, cimetidine and trazodone. Drugs which decrease its serum levels include carbamazepine, chronic alcohol abuse, reserpine and preparations containing calcium. Drugs which either increase or decrease its serum levels include phenobarbital, valproic acid and valproate sodium.

Suitable dose ranges are: oral, 200 to 600 mg a day; usual, oral, 100 mg up to 4 times a day; usual, intravenous, 150 to 250 mg, followed, if necessary, by 100 to 150 mg 30 min later (intravenous administration should not exceed 50 mg/min); usual, intramuscular, 100 to 200 mg every 6 to 8 hr for a total of 3 or 4 injections. Dosage forms—capsules: 100 mg (with phenobarbital 0.16 or 0.32 mg); prompt capsules: 30 and 100 mg; extended (once-a-day dosing) capsules: 30 and 100 mg.

3. Clonazepam

Clonazepam is 2H-1,4-Benzodiazepin-2-one, 5-(2-chlorophenyl)-1,3-dihydro-7-nitro-, and is available from Roche.

Clonazepam is prepared by reacting o-Chlorobenzoyl chloride with p-nitroaniline to form 2-amino-5-nitro-2′-chlorobenzophenone, and this is condensed with bromacetyl bromide to form 2-bromoacetamido-5-nitro-2′-chlorobenzophenone, then treated with ammonia to form the corresponding acetamido compound. The acetamido compound is converted to its hydrochloride with anhydrous HCl in methanol, dissolved in boiling methanol and cyclized to clonazepam using pyridine as the catalyst. Therapeutic plasma levels range from 20 to 80 ng/mL.

Suitable doses in adults are contemplated to include 1.5 mg in 3 divided doses; or 0.5 to 1.0 mg every 3 days. Maximum daily dose is 20 mg. It is available in dosage forms of tablets: 0.5, 1.0 and 2.0 mg.

4. Clotrimazole

Clotrimazole is 1H-Imidazole, 1-[2-chlorophenyl) diphenylmethyl]-also termed Lotrimin, available from Schering; and Mycelex, available from Miles. It is prepared from the reaction between imidazole and 2-chlorotriphenylmethyl chloride using trimethylamine as a proton receptor.

Suitable doses include, for adults, 5 g of 1% of cream or one 100-mg tablet daily, preferably at bedtime, for 7 to 14 consecutive days; 10 mg as a troche, slowly dissolved in the mouth 5 times a day. It is available in dosage forms of cream: 1%; vaginal cream: 1% (one applicator full contains 5 g of cream); topical solution: 1%; vaginal tablets: 100 and 500 mg; troches: 10 mg.

D. Corticosteroids

1. Dexamethasone

Dexamethasone is pregna-1,4-diene-3,20-dione, 9-fluoro-11,17,21-trihydroxy-16-methyl-, (11β,16α)-, and is available from various commercial sources. Miller et al. (1991) showed that oral dexamethasone can be used to advantage with verapamil in cancer treatment.

Suitable doses for use in adults include, initially, 500 μg to 9 mg a day in single or divided doses, and usually less; or 8 mg every other day for 1 mo; or 2 mg 2 or 3 times a day after parenteral dexamethasone sodium phosphate. It is available in dosage forms of topical aerosol: 0.01 and 0.04%; elixir: 0.5 mg/5 ML; gel: 0.1%; ophthalmic suspension: 0.1%; tablets: 0.25, 0.5, 0.75, 1, 1.5, 2, 4 and 6 mg.

Dexamethasone sodium phosphate is pregn-4-ene-3,20-dione, 9-fluoro-11,17-dihydroxy-16-methyl-21-(phosphonooxy)-,disodium salt, (11β,16α)-, dexamethasone 21- (Disodium Phosphate); also known as Decadron available from MSD; and Dalalone available from Forest.

It is one of the most soluble adrenocortical compounds. Thus, it lends itself well to intravenous administration, local injection and inhalation, and even to solutions and water-based ointments for topical application. Suitable doses are intravenous or intramuscular, adult, 420 μg to 7.5 mg a day, the dosage being decreased when a response occurs. Intra-articular, intralesional or soft-tissue injection, 170 μg to 5 mg. It is available in dosage forms of injection: 3.3, 8.33, 16.66; with various aerosols, sprays, creams, ointments and ophthalmic solutions also being available.

E. Oral Contraceptives

A variety of oral contraceptives may be employed, including Ethynodiol Diacetate; Levonorgestrel, available from Wyeth; Medroxyprogesterone Acetate, available from Upjohn or Reid Rowell; Norethindrone, available from Ortho, Syntex or Parke-Davis; Norethindrone Acetate, available from Ayerst or Parke-Davis; Norethynodrel, available from Searle; Norgestrel, available from Wyeth.

They are available in doses of Ethynodiol Diacetate/Ethinyl Estradiol tablets of 1 mg/35 or 50 μg; Ethynodiol Diacetate/Mestranol tablets of 0.5 or 1 mg/100 μg; Levonorgestrol/Ethinyl Estradiol, Monophasic tablets of 0.15/30 mg/μg; Triphasic tablets of 0.05/30, 0.075/40 and 0.125/30 mg/μg; Norethindrone/Ethinyl Estradiol, Monophasic tablets of 1 mg/50 μg, 1 mg/35 μg, 0.5 mg/35 μg, 0.4 mg/35 μg; Biphasic tablets of 0.5B mg/35 μg (×10) and 1 mg/35 μg (×11); Triphasic tablets of 0.5 mg/35 μg (×7), 1 mg/35 μg (×7) and 0.5 mg/35 μg (×7) or 0.5 mg/35 μg (×7), 0.75 mg/35 μg (×7) and d1 mg/35 μg (×7); Norethindrone/Mestranol, Monophasic tablets of 1/20, 1/50, 1/80, and 0.5 or 2/100 mg/μg. Biphasic tablets of 0/20 (×17) and 0.25/20 (×7) or 0/40 (×17) and 0.5/40 (×7) or 0/80 (×17) and 1/80 (×7) mg/μg; Norethindrone Acetate/Ethinyl Estradiol tablets of 1/20, 1.5/30, 1/50 and 2/50 mg/μg; Norethynodre/Mestranol tablets of 2.5:100, 5:75 and 9.85:150 mg:μg; and Norgestrel/Mestranol tablets of 500:50 and 300:30 μg:μg.

F. Retinoic Acids

It is retinoic acid, or so-called vitamin A acid, which is formed when the aldehyde group of retinene (retinal) is oxidized to a carboxyl group. Together with certain carotenoids, vitamin A appears to enhance the function of the immune system, to reduce the consequences of some infectious diseases, and to protect against the development of certain malignancies. As a result, there is considerable interest in the pharmacological use of retinoids for the prophylaxis of cancer and for the treatment of various premalignant conditions.

There are many types of preparations that contain retinol. Vitamin A capsules contain 3 to 15 mg of retinol (10,000 to 50,000 I.U.) per capsule; oral drops are also available. A water-miscible preparation (15 mg/ml; 50,000 I.U./ml) can be given intramuscularly. Isotretinoin (13-cis-retinoic acid; ACCUTANE) is available for oral use as 10-, 20-, and 40-mg capsules. The initial daily dose is usually 0.5 to 1 mg/kg in two divided doses up to a maximum of 2 mg/kg. A course of therapy is usually 15 to 20 weeks; which may be repeated after an interval of 2 months. Etretinate (TEGISON) is available for oral use as 10- and 25-mg capsules. Initial daily doses are usually 0.75 to 1 mg/kg up to a maximum of 1.5 mg/kg.

G. Rifampin

Rifampin includes Rifamycin, 3-[[(4-methyl-1-piperazinyl)imino]methyl]-, Rifampicin; Rifadin available from Merrell Dow; and Rimactane available from Ciba-Geigy.

Rifamycin SV, which may be prepared by the method of Sensi et al. (U.S. Pat. No. 3,313,804), is converted to the 8-carboxaldehyde derivative, known also as 3-formylrifamycin SV, and this is condensed with 1-amino-4-methylpiperazine in a Schiff base reaction to yield rifampin.

Metabolism is dose-dependent with doses above 300 to 450 mg; with therapeutic doses the serum half-life is 1.5 to 5 hr. Even so, the drug is usually administered at 8- to 12-hr intervals, because absorption is slow enough to sustain effective levels for 8 to 10 hr.

Suitable adult doses include, orally, 600 mg once a day, taken with a glass of water at least 1 hr before a meal; elderly or debilitated patients, 10 mg/kg once a day. It is available in dosage forms of capsules: 150 and 300 mg; capsules (in combination with isoniazid): 300 mg rifampin and 150 mg isoniazid; tablets: 300 mg.

H. Disulfiram (Antabuse)

Disulfiram is Thioperoxydicarbonic diamide, tetraethyl-, Tetraethylthiuram Disulfide, known as Antabuse, available from Ayerst. Disulfiram is prepared by treating a cold solution of diethylamine and carbon disulfide in alcohol with an alcoholic solution of iodine. Ice water way be added to hasten separation of the disulfiram. Suitable doses are usually, oral, initially up to 500 mg a day for the first 2 or 3 wk; usual, maintenance, 250 mg a day. It is available in dosage forms of tablets: 250 and 500 mg.

Biliary Transport Protein Inhibitors

A. Cyclosporines And Staurosporines

1. Cyclosporines

Cyclosporine A is known to be an inhibitor of biliary excretion mediated by both cMOAT and p-glycoprotein (Liu, et al. 1996).

Pourtier-Manzanedo et al. (1992) reported that the non-immunosuppressive cyclosporine derivative, SDZ PSC 833 (valspodar), inhibited p-glycoprotein. These authors also showed that the semi-synthetic cyclopeptolide, SDZ 280-446, was a p-glycoprotein blocker.

Boesch et al. (1991) also showed that SDZ PSC 833 (valspodar) was an effective inhibitor. In studies with target cells, PSC 833 (valspodar) was at least one order of magnitude more active than cyclosporine A in restoring drug sensitivity of multi-drug resistance (MDR) P388 cells.

Boesch & Loor (1994) later reported that SDZ PSC 833 (valspodar), SDZ 280-446, cyclosporine A and verapamil were effective at inhibiting p-glycoprotein. CsA, SDZ 280-446 and SDZ PSC 833 (valspodar) were shown to be stronger inhibitors than verapamil, with SDZ PSC 833 (valspodar) still exhibiting inhibition of p-glycoprotein function even two days after a single pulse exposure. The studies of Boesch & Loor (1994) regarding the persistence of Pgp inhibition conferred by some agents can thus be combined with the teachings herein to allow even more effective clinic protocols to be designed.

Cyclosporin A [59865-13-3] C₆₂H₁₁₁N₁₁O₁₂ (1202.63) is used to suppress helper T-lymphocytes without significantly affecting suppressor T-lymphocytes or B-lymphocytes. Thus, it is a selective immunosuppressive drug without the cytotoxicity characteristic of most other immunosuppressive drugs. It has a modest effect to suppress some humoral immunity.

It is the most efficacious immunosuppressive for prevention of graft rejection in allogenic transplantation of kidney, liver or heart. It is used also in the management of severe aplastic anemia, some cases of myasthenia gravis, childhood diabetes (Type I) of recent onset, Graves' disease, Crohn's disease, multiple sclerosis, pemphigus and pemphigoid, dermatomyositis, polymyositis, atopic dermatitis, severe psoriasis, Bechcet's disease, uveitis, biliary cirrhosis and pulmonary sarcoidosis. It usually is employed in combination with a glucocorticoid. Although combination with other immunosuppressives usually is avoided, in bone-marrow transplantation it commonly is combined with methotrexate.

Doses are, intravenous infusion, adults and children, 2 to 6 mg/kg/day, starting 4 to 12 hr before transplantation and continuing until oral dosage can be tolerated. Oral, adults and children, initially 12 to 15 mg/kg/day starting 4 to 12 hr before transplantation or after IV infusion and continuing for 1 to 2 wk, after which the dose is diminished by 5% wk to a maintenance dose of 5 to 10 mg/kg/day. It is available in injectable dosage forms of 250 mg/5 mL; and oral solutions of 5 g/100 mL.

3′-Keto-cyclosporine D, which has negligible immunosuppressive activity, also strongly inhibits p-glycoprotein of multi-drug resistant mammalian tumor cells (Bell et al., 1994) and thus may be in the present invention.

2. Cefoperazone

Cefoperazone is a third-generation cephalosporin with antibacterial activities. It is approved for use in urinary tract infections caused by Enterobacter, P. aeruginosa and anaerobic cocci and bacilli; and in respiratory tract infections caused by Enterobacter, E. coli and other organisms.

Orally, it is absorbed poorly. An intravenous dose yields a peak plasma concentration of 250 to 357 μg/mL depending on the rate of delivery, an intramuscular dose 80 to 120 μg/mL. In plasma, 82 to 93% is protein-bound. The volume of distribution 0.13 to 0.20 mL/g in adults but 0.5 mL/g in neonates. Biliary secretion eliminates 70% and urinary excretion 30% of the drug. Dose adjustments are needed in hepatic but not in renal failure.

Doses of Cefoperazone Sodium A (in cefoperazone equivalents are: intramuscular or intravenous infusion, adults, for mild infections, 1 to 2 g every 12 hr and, for severe infections, 1.5 to 3 g every 6 hr, 2 to 4 g every 8 hr or 3 to 6 g every 12 hr. It is available in powder forms for injection of 1 and 2 g.

3. Staurosporins

Staurosporine derivatives, such as NA-382, also inhibit multidrug resistance by inhibiting p-glycoprotein (Miyamoto et al., 1993). The effects of the staurosporine derivative, N-ethoxycarbonyl-7-oxo-staurosporine (NA-382) on multidrug resistance in tumor cells were shown to be due to inhibiting drug binding to p-glycoprotein. Therefore, both staurosporine and NA-382 may also be used as second agents in accordance with flavopiridols.

B. Calcium Channel Blockers

Calcium channel blockers, also termed calcium entry blocking drugs (CEBs) or calcium antagonists, are a group of agents whose main pharmacological effect is to prevent or slow the entry of calcium into cells via specialized calcium channels. Five of these drugs are available in the U.S.: verapamil, nifedipine, nitrendipine, nicardipine and diltiazem. Nifedipine and nitrendipine are dihydropyridines, a chemical class to which most new calcium channel blockers belong.

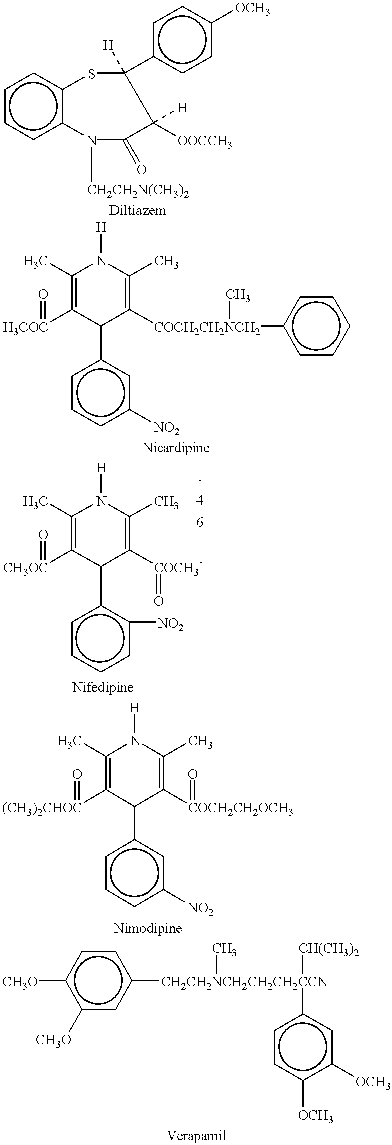

1. Dihydropyridine Analogues

Certain dihydropyridine analogues inhibit p-glycoprotein, as shown by inhibiting photolabeling of p-glycoprotein in human cells (Kamiwatari et al., 1989). Dihydropyridine analogues have also been reported to overcome MDR in cells overexpressing MDR-associated protein (MRP) mRNA (Tasaki, 1995) The seven best inhibitory dihydropyridines described by Kamiwatari et al. (1989) are contemplated for use in the invention. The cationic compounds cepharanthine and reserpine also showed inhibition in this study.

2. Verapamil

Verapamil is known to be a competitive inhibitor of p-glycoprotein and MRP, as described by Ling (1997), Inoue et al. (1993); Hunter et al. (1993); Hori et al. (1993); Pourtier-Manzanedo et al. (1992); Boesch & Loor (1994); Zacherl et al (1994); Shirai et al. (1991); Morris et al. (1991); Muller et al. (1994); and Miyamoto et al. (1992b).

Thalhammer et al. (1994) showed that p-glycoprotein-mediated transport of the cationic dye, acridine orange, across the bile canaliculi was inhibited by cyclosporine A and verapamil. The ATP-dependent transport of amphiphilic cations across the hepatocyte canalicular membrane by p-glycoprotein was also studied by Muller et al. (1994). Transport of permanently charged amphiphilic cations was inhibited by verapamil, quinidine and daunorubicin.

Bear (1994) showed that verapamil, colchicine, vinblastine daunomycin and (50 microM) blocked an outwardly-rectifying chloride channel that was proposed to be associated with p-glycoprotein expression.

Ohi et al. (1992) used the calcium-channel blocker, verapamil, with adriamycin in chemotherapy for superficial bladder cancer. Five ampules (10 ml) of injectable verapamil were given.

Verapamil hydrochloride is benzeneacetonitrile, α-[3-[[2-(3,4-dimethoxyphenyl)ethyl]-methylamino]propyl]-3,4-dimethoxy-α-( 1-methylethyl)-, hydrochloride; also termed Calan and Isoptin, and available from Searle, Knoll and Parke-Davis.

It is more than 90% absorbed, but only 20 to 35% of the dose reaches the system because of extensive hepatic first-pass metabolism. It is bound approximately 90% to plasma proteins. It is metabolized rapidly by the liver to norverapamil and traces of several other metabolites. About 70% of a dose is excreted in urine as metabolites, and 16% of a dose appears in the feces within 5 days; less than 5% is excreted unchanged.

The effects of verapamil are evident within 30 to 60 minutes of an oral dose. Peak effects of verapamil occur within 15 minutes of its intravenous administration. The half-life is 1.5 to 5 hours in normal persons but may exceed 9 hr during chronic therapy. In patients with cirrhosis of the liver, the half-life may be increased to 14 to 16 hr. The half-life is increased in patients with liver disease, due, in part, to an increased volume of distribution. Saturation kinetics have been observed after repeated doses.

Doses are: intravenous, adults, initially 5 to 10 mg (0.075 to 0.15 mg/kg) over a period of 2 min (3 min in the elderly), followed by 10 mg (0.150 mg/kg) after 30 min, if necessary; children, up to 1 yr, initially 0.1 to 0.2 mg/kg over 2 min (with ECG monitoring), repeated after 30 min, if necessary; 1 to 15 yr, initially 0.1 to 0.3 mg/kg, not to exceed 5 mg, repeated after 30 min, if necessary. Oral, adults, 80 mg 3 or 4 times a day or 240 mg once a day in sustained-released form, gradually increased to as much as 480 mg a day, if necessary.

Verapamil is available in injectable dosage forms of 5 mg/2 mL and 10 mg/4 mL; tablet dosage forms of 40 mg, 80 mg and 120 mg; and sustained-telease tablets of 240 mg.

3. Tiapamil

Campain et al. (1993) reported that the tiapamil analog, RO-11-2933, is an inhibitor of p-glycoprotein. A tiapamil analogue was also described as an efflux-blocking drug by Williams et al. (1992). Tiapamil is proposed for use at doses generally equivalent to those of verapamil.

4. Nifedipine

Wilson et al. (1991) reported that nifedipine is a p-glycoprotein inhibitor that is structurally unrelated to verapamil. However, both nifedipine and verapamil belong to the group of calcium channel blockers. Hunter et al. (1993) and Morris et al. (1991) also showed nifedipine to be a p-glycoprotein inhibitor.

Nifedipine is 3,5-Pyridinecarboxylic acid, 1,4-dihydro-2,6-dimethyl-4-(2-nitrophenyl)-, dimethyl ester; also termed Adalat and Procardia, available from Miles and Pfizer, respectively. About 90% of an oral dose is absorbed, but its bioavailability is 65 to 70%; there is significant hepatic first-pass metabolism. Greater than 90% of the drug is bound to plasma protein. It is metabolized to inactive metabolites, probably by the liver. Most (80%) of the inactive metabolites are excreted in urine; 15% are excreted in the stool. The half-life is 2 to 6 hours.

Oral doses are, for adults initially 10 mg 3 times a day, to be increased gradually to 20 to 30 mg three or four times a day, if necessary. The usual effective dosage is 10 to 20 mg three times daily, but 20 to 30 mg taken three or four times daily may be necessary. Doses exceeding 180 mg a day are not recommended. It is available in capsule dosage forms of 10 and 20 mg.

5. Diltiazem

Morris et al. (1991) identified diltiazem as a p-glycoprotein inhibitor, along with verapamil, nifedipine and vinblastine.

Diltiazem is benzothiazepin-4(5H)-one, 3-(acetyloxy)-5-[2-(dimethylamino)ethyl]-2,3-dihydro-2-(4-methoxyphenyl)-, (+)-cis-, monohydrochloride, also termed Cardizem available from Marion. It is 80% absorbed orally, but only 40 to 60% of an oral dose reaches the systemic circulation because of first-pass metabolism in the liver. After administration, it is 70 to 80% bound to plasma protein. Displacement from protein binding sites by other drugs does not seem to be a clinical problem. It is metabolized extensively by the liver to several metabolites, some of which have weak coronary vasodilator activity. Less than 4% of the drug appears unchanged in the urine. The plasma half-life is about 4 hr.

Oral doses are, for adults, initially: 30 mg 4 times a day before meals and at bedtime, to be increased to 360 mg day, as necessary. The sustained-release preparation is given twice daily. It is available in tablet dosage forms of 30, 60, 90 and 120 mg.

6. Nicardipine

Nicardipine is also proposed for use in the present invention, based on its similarity with the above calcium channel blockers and in light of the studies by Niwa et al. (1992).

Nicardipine has pharmacodynamic and pharmacokinetic properties close to those of nifedipine, and is proposed for use at similar doses. Nicardipine is an effective antianginal and antihypertensive agent.

Nicardipine hydrochloride (CARDENE) is available in 20- and 30-mg tablets for use in hypertension and angina. The recommended dosage is 20 to 40 mg three times a day. At least 3 days should elapse between adjustments of dosage.

7. Nisoldipine

Nisoldipine is also similar to Nicardipine, with properties close to nifedipine, and having effective antianginal and antihypertensive uses. Nisoldipine is proposed for use with flavopiridols in the present invention, in a similar manner to nicardipine and nifedipine.

8. Nimodipine

Nimodipine is also proposed for use in the present invention, based on its similarity with other calcium channel blockers. Nimodipine has effects on cerebral blood vessels. It selectively dilates cerebral vessels but has only minor effects on peripheral circulation. It is useful in the treatment of cerebral arterial spasm following subarachnoid hemorrhage, migraine headache, acute ischemic stroke and severe head injury. It is also proposed for use in the present invention, similarly to nisoldipine, nicardipine and nifedipine.

Nimodipine (NIMOTOP) is available in 30-mg capsules. The approved indication for its use is to improve neurological deficits due to vasospasm following subarachnoid hemorrhage from ruptured congenital intracranial aneurysms. The recommended dosage is 60 mg every 4 hours for 21 days, beginning within 96 hours of the hemorrhage.

9. Nitrendipine

Nitrendipine is 3,5-Pyridinedicarboxylic acid, 1,4-dihydro-2,6-dimethyl-4-(3-nitrophenyl)-, ethyl methyl ester; available as Baypress from Miles.

Nitrendipine is approved for the treatment of mild to severe hypertension. It is metabolized extensively in the liver, and only 10 to 22% of an oral dose reaches the systemic circulation in young adults. The elimination half-life has been reported to range from 2 to 23 hr, 12 hr probably being an average value. Hepatic dysfunction greatly increases the half-life. The volume of distribution has been reported variously to be 2 to 6 L/kg. About 98% is bound to plasma proteins.

It is used in dosage forms of, for adults, initially 5 to 20 mg once a day in the morning, to be adjusted to twice this dose, if necessary. Nitrendipine is also proposed for use in the invention, by virtue of its similarity to the above calcium channel blockers.

C. Calmodulin Antagonists

Ford et al. (1990) indicated that thioxanthenes and phenothiazines have uses in inhibiting p-glycoprotein.

1. Flupenthixols

Phenothiazines have been shown to sensitize multidrug resistant (MDR) cells to chemotherapeutic drugs in a manner related to specific structural features. Ford et al. (1990) identified structurally related phenothiazines and thioxanthenes with increased anti-MDR activity. Any of the compounds in the Ford study are proposed for use in the present invention.

Ford et al. (1990) particularly showed that trans-flupenthixol, its stereoisomer cis-flupenthixol, its phenothiazine homologue fluphenazine, and the calcium channel blocker verapamil, reversed cellular resistance to various drugs only in p-glycoprotein expressing cell lines. trans-flupenthixol caused a greater reversal of cellular resistance to doxorubicin, vinblastine, vincristine, and colchicine, and was two- to three-fold more potent for reversing MDR than equimolar concentrations of verapamil. Furthermore, trans-flupenthixol fully reversed resistance to doxorubicin, vincristine, and colchicine.

The apparent lack of clinical toxicity of trans-flupenthixol makes it an attractive drug for possible use in the modulation of tumor resistance in vivo (Hait et al., 1993). The present inventors thus propose that trans-flupenthixol would be particularly effective for combination with flavopiridols, as disclosed herein. Cis-flupenthixol and clorpenthixol are also contemplated for use.

2. Fluphenazine

In light of the studies of Ford et al (1990), fluphenazine is proposed for use in the present invention. Fluphenazine hydrochloride, also termed Permitil and Prolixin, is available from Schering-Plough and Squibb. It is a trifluoromethyl phenothiazine derivative intended for the management of manifestations of psychotic disorders. Although the pharmacological effects are, in general, similar to those of other phenothiazines, laboratory and clinical studies indicate that this drug exhibits several important differences. The drug is more potent, exhibits a more prolonged duration of action, is less likely to induce hypotension, is less sedative and does not potentiate CNS depressants and anesthetics to the same degree as other phenothiazines.

It is absorbed rapidly after oral or intramuscular administration, onset of action occurs within 1 hr, peak plasma levels in 1.5 to 2 hr and duration of action is 6 hr. The intramuscular or subcutaneous administration of the enanthate salt has an average duration of 2 wk.

It is used in oral doses of, adult, initially 0.5 to 10 mg a day in divided doses; maintenance, 1 to 5 mg as a single dose a day; intramuscular, 1.25 to 10 mg a day divided into 4 doses. Daily dosages exceeding 20 mg orally or 10 mg intramuscularly should be used with caution. It is available in elixir dosage forms of 1 mg/2 mL; concentrated, 5 mg/mL; injectable, 25 mg/10 mL; tablets, 1, 2.5, 5 and 10 mg.

Fluphenazine decanoate, also termed prolixin decanoate, is available from Princeton. It is a trifluoromethyl phenothiazine derivative indicated for the management of patients requiring prolonged parenteral neuroleptic therapy (eg, chronic schizophrenics). Peak plasma level usually is achieved in 1 to 2 days; half-life (after a single dose) is 6.8 to 9.6 days, onset of action is 1 to 3 days and duration of action is about 4 wk.

Useful doses are, intramuscular or subcutaneous, 12.5 to 100 mg; usually, 12.5 to 25 mg; subsequent injections and dosage interval are based with patient response. It is available in injectable dosage forms of 25 mg/mL vials and 1-mL Unimatic syringes.

Fluphenazine enanthate (prolixin enanthate) except for duration of action, it has actions, uses, contraindications, and untoward effects similar to those of the hydrochloride. The esterification of fluphenazine with the enanthate moiety markedly prolongs the drug's duration of action without unduly attenuating its beneficial effects. The onset of action generally appears between 24 to 72 hr after injection and the effects of the drug on psychotic symptoms become significant within 48 to 96 hr. Amelioration of symptoms continues for 1 to 3 wk or longer, with an average duration of effect of about 2 wk.

Suitable doses are, intramuscular or subcutaneous, 12.5 to 100 mg every 1 to 3 wk; usual, 12.5 to 25 mg; subsequent injections and dosage interval are based on patient response. It is available in injectable dosage forms 25 mg/mL in 5-mL vials.

3. Chlorpromazine

Akiyama et al. (1988) showed that chlorpromazine and trifluoperazine effectively reverse multidrug resistance, most probably by binding to p-glycoprotein. However, these agents were poor inhibitors of the photoaffinity labeling of p-glycoprotein. This suggests that although most agents that phenotypically suppress multidrug resistance also inhibit photoaffinity labeling of p-glycoprotein, some may not have this property. Binding and inhibition of p-glycoprotein may thus occur at distinct sites on the molecule. This is also supported by monoclonal antibody data.

Chlorpromazine hydrochloride is 10H-Phenothiazine-10-propanamine, 2-chloro-N,N-dimethyl-, mono hydrochloride. It was the first tranquilizer of the phenothiazine group of compounds and is effective in the management of manifestations of psychotic disorders, nausea and vomiting, manifestations of manic depressive illness (manic phase), intractable hiccups, apprehension and anxiety prior to surgery, acute intermittent porphyria and as an adjunct in the treatment of tetanus. The volume of distribution has been reported to be 21.8 L/kg after intramuscular administration and 80.6 L/kg after a single oral dose. This 4-fold difference reflects the low bioavailability via the route (32%).

Dosage is variable and requires strict individualization. Administration is oral, intramuscular, or intravenous. Parenteral administration should be reserved for bedfast or hospitalized patients. If used in ambulatory patients, the patient must remain in a supine position for at least 1 hr after the injection.

Appropriate doses are, as an antiemetic, adults, oral, 10 to 25 mg every 4 to 6 hr.; intramuscular, 25 to 50 mg every 3 ro 4 hr until vomiting ceases. Children, oral, 0.5 mg/kg every 4 to 6 hr; intramuscular, 0.5 mg/kg every 6 to 8 hr as required. Tranquilizer, adults, oral usual, 10 to 50 mg 2 or 3 times a day to a total dose of 1 g a day when indicated: intramuscular, 25 to 50 mg, repeated in 1 hr if necessary to a total dose of 1 g a day when indicated. Children, oral, 0.5 mg/kg every 4 to 6 hr; intramuscular, 0.5 mg/kg every 6 to 8 hr as required.

It is available in injectable dosage forms of 25 mg/mL in 1,2 and 10 mL; timed-release capsules of 30, 75, 150, 200 and 300 mg; syrup of 2 mg/mL; concentrate of 30 and 100 mg/mL; and tablets of 10, 25, 50, 100 and 200 mg.

Chlorpromazine, also termed thorazine, is available from SmithKline. Doses are, as an antiemetic used rectally, 50 to 100 mg every 6 to 8 h. Dose range, 50 to 400 mg. Pediatric, antiemetic, children 6 mo and older, 1 mg per kg or ½ of a 25 mg suppository 3 or 4 times a day as necessary; children under 6 mo not recommended. It is available in suppository dosage forms of 25 and 100 mg. Clomipramine may also be used in a similar manner. 4. Triflupromazine

Triflupromazine hydrochloride is 1OH-Phenothiazine-10-propanamine, N,N-dimethyl-2-(trifluoromethyl)-monohydrochloride; also termed versprin hydrochloride and available from Squibb. It is the 2-(tribluoromethyl) analogue of chlorpromazine hydrochloride and is indicated for the management of psychotic disorders (excluding psychotic depressive reactions) and for the control of severe nausea and vomiting.

Except that this drug is somewhat more potent, it has the same actions and limitations as chlorpromazine. In light of the studies of Akiyama et al. (1988), triflupromazine is also proposed for use in the p-glycoprotein inhibition aspects of the present invention.

Suitable doses are, for psychotic disorders, usual, adult, intramuscular, initially 60 to 150 mg in divided doses; intravenous, 1 mg, up to a 3-mg total daily dose. Children, intramuscular, 0.2 to 2.5 mg/kg a day: not to exceed 10 mg a day. Nausea and vomiting, usual, adult, intramuscular, 5 to 15 mg as a single dose; may be repeated every 4 hr but not to exceed 60 mg/day. Elderly or debilitated patients, intramuscular, 2.5 mg, not to exceed 15 mg/day. Children, 2½ yr and older, intramuscular, 0.2 to 0.25 mg/kg a day in divided doses, not to exceed 10 mg a day. It is available in injectable dosage forms of 10- and 20-mg/mL in multiple-dose vials; and in tablets of 10, 25 and 50 mg.

5. Trifluoperazine

Trifluoperazine reverses multidrug resistance without inhibiting photoaffinity labeling of p-glycoprotein (Akiyama et al., 1988).

Trifluoperazine is 10H-Phenothiazine, 10-[3-(4-methyl-1-piperazinyl)propyl]-2-(trifluoromethyl)-, dihydrochloride; also termed stelazine and suprazine. It is a piperazine phenothiazine effective in the management of the manifestations of psychotic disorders. It is possibly effective for the control of excessive anxiety, tension and agitation seen in neurosis or associated with somatic conditions. The general profile of pharmacological action is similar to other phenothiazine derivatives. Bioavailability, time to peak effect, metabolism and elimination half-life resemble those for chlorpromazine. Untoward effects such as hypotension, blurred vision and other manifestations of autonomic blockade appear to be less troublesome than with other phenothiazines.

Suitable doses are: oral, non-hospitalized patients, 1 to 2 mg twice a day; hospitalized patients, 2 to 5 mg twice a day initially, gradually increasing to the optimum level of 15 to 20 mg a day, although a few patients may require 40 mg a day or more; intramuscular, 1 to 2 mg every 4 to 6 hr as required. Elderly patients, lower doses are usually sufficient; the elderly are more susceptible to hypotension and neuromuscular reactions, observe closely and increase dosage gradually. Nonpsychotic anxiety, 1 to 2 mg twice a day; maximum, 6 mg/day, not to exceed 12 wk. (Doses stated in base equivalents.) Usual, pediatric, oral, hospitalized children 6 to 12 yr, 1 mg once or twice a day, dosage gradually increased until symptoms controlled; maximum, 15 mg a day. It is available in injectable dosage forms of (base equivalent) 20 mg/10 mL; concentrate, 10 mg/mL; tablets, 1, 2, 5 and 10 mg.

6. Prochlorperazine

Prochlorperazine maleate is used as an antiemetic, antipsychotic and tranquilizing agent. It is an effective antiemetic in the control of mild or severe nausea and vomiting due to a variety of causes, such as early pregnancy, anesthesia and surgery and radiation therapy. The drug is also an effective antipsychotic and is used in severe psychiatric disorders such as schizophrenia, mania, involutional psychoses, degenerative conditions and senile and toxic psychoses. As a tranquilizing agent, it is possibly effective in mild mental disorders in which anxiety, tension and agitation predominate.

In light of the studies of Ford et al. (1990) and Akiyama et al. (1988), prochlorperazine is envisioned for use with flavopiridols in the present invention.

Appropriate doses are (as base equivalent), in adults, oral, antiemetic, 5 to 10 mg 3 or 4 times a day as required; tranquilizer, 5 to 35 mg 3 or 4 times a day, the initial low dose being increased gradually until the desired response is obtained, for which 50 to 150 mg daily usually is required. It is available in tablet dosage forms of (base equivalent): 5, 10 and 25 mg; Sustained Release Capsules: 10, 15, 30 and 75 mg from Major and Smith Kline & French.

Prochlorperazine, as the base, is also administered rectally at doses of, for children, 2.5 to 10 mg a day, according to weight, in divided doses; adults, 25 mg 2 times a day. The Child's rectal dose should not exceed 7.5, 10 and 15 mg a day for a 20- to 29-lb, 30- to 39-lb and 40- to 58-lb children, respectively. It is not recommended for children weighing less than 20 lb. It is available in suppository dosage forms of 2.5, 5, 10 and 25 mg.

Prochlorperazine edisylate is also available for use as prochlorperazine maleate, except that it may be administered intramuscularly as well as orally. Parenteral therapy usually is reserved for the treatment of severe nausea and vomiting, for the immediate control of acutely disturbed psychotics or for patients who cannot or will not take oral medication.

It is given in oral dosage forms of (as base equivalent), antiemetic, 5 to 10 mg 3 or 4 times a day as required; tranquilizer, 5 to 35 mg 3 or 4 times a day. Usual range of oral dose, 5 to 150 mg daily. Intramuscular or intravenous, antiemetic, 5 to 10 mg 6 to 8 times a day as required; tranquilizer, 10 to 20 mg 4 to 6 times a day as required. Usual range of parenteral dose, as antiemetic, 5 to 40 mg daily; as tranquilizer, 10 to 200 mg daily. No more than 40 mg of base equivalent should be injected in any 24-hr period unless the patient is hospitalized and under adequate observation. For acutely disturbed patients, the usual dose is 20 to 40 mg intramuscularly at intervals of 1 to 6 hr. It is available in injectable dosage forms of (base equivalent) 5 mg/mL; syrup forms of 5 mg/5 mL; and concentrate (for institutional use): 10 mg/mL.

7. Thioridazine

Thioridazine is 10H-Phenothiazine, 10-[2-(1-methyl-2-piperidinyl)ethyl]-2-(methylthio)-, also termed Mellaril-S and available from Sandoz. It is a piperidyl-type phenothiazine tranquilizer with central sedative and behavioral effects similar to those of chlorpromazine. Half-life appears to be multiphasic with an early phase of 4 to 10 hr and a late phase of 26 to 36 hr; 96 to 99% is bound to plasma protein. As thioridazine has similar effects to chlorpromazine, it is considered to be suitable for use in binding to and inhibiting p-glycoprotein in accordance with the present invention.

Suitable doses are, adult, usual, initially 25 to 100 mg three times a day; maintenance, 10 to 200 mg 2 to 4 times a day. For the management of agitation, anxiety, depressed mood, tension, sleep disturbances and fears in geriatric patients, usual, oral 25 mg 3 times a day. Total daily dose ranges from 200 to 800 mg, divided into two to four doses. Usual, pediatric, children 2 to 12 yr, 0.5 to a maximum of 30 mg/kg/day, dosage increased daily until optimum therapeutic effect obtained or the maximum dose reached. It is available in concentrated dosage forms of 30 and 100 mg/mL; as a suspension of 25 and 100 mg/5 mL; or as tablets of 10, 15, 25, 50, 100, 150 and 200 mg.

D. Steroids

1. Progesterone and Metabolites

Ichikawa-Haraguchi et al. (1993) identified progesterone and its metabolites as potent inhibitors of the transporting activity of p-glycoprotein in the adrenal gland. These authors reported that progesterone and pregnenolone inhibited the transporting activity of p-glycoprotein. Six authentic progesterone metabolites in the 5 beta-metabolic pathway were also able to inhibit p-glycoprotein. Stereoisomerism around carbon 5 of the progesterone metabolites is important for them to be recognized by p-glycoprotein. Progesterone and pregnenolone analogues of the correct stereoisomerism are thus proposed for use in the present invention.

2. RU 486

Gruol et al. (1994) also reported that progesterone binds p-glycoproteins and inhibits their drug efflux. It is further reported that the antiprogestin, RU 486, reverses multidrug resistance in cells with p-glycoprotein and MRP (Payen, et al. 1999). In measuring the inhibition of p-glycoprotein-dependent drug efflux, RU 486 is found to be considerably more effective than progesterone and one-half as effective as verapamil (Gruol et al. 1994). RU 486 is thus also proposed for use herewith.

3. Tirilazad

Non-glucocorticoid steroid analogues (21-aminosteroids) also sensitize multidrug resistant cells to vinblastine (Abraham et al., 19932). These 21-aminosteroid derivatives, also termed lazaroids, include te potent inhibitors tirilazad mesylate (tirilazad, U-74006F) and U-74389F. Tirilazad sensitizes resistant cells to killing by vinblastine by 66-fold, but does not change the sensitivity of nonresistant parental cells. Tirilazad inhibits the photoaffinity labeling of p-glycoprotein, more effectively than does verapamil. Studies suggest that the complex amine portion of tirilazad is important for its reversal activity, while the steroid portion is less important. Therefore, tirilazad and other structurally related compounds, e.g., those developed to treat stroke and trauma of the central nervous system are envisioned to be useful in the present invention.

E. Cationic Compounds

1. Reserpine

In studying digoxin transport by p-glycoprotein, Hori et al. (1993) showed that reserpine inhibited this process. Akiyama et al. (1988) and Miyamoto et al (1992b) also showed that reserpine binds to p-glycoprotein and inhibits photo-labeling.

Reserpine is Yohimban-16-carboxylic acid, 11,17-dimethoxy-18-[(3,4,5-trimethoxybenzoyl)oxy]-, methyl ester, (3β, 16β, 17α, 18β, 20α)-. It is used as a tranquilizer and as an antihypertensive agent. Because the hypotensive doses used are generally considerably smaller than those for its tranquilizing effects, reserpine is used for its hypotensive effects with more safety than as a psychopharmacological drug. It exerts its antihypertensive effects through a partial depletion of the norepinephrine in the sympathetic postganglionic nerves. Intravenously, it is quite useful in the management of severe hypertension and hypertensive crises.

The drug is absorbed poorly and erratically from the gastrointestinal tract, which causes a considerable difference in efficacy of oral vs. intravenous doses. It characteristically has a long latency of onset and a prolonged duration of action. for example, with daily oral administration the effects of the drug usually are not fully manifest for several days to 2 weeks and may persist for as long as 4 weeks after oral medication is discontinued. Tolerance to the drug does not develop with continued administration.

Suitable doses are, oral, adults, for hypertension, initially 0.05 to 0.2 mg a day in 1 or 2 divided doses; when higher doses are used, the patient must be monitored continuously for mood depression, for anxiety-tension, initially 0.1 to 0.5 mg a day for control, then adjusted to minimum effective dose; for psychotic disorders, initially 0.1 to 1 mg a day, with subsequent adjustments to maintain control; children (not recommended), for hypertension, 5 to 20 μg/kg (or 150 to 600 μg/m²) a day, once or in 2 divided doses. It is available in extended-release capsules of 0.5 mg; and as tablets of 0.1, 0.25 and 1 mg.

2. Dipyridamole

Tatsuta et al (1991) showed that the activities of anti-tumor drugs against multidrug-resistant human hepatoma cells is enhanced by dipyridamole. Dipyridamole (DPM), at 10 μM enhanced the cytotoxicity of anti-tumor drugs, and increased dose-dependently the intracellular accumulation of vinblastine. It was concluded that DPM binds to p-glycoprotein and inhibits active drug efflux. Suzuki et al. (1990) also reported dipyridamole to be a p-glycoprotein inhibitor.

Dipyridamole is [2,6-Bis(diethanolamino)-4,9-dipiperidinopyrimido-(5,4-d)pyrimidine[58-32-2] C₂₄H₄₀N₈O₄, which is available from Boehringer Ingelheim. It is used to inhibit phosphodiesterase, the synthesis of thromboxane A₂, the reuptake of adenosine and promotes the synthesis of prostacyclin in vascular smooth muscle. In coronary blood vessels, all of these actions favor coronary vasodilation. The drug is approved for adjunctive use with prothrombopenic anticoagulants in the postoperative prevention of thromboembolism in cardiac valve replacement. It also is used with aspirin or warfarin in coronary bypass surgery and with aspirin in transient ischemic attacks, after myocardial infarction and in deep vein thrombosis.

Suitable doses are, oral, adult, 75 to 100 mg 4 times a day. It is available in tablet dosage forms of 25, 50 and 75 mg. In the doses usually employed clinically, dipyridamole is quite nontoxic.

3. Chloroquine

Chloroquine partially reverses drug resistance in multidrug-resistant human carcinoma cells, and for this reason is contemplated for use in this invention (Akiyama et al., 1988). Chloroquine phosphate is 1,4-Pentanediamine, N⁴-(7-chloro-4-quinolinyl)-N¹, N¹-diethyl-, phosphate (1:2); available from Biocraft, Danbury, and also termed Aralen Phosphate, available from Winthrop.

It is used as an antimalarial drug and is the drug of choice for the oral treatment of all malaria except that caused by resistant P falciparum. Although not useful in intestinal amebiasis, it is an effective agent in the treatment of extraintestinal amebiasis, especially amebic hepatitis.

The drug is absorbed almost completely from the gastrointestinal tract and usually is administered orally. It (as the hydrochloride) is given intramuscularly when necessary to resort to parental administration. Tissues bind the drug, although not quite to the same degree of quinacrine. It is degraded in tissues to unknown products. The drug is slowly excreted in the urine with an initial half-life of 1 wk, changing to 17 days after 4 wk, then ultimately becoming months.

Appropriate doses are, oral, adults, for malaria, as a suppressive, 500 mg once a wk for 2 wk before exposure, and for therapy, initially 1 g followed by 500 mg in 6 to 8 hr, then 500 mg once a day on the 2nd and 3rd days; for extraintestinal amebiasis, 250 mg 4 times a day for 2 days followed by 250 mg twice a day for at least 2 or 3 wk; for lupus erythematosus, 250 mg twice a day for 2 wk then once a day thereafter; to suppress photoeruptions, 250 mg twice a day for 2 wk then once a day; for rheumatoid arthritis, 250 mg once a day, to be increased to as much as 750 mg a day, if necessary. Children, for malaria, as a suppressive, 8.3 mg/kg, not to exceed 500 mg, once a week, and for therapy, initially 16.7 mg/kg, not to exceed 1 g, then 8.3 mg/kg, not to exceed 00 mg, 6, 24 and 48 hr later; for extraintestinal amebiasis, 10 mg/kg, not to exceed 600 mg, every day for 3 wk.

Suppressive treatment should begin 2 wk in advance of entering into a malarious region and continue for 8 wk after departure; rapid loading for suppression can be achieved by giving the two weekly doses in a single day, 6 hr apart.

It is available in tablets of 250 and 500 mg, equivalent to 150 and 300 mg, respectively, of chloroquine base.

Chloroquine hydrochloride is 1,4-Pentanediamine, N⁴-(7-chloro-4-quinolinyl)-N¹, N¹-diethyl-, dihydrochloride; also termed Aralen Hydrochloride available from Winthrop. Its actions and uses are those of chloroquine phosphate, except that the hydrochloride lends itself better to solutions for intramuscular injection. The intramuscular route may be indicated in patients who cannot tolerate oral chloroquine.

Suitable doses are intramuscular, adults, for malaria, 200 to 250 mg, to be repeated in 6 hr, if necessary, but not to exceed 1 g in the first 24 hr; for extraintestinal amebiasis, 200 to 250 mg once a day for 10 to 12 days. Children, for malaria, 6.25 mg/kg and no more, to be repeated in 6 hr, if necessary, but not to exceed 12 mg/kg in any 24-hr period; for extraintestinal amebiasis, 7.5 mg/kg a day for 10 to 12 days. It is available in injectable forms of 250 mg/5 mL, equivalent to 200 mg/5 mL of chloroquine base.

4. Propranolol

Propranolol is also proposed for use in the present invention based upon its ability to partially reverse drug resistance in multidrug-resistant human carcinoma cells (Akiyama et al., 1988).

Propranolol is a β-adrenergic antagonist used in the treatment of hypertension and angina. The initial oral dose of propranolol is generally 40 to 80 mg per day. The dose may then be titrated upward until the optimal response is obtained. For the treatment of angina, the dose may be increased at intervals of less than 1 week, as indicated clinically. In hypertension, the full response of the blood pressure may not develop for several weeks. Typically, doses are less than 320 mg per day. Propranolol may also be administered intravenously for the management of life-threatening arrhythmias.

Propranolol is highly lipophilic and is almost complete absorbed after oral administration. However, much of the drug is metabolized by the liver during its first passage through the portal circulation; on average, only about 25% reaches the systemic circulation. In addition, there is inter-individual variation in the presystemic clearance of propranolol by the liver; this contributes to variability in plasma concentrations after oral administration of the drug (approximately 20-fold). The degree of hepatic extraction of propranolol declines as the dose is increased, and the bioavailability of propranolol may be increased by the ingestion of food and during long-term administration of the drug.

Propranolol has a large volume of distribution (4 liters/kg) and readily enters the CNS. Approximately 90% of the drug in the circulation is bound to plasma proteins. Propranolol is extensively metabolized, with most metabolites appearing in the urine.

A sustained-release formulation of propranolol has been developed to maintain therapeutic concentrations of propranolol is plasma throughout a 24-hour period. Suppression of exercise-induced tachycardia is maintained throughout the dosing interval, and patient compliance may be improved.

Propranolol hydrochloride is available in tablets that contain 10 to 90 mg of the drug for oral administration and at a concentration of 1 mg/ml for intravenous use. It is also available in sustained-release capsules (INDERAL LA) that contain 80, 120, 10 160 mg.

F. Terfenadine (Seldane)

Terfenadine (Seldane) has been proposed as a drug for restoring sensitivity to multidrug resistant cancer cells (Hait et al, 1993). The mechanism of action of terfenadine is believed to be due to inhibition of the function of biliary transport since it augments the accumulation of doxorubicin and inhibits the efflux of rhodamine 123 from MDR lines but has no effect on drug accumulation or efflux in sensitive cells.

Since terfenadine is clinically available, has numerous structural derivatives available for study, and has a relatively low toxicity profile, this drug and drugs of its class are currently preferred for use in combination with flavopiridols in the present invention.

Terfenadine is 1-Piperidinebutanol, α-[4-(1,1-dimethylethyl)phenyl]-4-(hydroxydiphenylmethyl)-, also termed Seldane, available from Merrell-Dow. Terfenadine is a peripheral distinct Hi-receptor antagonist, although chemically and pharmacologically distinct from other antihistamines. It is indicated for the relief of symptoms associated with seasonal allergic rhinitis such as sneezing, rhinorrhea, pruritus and lacrimation. It is absorbed rapidly and almost completely after oral administration; because of extensive first-pass metabolism, less than 1% reaches the systemic circulation unchanged and 97% of this is bound to plasma protein. Peak effect is observed within 1 to 2 hr; peak plasma levels range from 1.5 to 4.5 ng/mL. Plasma concentrations decline in a biphasic manner; distribution half-life is 3.5 hr and terminal elimination half-life is 16 to 23 hr.

Appropriate doses are, usual, adults and children over 12 yr, oral, 60 mg twice a day; children 6 to 12 yr, 30 to 60 mg twice a day; children 3 to 5 yr, 15 mg twice a day. It is available in tablet forms of 60 mg.

G. Ivermectin

Using mice homozygous for a disruption of the p-glycoprotein gene, Schinkel et al. (1994) conducted studies showing that such mice displayed an increased sensitivity to ivermectin. Therefore, ivermectin must normally interact with p-glycoprotein. Schinkel et al. (1994) proposed that p-glycoprotein inhibitors might be useful in selectively enhancing the access of a range of drugs to the brain.

The avermectins are a novel class of macrocyclic lactones. Ivermectin is a mixture of about 80% component B₁ₐ and 20% component B_{1b}, and is formed by selective catalytic hydrogenation of avermectin B₁. This semisynthetic agent is used extensively in veterinary medicine to treat and control a wide variety of infections caused by parasitic nematodes (roundworms) and arthropods (insects, ticks, and mites) that plague livestock and domestic animals.

In humans, ivermectin is now the drug of choice to treat and control onchocerciasis, the filarial infection responsible for river blindness. Ivermectin is effective and highly potent against at least some developmental stages of many parasitic nematodes that infect animal and man. Certain gastrointestinal nematodes that infect man are also susceptible to ivermectin. Thus, the drug appears highly effective in strongyloidiasis, ascariasis, trichuriasis, and enterobiasis; hookworms are also affected but to a lesser extent.

In humans, peak concentrations of ivermectin in plasma are achieved within 4 hours of oral administration; the half-life of the drug is about 10 hours. Animal studies reveal that only 1 to 2% of an orally administered dose of ivermectin appears in the urine; the remainder is found in the feces, nearly all as the unchanged drug.

Ivermectin is available from the Centers for Disease Control as MECTIZAN tablets, each containing 6 mg. Data indicate that a single oral dose of 0.15 to 0.20 mg/kg in adults causes a rapid and marked reduction of O. volvulus microfilaria in the skin and ocular tissues. This effect is noted within a few days and lasts for 6 to 12 months; the dose should then be repeated.

Single doses of ivermectin (0.15 to 0.20 mg/kg) given every 6 to 12 months are considered effective, safe, and practical for the control of onchocerciasis in man. Most important, such treatment results in reversal of lymphadenopathy and acute inflammatory changes in ocular tissues and arrests the development of further ocular pathology due to microfilariae. The finding that a single dose of 150 to 200 mg of ivermectin can cure human strongyloidiasis represents a significant advance, particularly because the drug is also effective against coexisting ascariasis, trichuriasis, and enterobiasis.

H. Quinidine

Akiyama et al. (1988) showed that quinidine binds to p-glycoprotein and inhibits photo-labeling. Hori et al. (1993) also showed that quinidine inhibited digoxin transport by p-glycoprotein. The ATP-dependent transport of amphiphilic cations across the hepatocyte canalicular membrane by p-glycoprotein was further shown to be inhibited by quinidine by Muller et al. (1994). Vezmar, et al. (1997) showed binding between MRP and a quinoline-based photoreactive drug (iodo-azido-amino quinoline, IAAQ). Vezmar, et al. 2000, subsequently demonstrated that MRP based multi-drug resistance may be reversed with the administration of quinidine.

Quinidine Sulfate is Cinchonan-9-ol, 6 α-methoxy, (9S)-, sulfate (2:1; salt), dihydrate, It is a Class 1A antidysrhythmic drug that decreases automaticity, membrane responsiveness, excitability and conduction velocity. It is quite effective in suppressing chronic atrial premature contractions, and in converting and protecting against recurrences of atrial fibrillation.

Quinidine is 90% absorbed by the oral route. In plasma 82% is protein-bound. The volume of distribution is 0.47 mL/g. Therapeutic plasma levels range from 3 to 6 ug/mL is reached. Elimination is 50 to 60% by hepatic biotransformation. The half-life ranges from 3 to 17 hr, but usually is 5 to 7 hr. An alkaline urine favors tubular resorption and, hence, prolongs the half-life and elevates plasma levels. Adjustments in dosage must be made when drugs (many antacids, carbonic anhydrase inhibitors) or diets that increase urine pH are use.

Suitable doses are, oral, adults, conventional capsules, initially, premature atrial and ventricular depolarizations, 200 to 300 mg every 8 hr; paroxysmal supraventricular tachycardias, 400 to 600 mg every 2 to 3 hr until conversion; atrial flutter (only after digitalization), 200 mg, adjusted upward every 2 to 3 hr until conversion; 200 mg every 2 to 3 hr for 5 to 8 doses; all maintenance, 200 to 300 mg 3 or 4 times a day; sustained-release tablets, 300 to 600 mg every 8 to 12 hr, if necessary and when tolerated; infants and children, mg/kg (or 180 mg/m²) 5 times a day. Parenteral administration is not recommended, although an injection is available.

Quinidine Gluconate is Cinchonan-9-ol,6′-methoxy-, (9S)-, mono-D gluconate (salt), also termed Quinaglute, available from Berlex, and Duraquin, available from Parke-Davis. It has the same actions, uses and toxicity as quinidine sulfate, but is preferred for intramuscular use, since it is nonirritating and stable in solution. The intravenous administration of quinidine only is warranted occasionally, but sometimes is a lifesaving measure in certain desperate conditions such as ventricular tachycardia with acute pulmonary edema or severe congestive failure. The cardiac effect may be observed in 15 to 20 min after intramuscular injection. Hypotension is frequent. It can also be used for the treatment of malaria.

Appropriate doses are, oral, adults, as extended-release tablets, 324 to 660 mg, every 6 to 12 hr; the higher doses should be used only after a trial with lower doses and clinical and laboratory reexamination and determination of plasma quinidine levels. Intramuscular, adults, initially 600 mg, followed by 400 mg at intervals as short as every 2 hr, if necessary, up to a maximum daily dose of 5 g. Intravenous, adults, 200 to 800 mg in dilute solution (20 mg/mL in isotonic dextrose injection) given at a rate of no more than 1 mL/min (20 mg/min) with continuous monitoring of the electrocardiograph and blood pressure. It is available in injectable dosage forms of 800 mg/10 mL; and as extended-release tablets of 342 and 330 mg.

Quinidine polygalacturonate, also termed cardiaquin, is available from Purdue-Frederick. The actions, uses and general toxicity are those of quinidine sulfate, except that it is not used in attempted conversion of ventricular dysrhythmias and it causes a lesser incidence and severity of gastrointestinal side effects and hence is gaining preference for oral use.

Appropriate doses are oral, adults, initially 275 to 825 mg for 3 or 4 doses at 3- to 4-hr intervals, after which upward adjustments in increments of 137.5 to 275 mg may be made every third or fourth dose until the therapeutic end point is reached or toxicity supervenes, then 275 mg 2 or 3 times a day for maintenance; children, 8 to 25 mg/kg or 247.5 mg/m² 3 times a day according to need and tolerance. It is available in tablet dosage forms of 275 mg, equivalent to 200 mg of quinidine sulfate.

I. Monoclonal Antibodies

A variety of monoclonal antibodies have been developed against members of the ABC protein family and specifically biliary transport proteins. Exemplary monoclonal antibodies are described in: p-glycoptrotein-MRK16 and MH162 ( Hamada et al. 1990) MH171 (Ariyoshi et al. 1992), UIC2 (Mechetner & Roninson 1992); MRP3-(Kool, 1999); MRP1- MRPr1 (Gutmann, 1999), QCRL-1 and QCRL-3 (Filipits, 1999).

J. Reduced Folates

A comparison of the transport properties across the bile canalicular membrane in normal and mutant rats, whose cMOAT function is hereditarily defective, has shown that the physiologic role of cMOAT is to excrete LTC4, bilirubin glucuronides, 171-estradiol-170-D-glucuronide, and reduced folates (Suzuki, 1998). The folates are molecules that act as carriers of one carbon units in intermediary metabolism. The folates comprise residues of p-aminobenzoate, glutamate and a substituted pteridine. Folates are not synthesized by mammals, but must be must obtained as the vitamin tetrahydrofolate from the diet or from intestinal microorganisms.

Kushara, et al. 1999, determined that reduced folate derivatives are endogenous substrates for cMOAT in rats. They are the first endogenous substrates for cMOAT that do not contain glutathione, glucuronide, or sulfate moieties. It is envisioned that folate and folate derivatives may function in the context of the instant invention to regulate the activity of cMOAT.

K. Genistein

Genistein is an isoflavonoid derived from soy products that inhibits protein-tyrosine kinase and topoisomerase-II (DNA topoisomerase (ATP-hydrolysing)) activity. Genistein is used therapeutically as an antineoplastic and antitumor agent and has been shown to induce G2 phase arrest in human and murine cell lines.

Jager, et al., 1998 showed that genistein and its metabolites are substrates of cMOAT, which partially explains the inhibition of anion secretion by genistein. It is envisioned that genistein may function in the context of the instant invention to regulate the activity of cMOAT.

L. Probenecid

Probenecid inhibits transport of organic acids across epithelial barriers (Overbosch, 1988). This is most important in the renal tubule, where probenecid inhibits tubular secretion of many drugs and metabolites, thereby reducing their concentration in urine and raising their plasma concentration. Probenecid is good for keeping high plasma levels of substances that do not have a high glomerular filtration, since it only inhibits tubular secretion.

Probenecid is completely absorbed after oral administration. Peak concentrations in plasma are reached in 2 to 4 hours. The half life is dose dependent and varies form 5 to 8 hours over the therapeutic range (Goodman, 1990). 85% to 90% of the drug is bound to plasma albumin (Overbosch, 1988), and it is secreted by the proximal tubule. The unbound portion gains access to glomerular filtrate.

The recommended dosage for adults is 0.25 g twice a day for 1 week, followed by 0.5 g twice a day thereafter. In patients suffering of renal impairment a daily dosage of 1 g may be adequate. If necessary, however, the daily weeks by increments of 0.5 g (within tolerance, and usually not beyond 2 g daily).

M. Ritonavir

Ritonavir is a protease inhibitor commonly used alone or in combination to treat HIV infection. MRP1 and MDR1 have been linked to the control of ritonavir concentration in HIV-targeted cells (Zhang, et al. 2000).

Recommended dosage is up to 600 mg twice a day. It is recommended that this dose be assumed slowly over a few days when first starting the drug. The recommended starting dose is 300 mg twice a day, with progression to 400 mg twice a day after a few days, continuing until the recommended dose of 600 mg twice a day is reached.

N. Interferons

The interferons (IFNs) are a group of cytokines, which in addition to their antiviral activity are capable of modulating a variety of cellular responses. One such prominent effect of IFNs is their potent antimitogenic action, which can be observed both on malignant and non-malignant cells of many different origins. IFNs are also used in the clinic, mainly in malignant and viral diseases, and their cell growth-inhibitory effect has been suggested to be of major importance in their antitumour and antiviral action.

Multidrug resistance mechanisms are responsive to cytokines and immunomodulators, specifically interferon-alpha (IFN; Savas, et al. 1999). In addition, evidence suggests that IFNα is involved in the up-regulation of multidrug resistance (MDR) gene expression, and that the MDR gene product, p-glycoprotein. facilitates the transport of several cytokines, some of which have been implicated in mediating tumor antiproliferative effects. (Frank et al., 1999). Athough IFN-alpha is not a substrate of p-glycoprotein, it significantly increased the ability of substrates to bind (Kang, 1994)

O. Additional Substrates and Inhibitors of Biliary Transport

A number of additional substrates for members of the ABC protein family are disclosed in the art. One of ordinary skill would recognize the relevance of the disclosed substrates or inhibitors in the context of the instant invention. Examples of specific substrates and inhibitors are set forth in Table 2.

TABLE 2

| Substrate/Inhibitor | Biliary Transport Protein |
|---|---|
| [(3)H]2,4-Dinitrophenyl-S-glutathione | MRP1 and cMOAT (Hirohashi, |
| (DNP-SG) | et al., 2000) |
| [(3)H]17beta-estradiol 17-beta-D- | MRP1 and cMOAT (Hirohashi, |
| glucuronide (E(2)17betaG) | et al., 2000) |
| Dipyridamole | MRP (Curtin, et al. 1999) |
| 7-chloro-4-nitrobenz-2-oxa-1,3-diazole | MRP (Benderra, et al 1999) |
| Buthionine sulphoximine | MRP (Benderra, et al 1999) |
| MK751 | MRP1 (Gutmann, et al. 1999) |
| Leukotriene C4 (LTC4) | MRP1 (Sakamoto et al. 1999). |
| Bromosulfophthalein (BSP) | cMOAT (Pang, et al. 1998) |
| Enalapril | cMOAT (Pang, et al. 1998) |
| CRC 220 | cMOAT (Pang, et al. 1998) |
| Taurocholate (TCA) | cMOAT (Pang, et al. 1998) |
| N-acetylcysteine and cysteine | cMOAT (Zallups, 1998) |
| [3H]Temocaprilat | cMOAT (Ishizuka, et al. 1998) |
| Estradiol-17beta-D-glucuronide | cMOAT (Ishizuka, et al. 1998) |
| Dibromosulphophthalein | cMOAT (Ishizuka, et al. 1998) |

P. NSAID

Non-steroidal anti-inflammatory drugs are a large group of anti-inflammatory agents thats primary mechanism of action involves the inhibition of the production of prostaglandins. Examples of common NSAID's include: indomethacin, sulindac, tolmetin, acemetacin, zomepirac, mefenamic acid, ibuprofen, ketoprofen, piroxicam, naproxen, sulindac, aspirin, choline subsalicylate, diflunisal, fenoprofen, meclofenamate, salsalate, tolmetin, etodolac, nabumetone, oxaprozin, rofecoxib, celocoxib, diflunisal, salsalate, ketorolac, tolectin, clinoril, mefenamic acid, fenoprofen calcium, meclofenamate sodium, piroxicam, diclofenac and magnesium salicylate.

NSAID administration has been demonstrated to increase chemotherapeutic cytotoxicity in cell lines in which multidrug resistance is due to overexpression of the multidrug resistance-associated protein MRP (Duffy, et al. 1998). Positive NSAIDs, i.e. indomethacin, sulindac, tolmetin, acemetacin, zomepirac and mefenamic acid, are among the more potent inhibitors of [3H]-LTC4 transport into inside-out plasma membrane vesicles prepared from MRP-expressing cells, of doxorubicin efflux from preloaded cells and of glutathione-S-transferase activity (Duffy, et al. 1998). In addition, indomethacin is a known cyclo-oxygenase and glutathione-S-transferase inhibitor as well as a modulator of anion transport increased accumulation and blocked efflux of BCECF in MRP-expressing murine and human cells (Draper, et al 1997).

Q. Nucleic Acids

The present invention involves nucleic acids, including UGT-encoding nucleic acids, nucleic acids identical or complementary to all or part of the sequence of a UGT gene as well as nucleic acids constructs and primers. Such UGT-encoding nucleic acids, include, but are not limited to, the following UGT molecules, UGT1A1, UGT1A3, UGT1A4, UGT1A6, UGT1A8, UGT1A10, UGT2B4, UGT2B7, UGT2B15 and UGT2B17. While in some embodiments UGT1A9 nucleic acids are discussed, it is contemplated that any other UGT-encoding nucleic acid may be employed as UGT1A9 nucleic acid discussed below.

The present invention involves nucleic acids, also including ABC transport protein-encoding nucleic acids, nucleic acids identical or complementary to all or part of the sequence of a ABC gene as well as nucleic acids constructs and primers. Such ABC-encoding nucleic acids, include, but are not limited to, the following the ABC subfamilies such as, ABC1 (also called subfamily A and includes members identified by the symbols ABCA1 (also named variously as ABC1, TGD, HDLDT1, CERP), ABCA2, ABCA3 (also named variously as ABC-C, EST 111653), ABCA4 (also named variously as ABCR, RP-19 ABC10, FFM, STGD1, STGD), ABCA5, ABCA6, ABCA7, ABCA8, ABCA9, ABCA10, ABCA11, ABCA12, ABCA13, ABCA14); MDR/TAP (also called subfamily B and includes members identified by the symbols ABCB1 (also named variously as PGY1, MDR1, PGP, GP170), ABCB2 (also named variously as TAP1, PSF1, RING4, ABC17, APT1, D6S114E), ABCB3 (also named variously as TAP2, PSF2, RING11, ABC18, D6S17E), ABCB4 (also named variously as PGY3, MDR2, MDR3, ABC21), ABCB5, ABCB6, ABCB7, ABCB8, ABCB9 (also named variously as TAPL), ABCB10, ABCB1 l(also named variously as BSEP, SPGP, PFIC2, PGY4, ABC16); CFTR/MRP (also called subfamily C and includes members identified by the symbols ABCC1 (also named variously as MRP1, MRP), ABCC2 (also named variously as CMOAT), ABCC3 (also named variously as MRP3), ABCC4 (also named variously as MRP4), ABCC5 (also named variously as MRP5), ABCC6 (also named variously as MRP6), ABCC7 (also named variously as CFTR), ABCC8 (also named variously as SUR1), ABCC9 (also named variously as SUR2), ABCC10, ABCC11, ABCC12, ABCC13); ALD (also called subfamily D and includes members identified by the symbols ABCD1, ABCD2, ABCD3, ABCD4); OABP (also called subfamily E and includes members identified by the symbols ABCE1); GCN20 (also called subfamily F and includes members identified by the symbols ABCF1, ABCF2, ABCF3); White (also called subfamily G and includes members identified by the symbols ABCG1 (also named variously as ABC8, White), ABCG2 (also named variously as BCRP1, MXR1, ABCP), ABCG4, ABCG5, ABCG8). See also http://www.med.rug.nl/mdl/humanabc.htm (incorporated herein by reference) for details and links to other ABC-encoding nucleic acids. As set forth above, although the general description in this section describes UGT1A9 nucleic acids, it is contemplated that an ABC-encoding nucleic acid may be employed in place of the UGT1A9 nucleic acid discussed below. Some of these ABC transporter protein-encoding nucleic acids are described by the following GenBank Accession numbers, NM005502, XM005567, AF178941, NM001089, XM007924, NM000350, XM001290, NM0191, NM007168, AC005922, AC005495, NM000927, XM004598, NM000593, NM000544, NM000443, NM005689, NM004299, NM007188, NM019624, NM012089, NM003742, NM004996, NM000392, NM003786, NM005845, NM005688, NM001171, NM00492, NM000352, NM005691, AK00002, NM000033, NM005164, NM002858, NM005050, NM002940, NM001090, AC000384, AF320293, AF320294, and NM004827.

The present invention concerns polynucleotides or nucleic acid molecules relating to the UGT1A9 gene and its gene product UGT1A9. These polynucleotides or nucleic acid molecules are isolatable and purifiable from mammalian cells. It is contemplated that an isolated and purified UGT1A9 nucleic acid molecule, that is a nucleic acid molecule related to the UGT1A9 gene product, may take the form of RNA or DNA. As used herein, the term “RNA transcript” refers to an RNA molecule that is the product of transcription from a DNA nucleic acid molecule. Such a transcript may encode for one or more polypeptides.

As used in this application, the term “polynucleotide” refers to a nucleic acid molecule, RNA or DNA, that has been isolated free of total genomic nucleic acid. Therefore, a “polynucleotide encoding UGT1A9” refers to a nucleic acid segment that contains UGT1A9 coding sequences, yet is isolated away from, or purified and free of, total genomic DNA and proteins. When the present application refers to the function or activity of a UGT1A9-encoding polynucleotide or nucleic acid, it is meant that the polynucleotide encodes a molecule that has the ability to glucuronidate a substrate, such as flavopiridol or an analog thereof.

The term “cDNA” is intended to refer to DNA prepared using RNA as a template. The advantage of using a cDNA, as opposed to genomic DNA or an RNA transcript is stability and the ability to manipulate the sequence using recombinant DNA technology (See Sambrook, 1989; Ausubel, 1996). There may be times when the full or partial genomic sequence is preferred. Alternatively, cDNAs may be advantageous because it represents coding regions of a polypeptide and eliminates introns and other regulatory regions.

It also is contemplated that a given UGT1A9-encoding nucleic acid or UGT1A9 gene from a given cell may be represented by natural variants or strains that have slightly different nucleic acid sequences but, nonetheless, encode a UGT1A9 polypeptide; a human UGT1A9 polypeptide is a preferred embodiment. Consequently, the present invention also encompasses derivatives of UGT1A9 with minimal amino acid changes, but that possess the same activity.

The term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid molecule encoding UGT1A9 or a UGT1A9 gene may comprise a contiguous nucleic acid sequence of the following lengths: at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000 or more nucleotides, nucleosides, or base pairs. Such sequences may be identical or complementary to SEQ ID NO:1 (UGT1A9 mRNA sequence; GenBank Accession No. NM_{—}021027) and SEQ ID NO:3 (UGT1A9 cDNA and mRNA sequence; GenBank Accession No. AF056188) and primers to amplify or sequence all or part of SEQ ID NO:1 or SEQ ID NO:3.

In some embodiments, genetic polymorphisms in UGT1A9 are relevant. As used herein, a “single nucleotide polymorphism” (SNP) refers to an addition, deletion, or substitution of a single nucleotide at a site in a nucleic acid molecule; it reflects the occurrence of genetically determined variant forms of a nucleic acid sequence at a frequency where the rarest could not be maintained by recurrent mutation alone. In some instances, a polymorphism in a sequence results in a change that affects the activity, expression, or stability of a transcript or polypeptide encoded by the sequence. Thus, in some embodiments of the present invention, a polymorphism in a UGT1A9 gene results in a change in effective UGT1A9 enzyme activity or the level of UGT1A9 protein or transcript expression.

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid segment as originally isolated, and does not exclude genes or coding regions later added to the segment by human manipulation.

In particular embodiments, the invention concerns isolated DNA segments and recombinant vectors incorporating DNA sequences that encode a UGT1A9 protein, polypeptide or peptide that includes within its amino acid sequence a contiguous amino acid sequence in accordance with, or essentially as set forth in, SEQ ID NO:2, or SEQ ID NO:4, corresponding to the UGT1A9 designated “human UGT1A9.”

The term “a sequence essentially as set forth in SEQ ID NO:2 or SEQ ID NO:4” means that the sequence substantially corresponds to a portion of SEQ ID NO:2 or SEQ ID NO:4 and has relatively few amino acids that are not identical to, or a biologically functional equivalent of, the amino acids of SEQ ID NO:2 or SEQ ID NO:4.

The term “biologically functional equivalent” is well understood in the art and is further defined in detail herein. Accordingly, sequences that have about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, and any range derivable therein, such as, for example, about 70% to about 80%, and more preferably about 81% and about 90%; or even more preferably, between about 91% and about 99%; of amino acids that are identical or functionally equivalent to the amino acids of SEQ ID NO:2 or SEQ ID NO:4 will be sequences that are “essentially as set forth in SEQ ID NO:2 or SEQ ID NO:4” provided the biological activity of the protein is maintained. In particular embodiments, the biological activity of a UGT1A9 protein, polypeptide or peptide, or a biologically functional equivalent, comprises catalyzing the glucuronidation of a substrate such as flavopiridol or an analog thereof. In certain other embodiments, the invention concerns isolated DNA segments and recombinant vectors that include within their sequence a nucleic acid sequence essentially as set forth in SEQ ID NO:1 or SEQ ID NO:3. The term “essentially as set forth in SEQ ID NO:1” is used in the same sense as described above and means that the nucleic acid sequence substantially corresponds to a portion of SEQ ID NO:1 or SEQ ID NO:3 and has relatively few codons that are not identical, or functionally equivalent, to the codons of SEQ ID NO:1 or SEQ ID NO:3. Again, DNA segments that encode proteins, polypeptide or peptides exhibiting UGT1A9 activity will be most preferred.

In particular embodiments, the invention concerns isolated nucleic acid segments and recombinant vectors incorporating DNA sequences that encode UGT1A9 polypeptides or peptides that include within its amino acid sequence a contiguous amino acid sequence in accordance with, or essentially corresponding to UGT1A9 polypeptides.

The nucleic acid segments used in the present invention, regardless of the length of the coding sequence itself, may be combined with other DNA or RNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode UGT1A9. A “heterologous” sequence refers to a sequence that is foreign or exogenous to the remaining sequence. A heterologous gene refers to a gene that is not found in nature adjacent to the sequences with which it is now placed.

In a non-limiting example, one or more nucleic acid constructs may be prepared that include a contiguous stretch of nucleotides identical to or complementary to all or part of a UGT1A9 gene. A nucleic acid construct may comprise at least 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 20,000, 30,000, 50,000, 100,000, 250,000, about 500,000, 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, about 97001, about 1,001, about 1002, about 50,001, about 50,002, about 750,001, about 750,002, about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc.

The nucleic acid segments used in the present invention encompass biologically functional equivalent UGT1A9 proteins and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein or to test mutants in order to examine DNA binding activity at the molecular level.

Certain embodiments of the present invention concern various nucleic acids, including vectors, promoters, therapeutic nucleic acids, and other nucleic acid elements involved in transformation and expression in cells. In certain aspects, a nucleic acid comprises a wild-type or a mutant nucleic acid. In particular aspects, a nucleic acid encodes for or comprises a transcribed nucleic acid.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. A “gene” refers to coding sequence of a gene product, as well as introns and the promoter of the gene product. In addition to the UGT1A9 gene, other regulatory regions such as enhancers for UGT1A9 are contemplated as nucleic acids for use with compositions and methods of the claimed invention.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss”, a double stranded nucleic acid by the prefix “ds”, and a triple stranded nucleic acid by the prefix “ts.”

In particular aspects, a nucleic acid encodes a protein, polypeptide, or peptide. In certain embodiments, the present invention concerns novel compositions comprising at least one proteinaceous molecule. As used herein, a “proteinaceous molecule,” “proteinaceous composition,” “proteinaceous compound,” “proteinaceous chain,” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

1. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

2. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference). In preferred aspects, a nucleic acid is a pharmacologically acceptable nucleic acid. Pharmacologically acceptable compositions are known to those of skill in the art, and are described herein.

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells.

In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

3. Nucleic Acid Segments

In certain embodiments, the nucleic acid is a nucleic acid segment. As used herein, the term “nucleic acid segment,” are fragments of a nucleic acid, such as, for a non-limiting example, those that encode only part of a peptide or polypeptide sequence. Thus, a “nucleic acid segment” may comprise any part of a gene sequence, including from about 2 nucleotides to the full length of a peptide or polypeptide encoding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

4. Nucleic Acid Complements

The present invention also encompasses a nucleic acid that is complementary to a nucleic acid. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule. In preferred embodiments, a complement is an antisense nucleic acid used to reduce expression (e.g., translation) of a RNA transcript in vivo.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. However, in some antisense embodiments, completely complementary nucleic acids are preferred.

5. Vectors Encoding UGT1A9

The present invention encompasses the use of vectors to encode for UGT1A9 and candidate modulators of UGT1A9. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1996, both incorporated herein by reference.

The term “expression vector” or “expression construct” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5 non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the nucleic acid segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level exp ression of the introduced DNA segment, such as is advantageous in the large-scale production of recombinant proteins and/or peptides. The promoter may be heterologous or exogenous, for example, a non-UGT1A9 promoter with respect to UGT1A9 encoding sequence. In some examples, a prokaryotic promoter is employed for use with in vitro transcription of a desired sequence. Prokaryotic promoters for use with many commercially available systems include T7, T3, and Sp6.

Table 3 lists several elements/promoters that may be employed, in the context of the present invention, to regulate the expression of a gene. This list is not intended to be exhaustive of all the possible elements involved in the promotion of expression but, merely, to be exemplary thereof. Table 4 provides examples of inducible elements, which are regions of a nucleic acid sequence that can be activated in response to a specific stimulus.

TABLE 3
Promoter and/or Enhancer

| Promoter/Enhancer | References |
|---|---|
| Immunoglobulin Heavy | Banerji et al., 1983; Gilles et al., 1983; |
| Chain | Grosschedl et al., 1985; Atchinson et al., |
| | 1986, 1987; Imler et al., 1987; Weinberger |
| | et al., 1984; Kiledjian et al., 1988; Porton |
| | et al.; 1990 |
| Immunoglobulin Light | Queen et al., 1983; Picard et al., 1984 |
| Chain | |
| T-Cell Receptor | Luria et al., 1987; Winoto et al., 1989; |
| | Redondo et al.; 1990 |
| HLA DQ * and/or DQ | Sullivan et al., 1987 |
| -Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| -Actin | Kawamoto et al., 1988; Ng et al.; 1989 |
| Muscle Creatine Kinase | Jaynes et al., 1988; Horlick et al., 1989; |
| (MCK) | Johnson et al., 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, |
| | 1990 |
| -Fetoprotein | Godbout et al., 1988; Campere et al., 1989 |
| -Globin | Bodine et al., 1987; Perez-Stable et al., 1990 |
| -Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsh et al., 1990 |
| Molecule (NCAM) | |
| ₁-Antitrypain | Latimer et al., 1990 |
| H2B (TH2B) Histone | Hwang et al., 1990 |
| Mouse and/or Type I | Ripe et al., 1989 |
| Collagen | |
| Glucose-Regulated Proteins | Chang et al., 1989 |
| (GRP94 and GRP78) | |
| Rat Growth Hormone | Larsen et al., 1986 |
| Human Serum Amyloid A | Edbrooke et al., 1989 |
| (SAA) | |
| Troponin I (TN I) | Yutzey et al., 1989 |
| Platelet-Derived Growth | Pech et al., 1989 |
| Factor (PDGF) | |
| Duchenne Muscular | Klamut et al., 1990 |
| Dystrophy | |
| SV40 | Banerji et al., 1981; Moreau et al., 1981; |
| | Sleigh et al., 1985; Firak et al., 1986; Herr |
| | et al., 1986; Imbra et al., 1986; Kadesch et |
| | al., 1986; Wang et al., 1986; Ondek et al., |
| | 1987; Kuhl et al., 1987; Schaffner et al., |
| | 1988 |
| Polyoma | Swartzendruber et al., 1975; Vasseur et al., |
| | 1980; Katinka et al., 1980, 1981; Tyndell et |
| | al., 1981; Dandolo et al., 1983; de Villiers |
| | et al., 1984; Hen et al., 1986; Satake et al., |
| | 1988; Campbell and/or Villarreal, 1988 |
| Retroviruses | Kriegler et al., 1982, 1983; Levinson et al., |
| | 1982; Kriegler et al., 1983, 1984a, b, 1988; |
| | Bosze et al., 1986; Miksicek et al., 1986; |
| | Celander et al., 1987; Thiesen et al., 1988; |
| | Celander et al., 1988; Chol et al., 1988; |
| | Reisman et al., 1989 |
| Papilloma Virus | Campo et al., 1983; Lusky et al., 1983; |
| | Spandidos and/or Wilkie, 1983; Spalholz |
| | et al., 1985; Lusky et al., 1986; Cripe et al., |
| | 1987; Gloss et al., 1987; Hirochika et al., |
| | 1987; Stephens et al., 1987; Glue et al., 1988 |
| Hepatitis B Virus | Bulla et al., 1986; Jameel et al., 1986; Shaul |
| | et al., 1987; Spandau et al., 1988; Vannice et |
| | al., 1988 |
| Human Immunodeficiency | Muesing et al., 1987; Hauber et al., 1988; |
| Virus | Jakobovits et al., 1988; Feng et al., 1988; |
| | Takebe et al., 1988; Rosen et al., 1988; |
| | Berkhout et al., 1989; Laspia et al., 1989; |
| | Sharp et al., 1989; Braddock et al., 1989 |
| Cytomegalovirus (CMV) | Weber et al., 1984; Boshart et al., 1985; |
| | Foecking et al., 1986 |
| Gibbon Ape Leukemia | Holbrook et al., 1987; Quinn et al., 1989 |
| Virus | |

TABLE 4
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa et |
| | | al., 1987, Karin et al., |
| | | 1987; Angel et al., |
| | | 1987b; McNeall et al., |
| | | 1989 |
| MMTV (mouse | Glucocorticoids | Huang et al., 1981; Lee |
| mammary tumor virus) | | et al., 1981; Majors et |
| | | al., 1983; Chandler et al., |
| | | 1983; Lee et al., 1984; |
| | | Ponta et al., 1985; Sakai |
| | | et al., 1988 |
| -Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| -2-Macroglobulin | IL-6 | Kunz et al., 1989 |
| Vimentin | Serum | Rittling et al., 1989 |
| MHC Class I Gene H- | Interferon | Blanar et al., 1989 |
| 2b | | |
| HSP70 | ElA, SV40 Large T | Taylor et al., 1989, |
| | Antigen | 1990a, 1990b |
| Proliferin | Phorbol Ester-TPA | Mordacq et al., 1989 |
| Tumor Necrosis | PMA | Hensel et al., 1989 |
| Factor | | |
| Thyroid Stimulating | Thyroid Hormone | Chatterjee et al., 1989 |
| Hormone Gene | | |

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (WU et al., 1997), human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

C. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, herein incorporated by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

For expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, the cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

6. Host Cells

As used herein, the terms “cell,” “cell line,” and “cell culture” may be used interchangeably. All of these terms also include their progeny, which refers to any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. A “recombinant host cell” refers to a host cell that carries a recombinant nucleic acid, i.e. a nucleic acid that has been manipulated in vitro or that is a replicated copy of a nucleic acid that has been so manipulated.

A host cell may be derived from prokaryotes or eukaryotes, depending upon whether the desired result is replication of the vector, expression of part or all of the vector-encoded nucleic acid sequences, or production of infectious viral particles. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5, JM109, and KC8, as well as a number of commercially available bacterial hosts such as SURE® Competent Cells and SOLOPACK Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either an eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

7. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM from CLONTECH®.

Other examples of expression systems include STRATAGENE®'S COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. The Tet-On™ and Tet-Off™ systems from CLONTECH® can be used to regulate expression in a mammalian host using tetracycline or its derivatives. The implementation of these systems is described in Gossen et al., 1992 and Gossen et al., 1995, and U.S. Pat. No. 5,650,298, all of which are incorporated by reference.

INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

8. Viral Vectors

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986).

The retroviruses are a group of single-stranded RNA viruses characterized by an ability to convert their RNA to double-stranded DNA in infected cells; they can also be used as vectors. Other viral vectors may be employed as expression constructs in the present invention. Vectors derived from viruses such as vaccinia virus (Ridgeway, 1988; Baichwal and Sugden, 1986; Coupar et al, 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

9. Nucleic Acid Detection

In some embodiments the invention concerns identifying polymorphisms in UGT1A9, correlating genotype to phenotype, wherein the phenotype is lowered UGT1A9 activity or expression, and then identifying such polymorphisms in patients who have or will be given flavopiridol. Thus, the present invention involves assays for identifying polymorphisms and other nucleic acid detection methods. Nucleic acids, therefore, have utility as probes or primers for embodiments involving nucleic acid hybridization. They may be used in diagnostic or screening methods of the present invention. Detection of nucleic acids encoding UGT1A9, as well as nucleic acids involved in the expression or stability of UGT1A9 polypeptides or transcripts, are encompassed by the invention. General methods of nucleic acid detection methods are provided below, followed by specific examples employed for the identification of polymorphisms, including single nucleotide polymorphisms (SNPs).

a. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide. For example, under highly stringent conditions, hybridization to filter-bound DNA may be carried out in 0.5 M NaHPO₄, 7% sodium dodecyl sulfate (SDS), 1 mM EDTA at 65° C., and washing in 0.1×SSC/0.1% SDS at 68° C. (Ausubel F. M. et al., Eds., 1989, Current Protocols in Molecular Biology, Vol. I, Green Publishing Associates, Inc., and John Wiley & sons, Inc., New York, at p. 2.10.3).

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Under low stringent conditions, such as moderately stringent conditions the washing may be carried out for example in 0.2×SSC/0.1% SDS at 42° C. (Ausubel et al., 1989, supra). Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

b. Amplification of Nucleic Acids

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to SEQ ID NO:1 and fragments thereof are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintgraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA) (described in further detail below), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation

Other nucleic acid amplification procedures include transcription-based amplification systems (TAS), including nucleic acid sequence based amplification (NASBA) and 3SR (Kwoh et al, 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

C. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

d. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investgators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

e. Specific Examples of SNP Screening Methods

Spontaneous mutations that arise during the course of evolution in the genomes of organisms are often not immediately transmitted throughout all of the members of the species, thereby creating polymorphic alleles that co-exist in the species populations.

Often polymorphisms are the cause of genetic diseases. Several classes of polymorphisms have been identified. For example, variable nucleotide type polymorphisms (VNTRs), arise from spontaneous tandem duplications of di- or trinucleotide repeated motifs of nucleotides. If such variations alter the lengths of DNA fragments generated by restriction endonuclease cleavage, the variations are referred to as restriction fragment length polymorphisms (RFLPs). RFLP s are been widely used in human and animal genetic analyses.

Another class of polymorphisms are generated by the replacement of a single nucleotide. Such single nucleotide polymorphisms (SNPs) rarely result in changes in a restriction endonuclease site. Thus, SNPs are rarely detectable restriction fragment length analysis. SNPs are the most common genetic variations and occur once every 100 to 300 bases and several SNP mutations have been found that affect a single nucleotide in a protein-encoding gene in a manner sufficient to actually cause a genetic disease. SNP diseases are exemplified by hemophilia, sickle-cell anemia, hereditary hemochromatosis, late-onset alzheimer disease etc.

In context of the present invention, polymorphic mutations that affect the activity and/or levels of the UGT1A9 gene products, which are responsible for the glucuronidation of flavopiridol and other chemotherapeutic and xenobiotic agents, will be determined by a series of screening methods. One set of screening methods is aimed at identifying SNPs that affect the activity and/or level of the UGT1A9 gene products in in vitro assays. The other set of screening methods will then be performed to screen an individual for the occurrence of the SNPs identified above. To do this, a sample (such as blood or other bodily fluid or tissue sample) will be taken from a patient for genotype analysis. The presence or absence of SNPs will determine the ability of the screened individuals to metabolize flavopiridol and other chemotherapeutic agents that are metabolized by the UGTB27 gene products. According to methods provided by the invention, these results will be used to adjust and/or alter the dose of flavopiridol or other agent administered to an individual in order to reduce drug side effects.

SNPs can be the result of deletions, point mutations and insertions and in general any single base alteration, whatever the cause, can result in a SNP. The greater frequency of SNPs means that they can be more readily identified than the other classes of polymorphisms. The greater uniformity of their distribution permits the identification of SNPs “nearer” to a particular trait of interest. The combined effect of these two attributes makes SNPs extremely valuable. For example, if a particular trait (e.g., inability to efficiently metabolize flavopiridol) reflects a mutation at a particular locus, then any polymorphism that is linked to the particular locus can be used to predict the probability that an individual will be exhibit that trait.

Several methods have been developed to screen polymorphisms and some examples are listed below. SNPs relating to glucuronidation of chemotherapeutic agents can be characterized by the use of any of these methods or suitable modification thereof. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or any other biochemical interpretation.

i) DNA Sequencing

The most commonly used method of characterizing a polymorphism is direct DNA sequencing of the genetic locus that flanks and includes the polymorphism. Such analysis can be accomplished using either the “dideoxy-mediated chain termination method,” also known as the “Sanger Method” (Sanger, F., et al., 1975) or the “chemical degradation method,” also known as the “Maxam-Gilbert method” (Maxam, A. M., et al., 1977). Sequencing in combination with genomic sequence-specific amplification technologies, such as the polymerase chain reaction may be utilized to facilitate the recovery of the desired genes (Mullis, K. et al., 1986; European Patent Appln. 50,424; European Patent Appln. 84,796, European Patent Application 258,017, European Patent Appln. 237,362; European Patent Appln. 201,184; U.S. Pat. Nos. 4,683,202; 4,582,788; and 4,683,194), all of the above incorporated herein by reference.

ii) Exonuclease Resistance

Other methods that can be employed to determine the identity of a nucleotide present at a polymorphic site utilize a specialized exonuclease-resistant nucleotide derivative (U.S. Pat. No. 4,656,127). A primer complementary to an allelic sequence immediately 3′-to the polymorphic site is hybridized to the DNA under investigation. If the polymorphic site on the DNA contains a nucleotide that is complementary to the particular exonucleotide-resistant nucleotide derivative present, then that derivative will be incorporated by a polymerase onto the end of the hybridized primer. Such incorporation makes the primer resistant to exonuclease cleavage and thereby permits its detection. As the identity of the exonucleotide-resistant derivative is known one can determine the specific nucleotide present in the polymorphic site of the DNA.

iii) Microsequencing Methods

Several other primer-guided nucleotide incorporation procedures for assaying polymorphic sites in DNA have been described (Komher, J. S. et al., 1989; Sokolov, B. P., 1990; Syvanen 1990; Kuppuswamy et al., 1991; Prezant et al., 1992; Ugozzoll, L. et al., 1992; Nyren et al., 1993). These methods rely on the incorporation of labeled deoxynucleotides to discriminate between bases at a polymorphic site. As the signal is proportional to the number of deoxynucleotides incorporated, polymorphisms that occur in runs of the same nucleotide result in a signal that is proportional to the length of the run (Syvanen et al.,1993).

iv) Extension in Solution

French Pat. No. 2,650,840 and PCT Application No. W091/02087 discuss a solution-based method for determining the identity of the nucleotide of a polymorphic site. According to these methods, a primer, complementary to allelic sequences immediately 3′-to a polymorphic site is used. The identity of the nucleotide of that site is determined using labeled dideoxynucleotide derivatives which are incorporated at the end of the primer if complementary to the nucleotide of the polymorphic site.

v) Genetic Bit Analysis or Solid-Phase Extension

PCT Appln. No. 92/15712 describes a method that uses mixtures of labeled terminators and a primer that is complementary to the sequence 3′ to a polymorphic site. The labeled terminator that is incorporated is complementary to the nucleotide present in the polymorphic site of the target molecule being evaluated and is thus identified. Here the primer or the target molecule is immobilized to a solid phase.

vi) Oligonucleotide Ligation Assay (OLA)

This is another solid phase method that uses different methodology (Landegren et al., 1988). Two oligonucleotides, capable of hybridizing to abutting sequences of a single strand of a target DNA are used. One of these oligonucleotides is biotinylated while the other is detectably labeled. If the precise complementary sequence is found in a target molecule, the oligonucleotides will hybridize such that their termini abut, and create a ligation substrate. Ligation permits the recovery of the labeled oligonucleotide by using avidin. Other nucleic acid detection assays, based on this method, combined with PCR are also described (Nickerson et al., 1990). Here PCR is used to achieve the exponential amplification of target DNA, which is then detected using the OLA.

vii) Ligase/Polymerase-Mediated Genetic Bit Analysis

U.S. Pat. No. 5,952,174 describes a method that also involves two primers capable of hybridizing to abutting sequences of a target molecule. The hybridized product is formed on a solid support to which the target is immobilized. Here the hybridization occurrs such that the primers are separated from one another by a space of a single nucleotide. Incubating this hybridized product in the presence of a polymerase, a ligase, and a nucleoside triphosphate mixture containing at least one deoxynucleoside triphosphate allows the ligation of any pair of abutting hybridized oligonucleotides. Addition of a ligase results in two events required to generate a signal, extension and ligation. This provides a higher specificity and lower “noise” than methods using either extension or ligation alone and unlike the polymerase-based assays, this method enhances the specificity of the polymerase step by combining it with a second hybridization and a ligation step for a signal to be attached to the solid phase.

viii) Other Methods To Detect SNPs

Several other specific methods for SNP detection and identification are presented below and may be used as such or with suitable modifications in conjunction with identifying polymorphisms of the UGT!A9 genes in the present invention. Several other methods are also described on the SNP web site of the NCBI at http://www.ncbi.nlm.nih.gov/SNP, incorporated herein by reference.

The VDA-assay utilizes PCR amplification of genomic segments by long PCR methods using TaKaRa LA Taq reagents and other standard reaction conditions. The long amplification can amplify DNA sizes of about 2,000-12,000 bp. Hybridization of products to variant detector array (VDA) can be performed by a Affymetrix High Throughput Screening Center and analyzed with computerized software.

A method called Chip Assay uses PCR amplification of genomic segments by standard or long PCR protocols. Hybridization products are analyzed by VDA, Halushka et al., 1999, incorporated herein by reference. SNPs are generally classified as “Certain” or “Likely” based on computer analysis of hybridization patterns. By comparison to alternative detection methods such as nucleotide sequencing, “Certain” SNPs have been confirmed 100% of the time; and “Likely” SNPs have been confirmed 73% of the time by this method.

Other methods simply involve PCR amplification following digestion with the relevant restriction enzyme. Yet others involve sequencing of purified PCR products from known genomic regions.

In yet another method, individual exons or overlapping fragments of large exons are PCR-amplified. Primers are designed from published or database sequences and PCR-amplification of genomic DNA is performed using the following conditions: 200 ng DNA template, 0.5 *M each primer, 80 *M each of dCTP, dATP, dTTP and dGTP, 5% formamide, 1.5 mM MgCl2, 0.5U of Taq polymerase and 0.1 volume of the Taq buffer. Thermal cycling is performed and resulting PCR-products are analyzed by PCR-single strand conformation polymorphism (PCR-SSCP) analysis, under a variety of conditions, e.g, 5 or 10% polyacrylamide gel with 15% urea, with or without 5% glycerol. Electrophoresis is performed overnight. PCR-products that show mobility shifts are reamplified and sequenced to identify nucleotide variation.

In a method called CGAP-GAI (DEMIGLACE), sequence and alignment data (from a PHRAP.ace file), quality scores for the sequence base calls (from PHRED quality files), distance information (from PHYLIP dnadist and neighbour programs) and base-calling data (from PHRED ‘-d’ switch) are loaded into memory. Sequences are aligned and examined for each vertical chunk (‘slice’) of the resulting assembly for disagreement. Any such slice is considered a candidate SNP (DEMIGLACE). A number of filters are used by DEMIGLACE to eliminate slices that are not likely to represent true polymorphisms. These include filters that: (i) exclude sequences in any given slice from SNP consideration where neighboring sequence quality scores drop 40% or more; (ii) exclude calls in which peak amplitude is below the fifteenth percentile of all base calls for that nucleotide type; (iii) disqualify regions of a sequence having a high number of disagreements with the consensus from participating in SNP calculations; (iv) removed from consideration any base call with an alternative call in which the peak takes up 25% or more of the area of the called peak; (v) exclude variations that occur in only one read direction. PHRED quality scores were converted into probability-of-error values for each nucleotide in the slice. Standard Baysian methods are used to calculate the posterior probability that there is evidence of nucleotide heterogeneity at a given location.

In a method called CU-RDF (RESEQ), PCR amplification is performed from DNA isolated from blood using specific primers for each SNP, and after typical cleanup protocols to remove unused primers and free nucleotides, direct sequencing using the same or nested primers.

In a method called DEBNICK (METHOD-B), a comparative analysis of clustered EST sequencesis performed and confirmed by fluorescent-based DNA sequencing. In a related method, called DEBNICK (METHOD-C), comparative analysis of clustered EST sequences with phred quality>20 at the site of the mismatch, average phred quality>=20 over 5 bases 5′-FLANK and 3′to the SNP, no mismatches in 5 bases 5′ and 3′ to the SNP, at least two occurrences of each allele is performed and confirmed by examining traces.

In a method identified by ERO (RESEQ), new primers sets are designed for electronically published STSs and used to amplify DNA from 10 different mouse strains. The amplification product from each strain is then gel purified and sequenced using a standard dideoxy, cycle sequencing technique with ³³P-labeled terminators. All the ddATP terminated reactions are then loaded in adjacent lanes of a sequencing gel followed by all of the ddGTP reactions and so on. SNPs are identified by visually scanning the radiographs.

In another method identified as ERO (RESEQ-HT), new primers sets are designed for electronically published murine DNA sequences and used to amplify DNA from 10 different mouse strains. The amplification product from each strain is prepared for sequencing by treating with Exonuclease I and Shrimp Alkaline Phosphatase. Sequencing is performed using ABI Prism Big Dye Terminator Ready Reaction Kit (Perkin-Elmer) and sequence samples are run on the 3700 DNA Analyzer (96 Capillary Sequencer).

FGU-CBT (SCA2-SNP) identifies a method where the region containing the SNP is PCR amplified using the primers SCA2-FP3 (5′ CTCCGCCTCAGACTGTTTTGGTAG 3′) and SCA2-RP3 (5′ GTGGCCGAGGACGAGGAGAC 3′). Approximately 100 ng of genomic DNA is amplified in a 50 ml reaction volume containing a final concentration of 5 mM Tris, 25 mM KCl, 0.75 mM MgCl2, 0.05% gelatin, 20 pmol of each primer and 0.5U of Taq DNA polymerase. Samples are denatured, annealed and extended and the PCR product is purified from band cut out of the agarose gel using, for example, the QlAquick gel extraction kit (Qiagen) and is sequenced using dye terminator chemistry on an ABI Prism 377 automated DNA sequencer with the PCR primers.

In a method identified as JBLACK (SEQ/RESTRICT), two independent PCR reactions are performed with genomic DNA. Products from the first reaction are analyzed by sequencing, indicating a unique FspI restriction site. The mutation is confirmed in the product of the second PCR reaction by digesting with Fsp I.

In a method described as KWOK(1), SNPs are identified by comparing high quality genomic sequence data from four randomly chosen individuals by direct DNA sequencing of PCR products with dye-terminator chemistry (see Kwok et al., 1996). In a related method identified as KWOK (2) SNPs) are identified by comparing high quality genomic sequence data from overlapping large-insert clones such as bacterial artificial chromosomes (BACs) or P1-based artificial chromosomes (PACs). An STS containing this SNP is then developed and the existence of the SNP in various populations is confirmed by pooled DNA sequencing (see Taillon-Miller et al., 1998). In another similar method called KWOK(3), SNPs are identified by comparing high quality genomic sequence data from overlapping large-insert clones BACs or PACs. The SNPs found by this approach represent DNA sequence variations between the two donor chromosomes but the allele frequencies in the general population have not yet been determined. In method KWOK(5), SNPs are identified by comparing high quality genomic sequence data from a homozygous DNA sample and one or more pooled DNA samples by direct DNA sequencing of PCR products with dye-terminator chemistry. The STSs used are developed from sequence data found in publicly available databases. Specifically, these STSs are amplified by PCR against a complete hydatidiform mole (CHM) that has been shown to be homozygous at all loci and a pool of DNA samples from 80 CEPH parents (see Kwok et al., 1994).

In another such method, KWOK (OverlapSnpDetectionWithPolyBayes), SNPs are discovered by automated computer analysis of overlapping regions of large-insert human genomic clone sequences. For data acquisition, clone sequences are obtained directly from large-scale sequencing centers. This is necessary because base quality sequences are not present/available through GenBank. Raw data processing involves analyzed of clone sequences and accompanying base quality information for consistency. Finished (‘base perfect’, error rate lower than 1 in 10,000 bp) sequences with no associated base quality sequences are assigned a uniform base quality value of 40 (1 in 10,000 bp error rate). Draft sequences without base quality values are rejected. Processed sequences are entered into a local database. A version of each sequence with known human repeats masked is also stored. Repeat masking is performed with the program “MASKERAID.” Overlap detection: Putative overlaps are detected with the program “WUBLAST.” Several filtering steps followed in order to eliminate false overlap detection results, i.e. similarities between a pair of clone sequences that arise due to sequence duplication as opposed to true overlap. Total length of overlap, overall percent similarity, number of sequence differences between nucleotides with high base quality value “high-quality mismatches.” Results are also compared to results of restriction fragment mapping of genomic clones at Washington University Genome Sequencing Center, finisher's reports on overlaps, and results of the sequence conig building effort at the NCBI. SNP detection: Overlapping pairs of clone sequence are analyzed for candidate SNP sites with the ‘POLYBAYES’ SNP detection software. Sequence differences between the pair of sequences are scored for the probability of representing true sequence variation as opposed to sequencing error. This process requires the presence of base quality values for both sequences. High-scoring candidates are extracted. The search is restricted to substitution-type single base pair variations. Confidence score of candidate SNP is computed by the POLYBAYES software.

In method identified by KWOK (TaqMan assay), the TaqMan assay is used to determine genotypes for 90 random individuals. In method identified by KYUGEN(Q1), DNA samples of indicated populations are pooled and analyzed by PLACE-SSCP. Peak heights of each allele in the pooled analysis are corrected by those in a heterozygote, and are subsequently used for calculation of allele frequencies. Allele frequencies higher than 10% are reliably quantified by this method. Allele frequency=0 (zero) means that the allele was found among individuals, but the corresponding peak is not seen in the examination of pool. Allele frequency=0-0.1 indicates that minor alleles are detected in the pool but the peaks are too low to reliably quantify.

In yet another method identified as KYUGEN (Method1), PCR products are post-labeled with fluorescent dyes and analyzed by an automated capillary electrophoresis system under SSCP conditions (PLACE-SSCP). Four or more individual DNAs are analyzed with or without two pooled DNA (Japanese pool and CEPH parents pool) in a series of experiments. Alleles are identified by visual inspection. Individual DNAs with different genotypes are sequenced and SNPs identified. Allele frequencies are estimated from peak heights in the pooled samples after correction of signal bias using peak heights in heterozygotes. For the PCR primers are tagged to have 5′-ATT or 5′-GTT at their ends for post-labeling of both strands. Samples of DNA (10 ng/ul) are amplified in reaction mixtures containing the buffer (10 mM Tris-HCl, pH 8.3 or 9.3, 50 mM KCl, 2.0 mM MgCl2), 0.25 *M of each primer, 200 *M of each dNTP, and 0.025 units/*l of Taq DNA polymerase premixed with anti-Taq antibody. The two strands of PCR products are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. For the SSCP: an aliquot of fluorescently labeled PCR products and TAMRA-labeled internal markers are added to deionized formamide, and denatured. Electrophoresis is performed in a capillary using an ABI Prism 310 Genetic Analyzer. Genescan softwares (P-E Biosystems) are used for data collection and data processing. DNA of individuals (two to eleven) including those who showed different genotypes on SSCP are subjected for direct sequencing using big-dye terminator chemistory, on ABI Prism 310 sequencers. Multiple sequence trace files obtained from ABI Prism 310 are processed and aligned by Phred/Phrap and viewed using Consed viewer. SNPs are identified by PolyPhred software and visual inspection.

In yet another method identified as KYUGEN (Method2), individuals with different genotypes are searched by denaturing HPLC (DHPLC) or PLACE-SSCP (Inazuka et al., 1997) and their sequences are determined to identify SNPs. PCR is performed with primers tagged with 5′-ATT or 5′-GTT at their ends for post-labeling of both strands. DHPLC analysis is carried out using the WAVE DNA fragment analysis system (Transgenomic). PCR products are injected into DNASep column, and separated under the conditions determined using WAVEMaker program (Transgenomic). The two strands of PCR products that are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. SSCP followed by electrophoresis is performed in a capillary using an ABI Prism 310 Genetic Analyzer. Genescan softwares (P-E Biosystems). DNA of individuals including those who showed different genotypes on DHPLC or SSCP are subjected for direct sequencing using big-dye terminator chemistory, on ABI Prism 310 sequencer. Multiple sequence trace files obtained from ABI Prism 310 are processed and aligned by Phred/Phrap and viewed using Consed viewer. SNPs are identified by PolyPhred software and visual inspection. Trace chromatogram data of EST sequences in Unigene are processed with PHRED. To identify likely SNPs, single base mismatches are reported from multiple sequence alignments produced by the programs PHRAP, BRO and POA for each Unigene cluster. BRO corrected possible misreported EST orientations, while POA identified and analyzed non-linear alignment structures indicative of gene mixing/chimeras that might produce spurious SNPs. Bayesian inference is used to weigh evidence for true polymorphism versus sequencing error, misalignment or ambiguity, misclustering or chimeric EST sequences, assessing data such as raw chromatogram height, sharpness, overlap and spacing; sequencing error rates; context-sensitivity; cDNA library origin, etc.

In method identified as MARSHFIELD(Method-B), overlapping human DNA sequences which contained putative insertion/deletion polymorphisms are identified through searches of public databases. PCR primers which flanked each polymorphic site are selected from the consensus sequences. Primers are used to amplify individual or pooled human genomic DNA. Resulting PCR products are resolved on a denaturing polyacrylamide gel and a Phosphorlmager is used to estimate allele frequencies from DNA pools.

10. Methods of Nucleic Acid Transfer

For some methods of the present invention, methods of nucleic acid transfer may be employed. Suitable methods for nucleic acid delivery to effect expression of compositions of the present invention are believed to include virtually any method by which a nucleic acid (e.g., DNA, including viral and nonviral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Patients and Treatment

All patients had metastatic renal cancer. Laboratory criteria for adequate liver and kidney function were total bilirubin≦1.5 mg/dl, transaminases≦2.5×upper limit of normal, and creatinine≦2 mg/dl (or estimated creatinine clearance≧60 ml/min). Written informed consent was obtained from each patient in accordance with institutional and Federal guidelines.

The drug was supplied by the National Cancer Institute and administered every two weeks as a 72 h-continuous infusion at the dose of 50 mg/m2/day, the recommended phase II dose without diarrheal prophylaxis. Flavopiridol was reconstituted in benzyl alcohol preserved saline at a final concentration of 0.25 to 2.25 mg/ml in PVC bags and administered via an ambulatory pump. Toxicity assessment was done according to the Cancer and Leukemia Group B (CALGB) expanded toxicity criteria. For grade 3 or greater diarrheal episodes, patients were given loperamide or diphenoxylate hydrochloride plus atropine sulfate (Lomotil®). If diarrhea continued, treatment with octreotide acetate was begun. Any grade 3 and 4 toxicity led to a 25% and 50% dose reduction in subsequent cycles, respectively.

Assay Method and Pharmacokinetics

Heparinized blood samples were drawn at 23, 47, and 71 h into the infusion. Plasma was separated immediately by centrifugation and stored at −70° C. until analysis. Chemicals were obtained from Fisher Scientific (Itasca, Ill.) and Sigma Chemical Co. (St. Louis, Mo.). Quantitation of flavopiridol and its glucuronide in plasma was performed by high-pressure liquid chromatography (HPLC). One ml of plasma was combined with 20 μl of internal standard (flavone, 33.8 μM) and 5 ml of acetonitrile. After shaking at low speed for 10 min, samples were centrifuged at 2,500 rpm for 15 min (4° C.). The supernatant was evaporated to dryness using nitrogen gas (37° C.). After adding 500 μl of 0.1 M sodium phosphate buffer (pH 6.4) and 5 ml of acetonitrile, the samples were shaken (10 min) and centrifuged (15 min, 2,500 rpm, 4° C.). The samples were then dried down, reconstituted in 300 μl of mobile phase, and aliquots of 150 μl were injected into the HPLC system. Since flavopiridol glucuronide for calibration standards was not available in our laboratory, the estimation of flavopiridol glucuronide concentrations was performed by extracting plasma samples as described above with the only difference that the 0.1 M sodium phosphate buffer contained 2,500 units of β-glucuronidase enzyme (Sigma Chemical Co., St. Louis, Mo.), and these samples were incubated for 2 h (25° C.) prior to the addition of acetonitrile. Flavopiridol glucuronide concentrations were determined as the increase in flavopiridol levels after hydrolysis of flavopiridol glucuronide by β-glucuronidase.

The concentrations of flavopiridol were determined using an HPLC system (Hitachi Instruments, San Jose, Calif.) with UV detection at 269 nm. Flavopiridol and the internal standard were separated using a reversed-phase μBondapak Phenyl column (10 μm, 3.9×300 mm, Waters Corp., Milford, Mass.) preceded by a μBondapak Phenyl guardpak (Waters Corp., Milford, Mass.). The mobile phase consisted of 35% acetonitrile and 65% 50 mM ammonium acetate containing 1.46 g/l triethylamine (pH adjusted to 4.15 with 17.4 N acetic acid). At a flow of 1 ml/min, the retention times of flavopiridol and flavone were 9 and 21 min, respectively. Standard curves for flavopiridol in plasma were linear within the range of 37.3-3,953.5 nM. Human plasma for the preparation of calibration standards was purchased from the Blood Bank at the University of Chicago Hospitals. The inter-day and intra-day variability in the accuracy over the range of plasma concentrations investigated ranged from 1.0-7.9%, and 1.6-7.6%, respectively. The inter-day and intra-day variability in the precision (expressed as the coefficient of variation) was between 2.6-8.4%, and 0.7-8.4%, respectively. The extraction recovery at three different concentrations within the linear range of the calibration curve was 99.0±1.5 (mean±standard deviation).

Plasma samples for pharmacokinetics were collected during the first cycle of treatment (at 23, 47, and 71 h). Metabolic ratios for flavopiridol glucuronidation were calculated as the ratio of flavopiridol glucuronide to parent drug. Total clearance of flavopiridol at 23, 47, and 71 h was calculated by dividing the dosing rate by the drug concentration at each time point. The median clearance value of each patient was used to calculate the median clearance of flavopiridol among patients.

Statistical Analysis

Data are expressed as median values and interquartile range (the range of values between the 25th and the 75th percentile of the distribution). Significance has been tested by two-sided statistical tests. The Wilcoxon signed-rank test was used to compare the concentrations of flavopiridol, flavopiridol glucuronide and metabolic ratios at 23, 47, and 71 h during the infusion. For this analysis, the adjusted P<0.017 (0.05/3) was considered significant. Otherwise, a P value of <0.05 was considered the cut-off of significance. Plasma concentrations of flavopiridol and its glucuronide, and metabolic ratios were compared in patients with and without diarrhea by means of Mann-Whitney test. Moreover, Fisher's exact test was used to assess the association of flavopiridol systemic metabolism with the development of diarrhea. The distribution of the metabolic ratios at 71 h was investigated by constructing a frequency distribution histogram.

Results

Patient Characteristics and Demographics

Twenty-two patients (10 men and 12 women) with metastatic or unresectable renal carcinoma were evaluated for pharmacokinetics. The median age of patients was 58 years (56-63). Two of 22 patients were minorities (one each Hispanic and Black). Thirteen patients underwent prior nephrectomy. All but one patient had a CALGB performance status of 0-1, and all met the laboratory criteria for adequate liver and kidney function.

Pharmacokinetics and Systemic Metabolism of Flavopiridol

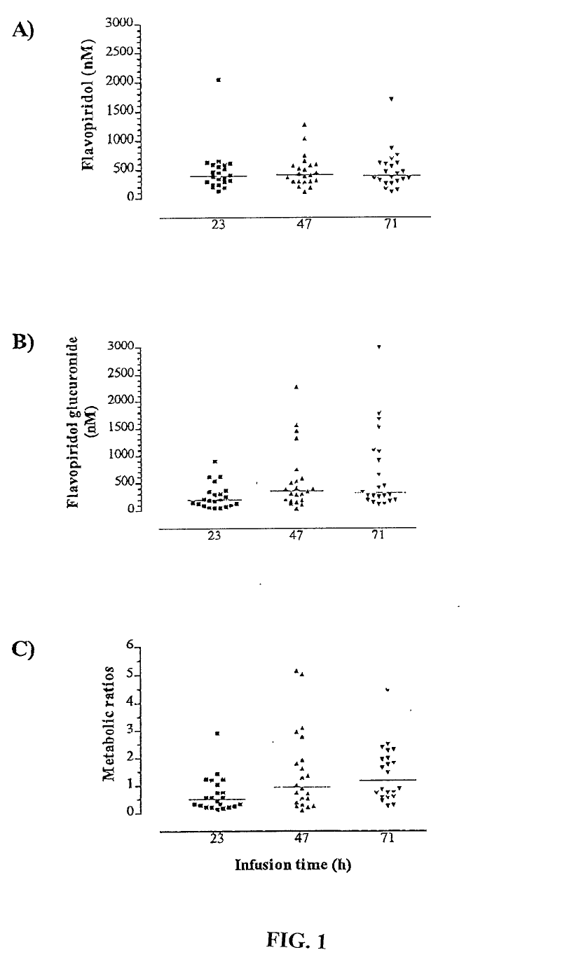

A steady-state level was apparently attained after 23 h of infusion, with flavopiridol median concentrations of 389 (296-567), 412 (297-566), and 397 (303-597) nM at 23, 47, and 71 h, respectively (FIG. 1, A). No significant difference was observed between the three collection times. Median total clearance of flavopiridol was 12.6 (9.7-17.0).

The pharmacokinetics of flavopiridol glucuronide show that the metabolite accumulated from 23 to 47 h into the infusion, reaching a plateau from 47 to 71 h. Plasma levels increased significantly from 194 (118-321) nM at 23 h to 358 (196-553) nM at 47 h (P<0.0001). At 71 h, the median flavopiridol glucuronide was 308 (218-990) nM. This value was lower than the median concentration at 47 h (P=0.04; FIG. 1, B). No clear correlation was observed between flavopiridol and its glucuronide concentrations (data not shown).

Metabolic ratio levels during the infusion reflected the increasing concentrations of flavopiridol glucuronide from 23 to 47 h and the constant levels of the parent drug during the infusion. A median value of 0.49 (0.27-0.9) at 23 h was significantly lower than values of 0.96 (0.44-1.87, P=0.0002) and 1.18 (0.65-1.96, P=0.0003) at 47 h, respectively (FIG. 1, C). Metabolic ratios at 47 and 71 h were not significantly different (P=0.35).

Maximal inter-patient variability for flavopiridol was observed at 23 h (coefficient of variation of 81%). Flavopiridol glucuronide levels were more variable, with coefficient of variations of 83, 102, and 107% at 23, 47, and 71 h, respectively. The inter-patient variability in metabolic ratios was 93, 99, and 72% at the corresponding sampling times.

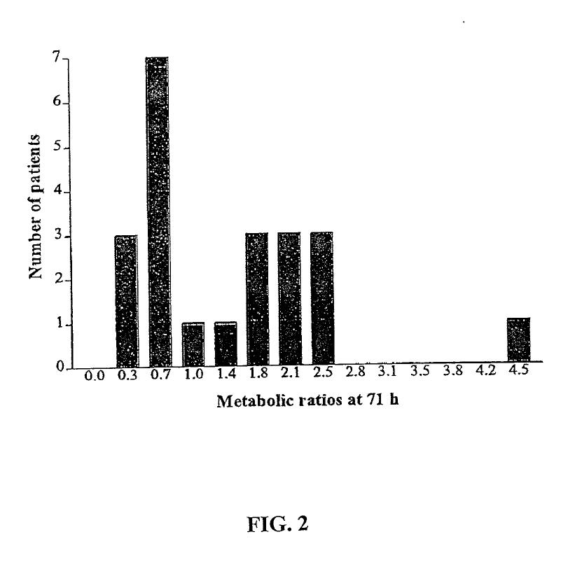

The frequency distribution histogram of the metabolic ratios at 71 h appeared to be bimodal, indicating the presence of two different groups of patients (FIG. 2). By adopting an antimode value of 1.2, they were tentatively categorized in “poor” and “extensive” glucuronidators of flavopiridol. Poor and extensive glucuronidators had metabolic ratios lower and higher than 1.2, respectively.

Association Between Flavopiridol Metabolism and Diarrhea

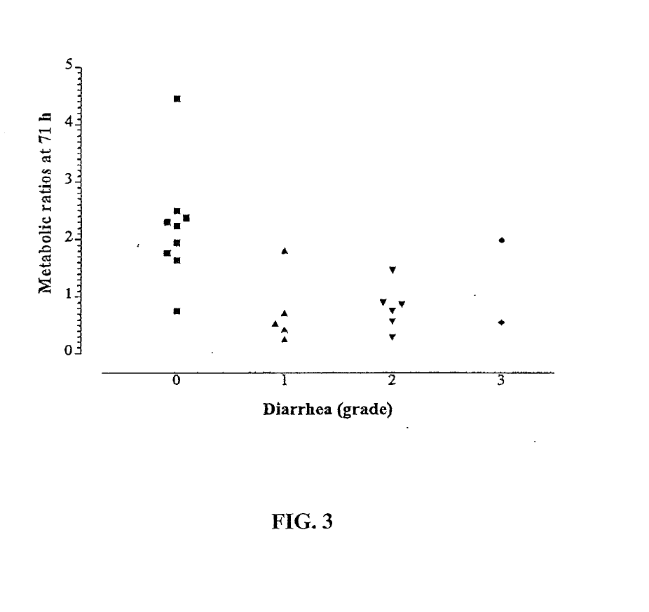

Thirteen of 22 patients experienced grade 1-3 diarrhea during the first cycle. Five patients had grade 1 diarrhea, six grade 2, and two grade 3. The typical onset of this toxicity was during the last day of the infusion, becoming less intense in the following 2-3 days. Patients who developed diarrhea had lower metabolic ratios at 71 h than patients without intestinal toxicity, with median metabolic ratios of 0.72 (0.53-0.86) and 2.24 (1.76-2.3), respectively (P=0.002). A less significant correlation was observed between metabolic ratios at 47 h (P=0.006). Diarrhea was not correlated with other pharmacokinetic parameters, such as flavopiridol and flavopiridol glucuronide levels during the infusion, and metabolic ratios at 23 h. The presence or absence of diarrhea was significantly associated with metabolic ratios at 71 h lower and higher than 1.2, respectively (P=0.008, Fisher's exact test). Moreover, 8 of 11 extensive glucuronidators did not develop diarrhea, while 10 of 11 poor glucuronidators developed diarrhea (FIG. 3).

### Example 2

Hepatic Metabolism and Glucuronidation of FLAVO

As FLAVO glucuronidation in cancer patients represents a crucial metabolic pathway, the present inventors investigated the in vitro formation of FLAVO glucuronide (FLAVO-G) using human liver microsomes. FLAVO-G was identified as 7-O-β-glucopyranuronosyl-flavopiridol using electron absorption electroscopy. Jager et al. (1998) reported that a complex is formed between Al³⁺ and a free hydroxyl group in position 5, causing a bathochromic shift in the absorption bands of 7-O-β-glucopyranuronosyl-flavopiridol. The same shift was observed after combining FLAVO-G and aluminum chloride, and it was determined that glucuronidation of FLAVO occurred at the 7-position. This is in agreement with previous experiments which show that in rat bile the metabolite 7-O-β-glucopyranuronosyl-flavopiridol is formed at a much higher rate than the 5-O glucuronide (Jager et al., 1998). Studies from Boutin et al. (1993) also show that the 7-position is the main conjugation site for the flavonoid acacetin, which is, like FLAVO, a 7,5-dihydroxy compound. The rate of FLAVO-G formation was examined using normal human and rat liver microsomes, as well as liver microsomes deficient in UGT1A activity. Microsomes expressing specific UGT isoforms belonging to both UGT1A and UGT2B families were screened to determine the enzyme(s) responsible for FLAVO glucuronidation.

Materials And Methods

Chemicals. FLAVO was provided by the Drug Synthesis and Chemistry Branch, Developmental Therapeutics Program, Division of Cancer Treatment and Diagnosis, National Cancer Institute (Bethesda, Md., USA). Acetonitrile (HPLC grade), aluminum chloride, ammonium acetate, dimethyl sulfoxide, glacial acetic acid, hydrochloric acid, methanol, potassium phosphate monobasic, sodium chloride, sodium phosphate monobasic, and sodium phosphate dibasic were obtained from Fisher Scientific (Itasca, Ill., USA). Flavone, propofol, p-fluorophenol, eugenol, 3,4-dimethoxystyrene, dithiothreitol, magnesium chloride, phosphoric acid, triethylamine, trizma base, uridine 5′-diphosphoglucuronic acid (UDPGA), and β-glucuronidase enzyme (E. coli type IX-A) were purchased from Sigma-Aldrich Co. (St. Louis, Mo., USA). The protein assay day reagent was obtained from Bio-Rad Laboratories (Hercules, Calif., USA).

Human liver samples. Normal human liver specimens were obtained following approval of Institutional Review Boards of the institutions involved through the Liver Tissue Procurement and Distribution System (National Institutes of Diabetes, Digestive and Kidney Diseases/NIDDK NO1-DK6-2274). The Crigler-Najjar type I (CN-I) liver specimen was kindly provided by Dr. R. K. Verbeeck at the Catholic University of Louvain, Belgium.

Preparation of microsomes from human and rat livers. Liver microsomes from normal humans (n=30), a CN-I patient (n=1), a normal rat (n=1; male, Harlan Sprague Dawley, Inc., Indianapolis, Ind., USA), and Gunn rats (n=3, male, Harlan Sprague Dawley, Inc., Indianapolis, Ind., USA) were prepared using differential centrifugation methods (Purba et al., 1987). Total protein content was determined by the Bradford method (Bradford, 1976) using bovine serum albumin as standard.

cDNA expressed UGT enzymes. Microsomes from human lymphoblasts expressing UGT1A1, UGT1A3, UGT1A4, UGT1A6, UGT1A9 and UGT2B15 enzymes, along with appropriate control (microsomes for lymphoblasts with vector alone) were obtained from Gentest Corporation (Woburn, Mass., USA). Microsomes containing baculovirus expressed UGT1A7 and UGT2B7 enzyme, and insect control microsomes were purchased from PanVera Corporation (Madison, Wis., USA). Incubation conditions were those adopted for human liver microsomes.

HK293 cell systems expressing UGT1A8, UGT1A10 and UGT2B4*D. HK293 cells were grown as previously reported (King et al., 1996; Levesque et al., 1999). Membranes from HK293 cells expressing UGT1A8 and UGT1A10 were prepared according to the method described by King et al. (1996). UGT2B4*D homogenates were prepared by resuspending the pelleted cells with 10 mM Tris buffered-saline (pH 7.4) containing 0.5 mM dithiothreitol. Cells were freezed-thawed three times and homogenized using a Wheaton tissue grinder (Wheaton Science Products, Milville, N.J.). The concentration of proteins in homogenized samples was measured by the Bradford method (Bradford, 1976). Incubation conditions were those adopted for human liver microsomes.

FLAVO glucuronidation assay. Reaction conditions were optimized using a range of substrate (0.02-1 mM), human liver microsomal protein (0.25-6 mg/ml) and UDPGA (2-10 mM) concentrations, as well as varying detergents (Brij 58, OLPC, CHAPS, Triton X-100, OLPC), and incubation times (0.5-4 h). The use of detergents did not affect enzyme activation (data not shown). The final incubation volume (250 μl) contained 50 mM tris-HCl (pH 7.4), 10 mM magnesium chloride, 0.5 mM FLAVO, 3 mg/ml protein, and 3 mM UDPGA. After incubation in a shaking water bath at 37° C. for 3 h, the reaction was terminated by the addition of 1 ml of acetonitrile. Precipitated proteins were removed by centrifugation (15 min, 11,000 rpm, 4° C.). The supernatant was spiked with 100 μl of internal standard (100 μg/ml flavone in acetonitrile), and evaporated to dryness using nitrogen gas. Control experiments were performed simultaneously by omitting UDPGA. The samples were reconstituted with 250 μl of 80% 50 mM ammonium acetate containing 0.1% (v/v) triethylamine (pH adjusted to 4.15 using glacial acetic acid) and 20% acetonitrile. Aliquots (125 μl) were analyzed by high performance liquid chromatography (HPLC; Hitachi Instruments, Inc., San Jose, Calif., USA) with UV detection (265 nm). Reversed-phase chromatography was carried out using a μBondapak Phenyl column (10 μm, 3.9×300 mm; Waters Corporation, Milford, Mass., USA), and a μBondapak Phenyl guard-pak insert (Waters Corporation, Milford, Mass., USA). The mobile phase consisted of 50 mM ammonium acetate containing 0.1% (v/v) triethylamine (pH 4.15; A) and acetonitrile (B). Elution was performed at a flow rate of 2 ml/min using the following gradient: 0-5 min, 80% A and 20% B; 5-15 min, 10% A and 90% B; 15-30 min, 80% A and 20% B. Since FLAVO-G was not available in our laboratory, its formation was expressed as the ratio of FLAVO-G to flavone peak heights. For determining the kinetic constants Kₘ and Vₘₐₓ, a pool of human liver microsomes was incubated varying substrate concentrations from 0.1 to 3 mM.

Propofol glucuronidation assay. Optimal reaction conditions were determined varying incubation times (0.25-3 h), and using a range of substrate (0.118-2 mM), protein (0.1-4 mg/ml) and UDPGA (1-10 mM) concentrations. The final incubation volume (250 μl) contained 0.2 M tris-HCl (pH 7.4), 10 mM magnesium chloride, 0.2 mM propofol, 1 mg/ml microsomal protein, and 1 mM UDPGA. The reaction was stopped after 30 min (37° C., shaking water bath) by adding 200 μl of methanol to the incubation. After protein removal by centrifugation (15 min, 14,000 rpm, 4° C.), the supernatant was combined with 20 μl of internal standard (530 ng/ml p-fluorophenol in methanol). Aliquots of 100 μl were analyzed by HPLC (Hitachi Instruments, Inc., San Jose, Calif., USA) with fluorescence detection (λ_{excitation}=276 nm, λₑₘᵢₛₛᵢₒₙ=310 nm). Control experiments were performed simultaneously by omitting UDPGA. Elution was performed using a μBondapak™ C₁₈ column (5 μm, 3.9×300 mm; Waters Corporation, Milford, Mass., USA) and a μBondapak™ C₁₈ guard-pak insert (Waters Corporation, Milford, Mass., USA). A mix of acetonitrile, deionized water and acetic acid (35/65/0.1, v/v/v) was used as mobile phase. The flow rate was 2 ml/min from 0-15 min, and 2.5 ml/min from 15.1-60 min. Under these conditions, p-fluorophenol, propofol glucuronide and propofol eluted at 4.0, 6.2, and 47.0 min, respectively. Since propofol glucuronide was not available in our laboratory, we expressed propofol glucuronidation activity as the ratio of propofol glucuronide to p-fluorophenol peak heights.

Eugenol glucuronidation assay. Eugenol was used a positive control for testing UGT2B4*D activity. Proteins (150 μg) were incubated in 50 mM Tris-HCl (pH 7.4) with 1 mM eugenol, 10 mM magnesium chloride and 5 mM UDPGA in a final volume of 100 μl. After 1 hr of incubation at 37° C. in a shaking water bath, the reaction was stopped with 100 μl of methanol followed by centrifugation (15 min, 14,000 rpm, 4° C.). The supernatant was then combined with 20 μl of internal standard (4 mM 3,4-dimethoxysterene), and evaporated to dryness using nitrogen gas (37° C.). The dry residue was dissolved in 200 μl of mobile, and 15 μl were injected into the HPLC with UV (220 nm) detection. Separation was achieved with a Novapak^{R} C₁₈ column (4 μm, 3.9×300 mm; Waters Corporation, Milford, Mass., USA) and a Novapak^{R} C₁₈ guard-pak insert (Waters Corporation, Milford, Mass., USA). A mobile phase of 5 mM potassium phosphate monobasic, acetonitrile and methanol (9/5.2/0.8, v/v/v) at pH 2 was used at a flow rate of 1 ml/min. The retention times for eugenol glucuronide, eugenol and 3,4-dimethoxystere were 1.9, 13.7 and 16.7 min, respectively.

Identification of FLAVO-G. Formation of FLAVO-G was confirmed by hydrolysis with β-glucuronidase enzyme. The incubation reaction was performed as described above. After centrifugation, the supernatant was treated with 2,500 units of β-glucuronidase enzyme dissolved in 500 μl of 0.1 M sodium phosphate buffer (pH 6.4). After 24 h, the sample was spiked with internal standard, dried, reconstituted as described above, and analyzed by HPLC. A control sample incubated with buffer alone was analyzed simultaneously.

Identification of FLAVO-G was performed using electron absorption spectroscopy. Spectrophotometric scans of FLAVO, FLAVO-G, and FLAVO-G were obtained after the addition of aluminum chloride (500 μM).

Data analysis and statistics. Results were expressed as mean ± standard deviation (SD) of a single experiment performed in triplicate. Lineweaver-Burke analysis of the data was used to calculate the enzyme kinetic parameters for the FLAVO glucuronidation reaction. The Spearman correlation coefficient was used to test the level of correlation between the formation of FLAVO and propofol glucuronides. The threshold value for statistical significance was set at 0.05. Data analysis and statistics were done using using GraphPad software (GraphPad Software Inc., San Diego, Calif.).

Results

Formation of FLAVO-G From FLAVO, and Identification of FLAVO-G

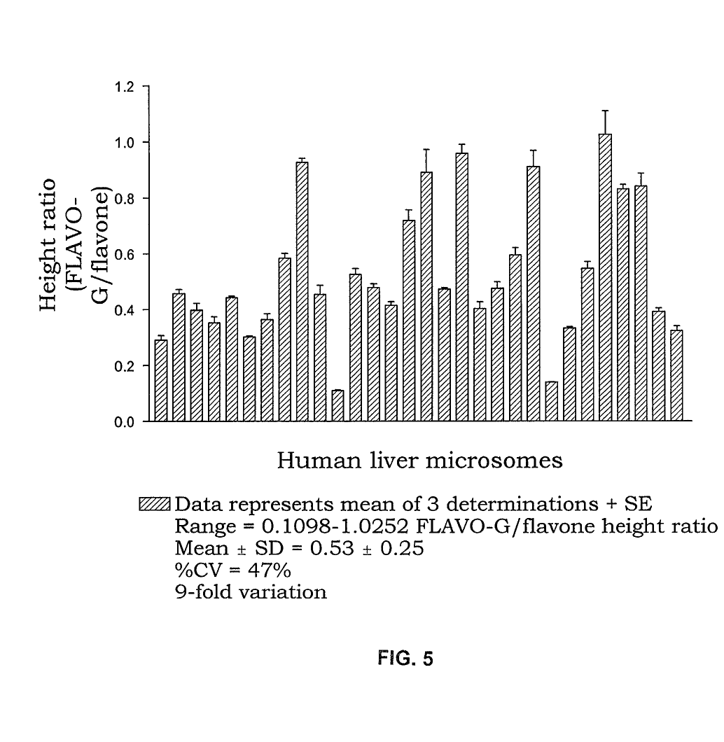

FIG. 5A illustrates the chromatogram obtained after in vitro incubation of FLAVO with normal human liver microsomes. Retention times for FLAVO-G, FLAVO and flavone were 6.5, 11.5 and 13.5 min, respectively. The estimated Km and Vmax for glucuronidation of FLAVO by human liver microsomes were 0.6421 mM and 1.067 FLAVO-G/flavone peak height ratio, respectively.

Treatment with β-glucuronidase enzyme confirmed the formation of FLAVO-G since enzyme addition to human liver microsomal incubations caused the disappearance of FLAVO-G, and the corresponding increase in FLAVO. When a control sample was analyzed simultaneously with buffer (no β-glucuronidase added), the peak of FLAVO-G remained intact. Further confirmation was provided by the absence of FLAVO-G formation when UDPGA was omitted from the incubation medium. To determine the position at which glucuronidation occurs, the FLAVO-G peak was collected and combined with aluminum chloride. The bathochromic shift in the absorption spectra of FLAVO-G revealed that the metabolite formed was due to the conjugation of UDPGA with the alcohol group in the C-7 of FLAVO (7-O-β-glucopyranuronosyl-flavopiridol; Jager et al., 1998).

Inter-individual Variability of FLAVO Glucuronidation in Human Liver Microsomes

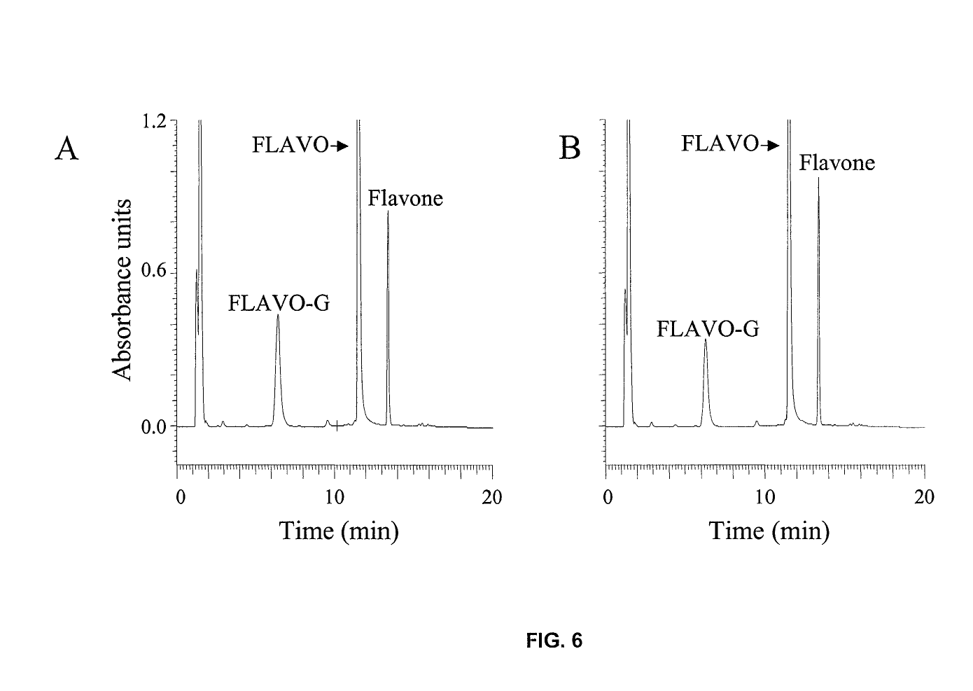

Inter-individual variation in the capacity to glucuronidate FLAVO was evaluated on 30 human liver microsomes (FIG. 6). The FLAVO-G/flavone peak height ratio varied 9-fold, ranging from 0.11-1.03. The mean value (±S.D.) was 0.53±0.25. The coefficient of variation was 47%.

Glucuronidation of FLAVO in Human Liver Microsomes from a CN-I Patient

FIG. 5B illustrates the chromatograms obtained after incubation of FLAVO with liver microsomes from a CN-I patient. Microsomes from a CN-I patient had glucuronidation activity of 0.59±0.01.

Glucuronidation of FLAVO by cDNA Expressed Isoforms of UGT

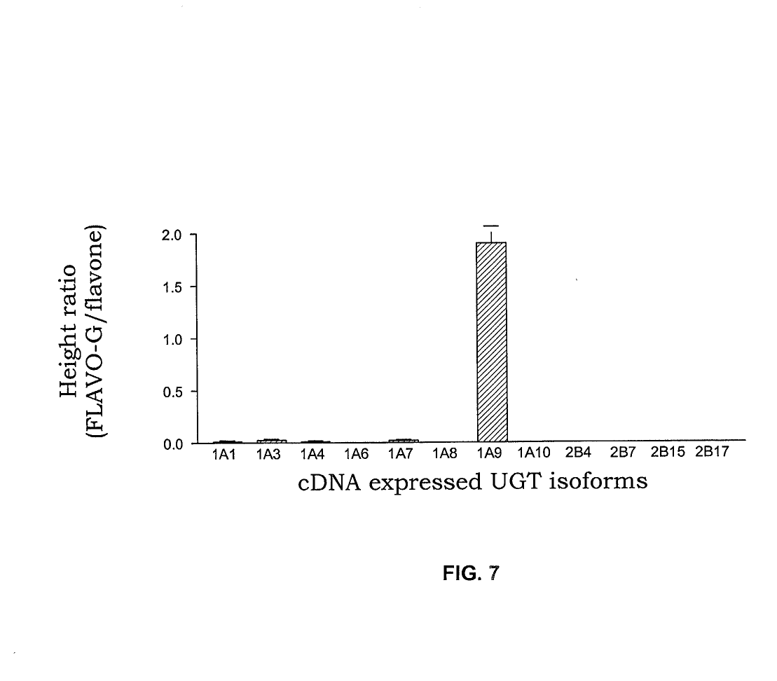

To determine the particular UGT enzyme(s) involved in the glucuronidation of FLAVO, eleven individual human UGT isoforms were screened for FLAVO activity. UGT1A9 was the main isoform involved in the reaction. UGT1A1, UGT1A3, UGT1A4, and UGT1A7 displayed only 0.5, 1.6, 0.5, and 1.1% of the activity exhibited by UGT1A9, respectively (FIG. 7). FLAVO was not a substrate of the isoforms UGT1A6, UGT1A8, UGT1A10, UGT2B4, UGT2B7, and UGT2B15.

Glucuronidation of Propofol by cDNA Expressed Isoforms of UGT

Propofol glucuronidation was assayed using nine single enzyme isoforms. The enzymes UGT1A9, UGT1A7, UGT2B15 and UGT2B7 had propofol glucuronidating activity of 0.41±0.00, 0.08±0.03, 0.07±0.00 and 0.05±0.01, respectively. UGT1A1, UGTA3, UGT1A4, UGT1A6 and UGT1A10 did not catalyze the formation of propofol glucuronide.

Correlation Studies With Propofol

A low to moderate correlation was found between the in vitro glucuronidation of FLAVO and propofol (Spearrnan's correlation coefficient r=0.36, p=0.048). Propofol glucuronidating activity assessed in 30 human liver microsomes varied 3-fold, ranging from 0.20-0.66 (mean±S.D.=0.50±0.12, CV=24%).

Discussion

Screening of microsomes from one particular CN-I patient revealed glucuronidation activity in the mid range of activities observed in normal human liver microsomes. CN-I patients have absent UGT1Al activity (de Wildt et al., 1999). These patients could also have mutations in the conserved region of the UGT1 gene complex causing the loss of UGT1A activity (Iyanagi et al., 1998).

### Example 3

Pharmacogenetic Screening and Polymorphism Analysis

Data from the in vitro incubations and the clinical trials show considerable variability in the capacity to glucuronidate FLAVO, and an apparent bimodality in the formation of FLAVO-G. The clinical studies showed that the extent of systemic FLAVO glucuronidation was inversely related to the incidence of diarrhea. It is contemplated that this variability reflects genetic differences in the isozymes catalyzing the formation of FLAVO-G. Thus, the inventors contemplate that pharmacogenetic screening prior to administration of FLAVO can identify individuals predisposed to FLAVO drug toxicity. FLAVO drug toxicity based on the presence of a polymorphic enzyme that may catalyze the formation of FLAVO-G may also explain the unpredictable pharmacokinetics of FLAVO.

All of the COMPOSITIONS and METHODS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS and METHODS and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.

Abigerges et. al., J Clin Oncol, 13(1):210-221, 1995.

Abraham et al., Cancer Chemother. Pharmacol., 32(2): 116-122, 1993.

Akiyama et al., Mol. Pharmacol., 33(2):144-147, 1988.

Ansher et al., Hepatology, 3(6):932-935, 1983.

Araki et al., Jpn. J. Cancer Res., 84:697-702, 1993.

Ariyoshi et al, Jpn. J. Cancer Res., 83(5):515-521, 1992.

Atsumi et al, Xenobiotica, 21:1159-1169, 1991.

Barilero et al., J. Chrom., 575:275-280, 1992.

Bear, Biochem. Biophys. Res. Commun., 200(1):513-521, 1994.

Bell et al., Biochem. Pharmacol., 48(3):495-503, 1994.

Benderra, et al. Adv Exp Med Biol ;457:151-60, 1999.

Bible, et al., Cancer Res 1997;57:3375-80

Bock et al., Eur. J. Biochem., 98:19-26, 1979.

Bock et. al., In: Conjugation reactions in biotransformation, Elsevier, North Holland Biomedical Press, p. 357-364, 1978.

Boesch & Loor, Anticancer Drugs, 5(2):229-238, 1994.

Boesch et al., Exp. Cell. Res., 196(1):26-32, 1991.

Boiteux-Antoine et al., Gen-Pharmacol, 20(4):407-412, 1989.

Borrel et al., Eur. J Biochem., 223(1):125-133, 1994.

Burchell and Coughtrie, Pharmac. Ther., 43:261-289, 1989.

Burger et al., Antimicromal Agents and Chemotherapy, 37(7):1426-1431, 1993.

Burris & Fields, Hematol Oncol Clin North Am, 8(2):333-355, 1994

Burris et al., J. Natl. Cancer Inst., 84:1816-1819, 1992.

Campain et al., J. Cell Physiol., 155(2):414-425, 1993.

Carlson, et al., Cancer Res 1996;56:2973-8

Cascorbi et al., Clinc. Pharmacol Ther, 69(3):169-74, 2001.

Chabot et al, J. Chromatogr, 575:275, 1992

Charuk et al., A. Am. J. Physiol., 35:F66-F75, 1994.

Chien, et al., Cancer Chemother Pharmacol 1999;44:81-7

Chin et al., J Cell Physiol., 152(1):87-94, 1992.

Coffman, et al. Drug Metab Dispos, 1998;26:73-7

Curtin, et al. Eur J Cancer. 35(6):1020-6, 1999.

Czech, et al., Int J Oncol 1995;6:31-6

D'Arpa & Liu, Biochim et. Biophys. Acta, 989:163-177, 1989.

Davies & Schnell, Toxicology and Applied Pharmacology, 109:39-40, 1991.

De Morais & Wells, Hepatology, 10:163-167, 1989.

De Morais et al., Toxicology and Applied Pharmacology, 117:81-87, 1992.

Decleves et al., 2000, Human Mutation, 15(5): 486

Di Carlo, et al. Life Sci 1999;65:337-53

Diasio, et al., Clin Pharmacokinet 1989;16:215-37

Doige et al., Biochim. Biophys. Acta., 1109(2):149-160, 1992.

Draper, et al. Br J Cancer;75(6):810-5, 1997.

Drees et al., Clin Cancer Res ;3:273-9, 1997.

Duffy, et al. Eur J Cancer, 34(8): 1250-9, 1998.

Egner et al., Carcinogenesis, 15(2):177-181, 1994.

Ejima et. al., Chem Pharm Bull, 40(3):683-688, 1992.

Emerson et. al., Cancer Res, 55(3):603-609, 1995

Endicott & Ling, Annus Re. Biochem., 58:137-171, 1989.

Ford et al., Cancer Res., 50(6): 1748-1756, 1990.

Fournel et al., Biochimica at Biophysica Acta, 842:202-213, 1985.

Foxwell et al., Mol. Pharmacol., 36:543-546, 1989.

Frank, et al. J Cancer Res Clin Oncol 125(2):117-20, 1999.

Friche et al., Br. J. Cancer, 67(2):226-231, 1993.

Friche et. al., Biochem. Pharmacol., 39, 1721-1726; 1990

Friedman et al., Cancer Chemother. Pharmacol., 34:171-174, 1994.

Furuta et al., Jpn. J. Cancer Chemother., 15:2757-2760, 1988.

Giovanella et al., Cancer Res., 51:3052-3055, 1991.

Giovanella et al, Science, 246:1046-1048, 1989.

Gottlieb et al., Cancer Chemother. Rep., 54:461-470, 1970.

Gruol et al., Cancer Res., 54(12):3088-3091, 1994.

Gupta et al., Cancer res., 54:3723-3725, 1994b.

Gupta et al., Pharm. Res., 11 :S450, 1994a.

Gupta, et al., Cancer Res 1994;54:3723-5

Gutmann et al. Drug Metab Dispos. 27(8):937-41, 1999.

Hait et al., Biochem. Pharmacol., 45(2):401-406, 1993.

Hamada et al., Cancer Res., 50(11):3167-3171, 1990.

Harris et al., Cancer, 68:499-501, 1991.

Hawkins, Oncology, 6(12):17-23, 1992

Hecht et al., Proceedings of the American Association for Cancer Research, 35, 1994

Hirohashi, et al. J Pharmacol Exp Ther 292(1):265-70, 2000

Hjelle, J. Pharmacol. Exp. Ther., 237:750-756, 1986.

Hoffineyer et al., 2000, PNAS, 28:97(7), 3473-3478.

Hooijberg et al., 1999, British J of Cancer, 81: 2, 269-276.

Horwitz and Horwitz, Cancer Res., 33:2834-2836, 1973.

Hsiang et al., J. Biol. Chem., 27:14873-14878, 1985.

Hunter et al, Pharm. Res., 10(5):743-749, 1993.

Ichikawa-Haraguchi et al, Biochim. Biophys. Acta, 1158(3):201-208, 1993.

Inoue et al., J. Biol. Chem., 268(8):5894-5898, 1993.

Ishizuka et al. J Pharmacol Exp Ther. 287(1):37-42 1998

Ito et al., 2001, Pharmacogenetics, 11: 2, 175-184.

Iyer, et al. Eur J Cancer 1998;1493-9

Iyer, Mol Diagnosis 1999;4:327-33

Jager, et al., Life Sci 1998;62:1861-73

Kamimoto et al., J. Biol. Chem., 264:11693-11698, 1989.

Kamiwatari et al, Cancer Res., 49(12):3190-3195, 1989.

Kaneda & Yokokura, Cancer Res., 50:1721-1725, 1990.

Kaneda et al., Cancer Res., 50:1715-1720, 1990.

Kang, et al. Cancer Res, 1;54(11):2952-8, 1994

Kaplan et al., J. Pharm. Biopharm., 1:201-216, 1973.

Kaur, et al., J Natl Cancer Inst 1992;84: 1736-40

Kawato et al., Cancer Res., 51:4187-4191, 1991.

Ken-Ichi et al., 2001, J. Biological Chem., Feb 21, 2001.

Kerb et al., 2001, Pharmacogenomics, 2:1, 51-64.

Kingsbury et al., J Med Chem, 34(1):98-107, 1991.

Kiue et al., Cancer Res., 50(2):310-317, 1990.

Klein, et al. Bioch Biophys Acta, 1461, 237-262 (1999).

Kunimoto et al., Cancer Res., 47:5944-5947, 1987.

Kusuhara, et al. Am J Physiol., 275(4 Pt 1):G789-961998

Lannoy et al., Am. J. Physiol., 263(4 Pt 2):F613-622, 1992.

Lennard et al., Clin. Pharmacol. Ther., 46:149-154, 1989.

Lennard, Eur J Clin Pharmacol 1992;43:329-39

Levesque, et al., Pharmacogenetics 1997;7:317-25

Levesque, et al, Pharmacogenetics 1999;9:207-16

Li et al., Cancer Res., 32:2643-2650, 1970.

Ling, Cancer Chemother Pharmacol;40 Suppl:S3-8,1997

Lornri, et al. Semin. Liv. Dis., 16(2): 201-210, 1996.

Losiewicz, et al., Biochem Biophys Res Commun 1994;201 :589-95

Lubet et al., Biochem. Pharmacol., 43(5):1067-1078, 1992.

Magdalou et al, Biol. Cell., 77(1):13-16, 1993.

Manning and Franklin, Toxicology, 65(1-2):149-159, 1990.

Mattem et. al., Oncol Res, 5(12):467-474, 1993.

Mazzanti et al., Hepatology, 20(1 Pt 1): 170-176, 1994.

McKinney and Hosford, J. Biol. Chem., 268(10):6886-6895, 1993.

Mechetner & Roninson, Proc. Natl. Acad. Sci. USA, 89(13):5824-5828, 1992.

Meech. et al Arch Biochem Biophys 1998;356:77-85

Miki & Kotake, Hinyokika Kiyo, 39(12):1221-1225, 1993.

Miller et al., J. Clin. Oncol., 9(1):17-24, 1991.

Miners, et al., Pharmacol Ther 1991;51:347-69

Miyamoto et al, Anticancer Res., 12(3):649-653, 1992b.

Miyamoto et al., Cancer Lett., 64(2):177-183, 1992a.

Miyamoto et al., Cancer Res., 53(7):1555-1559, 1993.

Morris et al, Biochemistry, 30(34):8371-8379, 1991.

Muggia et al., Cancer Chemother. Rep., 56:515-521, 1972.

Muller et al., FEBS Lett., 343(2): 168-172, 1994.

Nebert, Clin Gen 1999;56:247-58

Negoro et al., J. Natl. Cancer Inst., 83:1164-1168, 1991.

Niwa et al., Cancer Res., 52(13):3655-3660, 1992.

Ohe et al., J. Natl Cancer Inst., 84:972-974, 1992.

Ohi et al, Cancer Chemother Pharmacol, 30(Suppl):S50-S54, 1992.

Okamura et al., J. Pharmacol. Exp. Therap., 266:1614-1619, 1993.

Pang et al. Hepatology. 28(5):1341-6, 1998

Patel et al., Pharmacogenetics, 2:38-45, 1992.

Payen, et al., Biochem Biophys Res Commun, 19;258(3):513-8, 1999.

Perdu and Germain, 2001, Human Mutation, 17:1, 74-75.

Pourtier-Manzanedo et al., Oncol. Res., 4(11-12):473-480, 1992.

Prochaska and Femandes, Carcinogenesis, 14(12):2441-2443, 1993.

Rajaonarison et. al, Drug Metab. Disp., 19:809-815, 1993.

Ramirez et al.,. Clin Pharmacol Ther 1998;63:149 (abstract)

Ratain et al., Clin. Pharmacol. Ther., 50:573-579, 1991.

Ratain, et al., Clin Pharmacol Ther 1991;50:573-9

Robey et al., 2001, Clinical Cancer Res., 7: 1, 145-152.

Rothenberg et al., J. Clin. Oncol., 11:2194-2204, 1993.

Rowinsky et al., Cancer Res., 54:427-436, 1994.

Rund et al, 1999, Adv. Exp Med Biol., 457:71-75.

Saeki et al., J. Biol. Chem., 268(9):6077-6080, 1993.

Sakamoto et al. Cancer Lett. 8;135(1):113-9, 1999.

Sausville, et al., Pharmacol Ther 1999;82:285-92

Savas, et al. Anticancer Res 19(5C):4413-20 1999

Schinkel et al., Cell, 77(4):491-502, 1994.

Schinkel et al., Int. J. Cancer, 55(3):478-484, 1993.

Schrump, et al., Clin Cancer Res 1998;4:2885-90

Sedlacek, et al., Int J Oncol 1996;9:1143-68

Senderowicz, et al, J Clin Oncol 1998;16:2986-99

Shapiro, et al., Clin Cancer Res 1999;5:2925-38

Shenfeld et al Gastroenterology 1997;112:A404 (abstract)

Sherr, Science 1996;274:1672-7

Shirai et al, Biochim. Biophys. Acta, 1222(3):400-404, 1994.

Silber et. al., Blood, 84(10):3440-3446, 1994.

Slichenmyer et. al., Cancer Chemother Pharmacol, 34(Suppl):S53-57, 1994.

Slichenmyer et. al., J Natl Cancer Inst, 85(4):271-291, 1993.

Stadler, et al., J Clin Oncol 2000; 18:371-5

Sugasawa et. al, J Med Chem, 19(5):675-679, 1976.

Sugimori et. al., J Med Chem, 37(19):3033-3039, 1994.

Suzuki, et al Semin Liver Dis ;18(4):359-76, 1998

Suzuki, Gan To Kagaku Ryoho, 17(3 Pt 1):335-341, 1990.

Takiguchi et. al., Gan To Kagaku Ryoho, 21(5):705-708, 1994.

Tamai & Safa, J. Biol. Chem., 266:16796-16800, 1991.

Tasaki, et. al., J Urol 154(3):1210-6, 1995

Tatsuta et al., Anticancer Drug Des., 6(3):179-188, 1991.

Thalhammer et al, Eur. J. Pharmacol., 270(2-3):213-220, 1994.

Thiebaut et al., Proc. Natl. Acad. Sci. USA, 84:7735-7738, 1987.

Thomas et al., Proc Am Assoc Cancer Res 1997;38:A 1496 (abstract)

Trump et al., J. Natl. Cancer Inst., 84(23):1811-6, 1992.

Tsuji et al., J. Pharmacobio-Dyn., 14:341-349, 1991.

Tsuruo et al., Cancer Chemother. Pharmacol., 21:71-74, 1988.

Vezmar, et al. Biochem Pharmacol;59(10):1245-1252

Wall et al., J Am. Chem. Soc., 88:3888-3890, 1966.

Wall et. al., J. Med Chem, 36(18):2689-2700, 1993.

Wani et al., J Med. Chem., 23:554-560, 1980.

Wani et. al., J. Med Chem, 30(10):1774-1779, 1987.

Wilson et al, Biochem. Biophys. Res. Commun., 176(3):1377-1382, 1991.

Worland, et al., Biochem Pharmacol 1993 ;46:1831-40

Zacher et al., Cancer Chemother. Pharmacol., 34(2):125-132, 1994.

Zalups, J Toxicol Environ Health. 55(1):13-29, 1998

Zhang, et al. Drug Metab Dispos., 28(3):329-34, 2000.

What is claimed is:

1. A method for reducing the toxicity of flavopiridol or an analog thereof, comprising administering said flavopiridol, or analog thereof, to an animal in combination with an amount of a second agent effective to reduce excretion of an active flavopiridol species through the bile.

2. The method of claim 1, wherein said second agent increases flavopiridol catabolism.

3. The method of claim 2, wherein said second agent increases glucuronosyltransferase enzyme activity.

4. The method of claim 3, wherein said glucuronyltransferase enzyme is selected from the group including uridine 5′diphosphate glucoronyltansferase (UGT)1A1, UGT1A3, UGT1A4, UGT1A6, UGT1A8, UGT1A10, UGT2B4, UGT2B7, UGT2B15 and UGT2B17.

5. The method of claim 3, wherein said second agent is a dithiolethione.

6. The method of claim 3, wherein said second agent is Oltipraz.

7. The method of claim 3, wherein said second agent is an aryloxycarboxylic acid, an arylcarboxylic acid, a chlorophenoxycarboxylic acid or a fibric acid.

8. The method of claim 7, wherein said second agent is clofibrate, ciprofibrate, fenofibrate, bezafibrate, gemfibrazol, tiadenol or probucol.

9. The method of claim 3, wherein said second agent is phenobarbital, dilantin, clonazepam, clotrimazole, buthionine sulfoximine (BSO), cyclophosphamide, ifosphamide, a retinoic acid, rifampin or disulfiram (Antabuse).

10. The method of claim 3, wherein said second agent is a corticosteroid.

11. The method of claim 3, wherein said second agent is an oral contraceptive.

12. The method of claim 1, wherein said second agent decreases biliary transport.

13. The method of claim 12, wherein said second agent decreases the activity of an ABC protein.

14. The method of claim 13, wherein said ABC protein is a biliary transport protein.

15. The method of claim 14, wherein said biliary transport protein is p-glycoprotein.

16. The method of claim 14, wherein said wherein said biliary transport protein is bile salt export protein.

17. The method of claim 14, wherein said wherein said biliary transport protein is MDR3.

18. The method of claim 14, wherein said ABC protein is a multi-drug resistance-associated protein (MRP).

19. The method of claim 18, wherein said multi-drug resistance-associated protein is cMOAT.

20. The method of claim 18, wherein said multi-drug resistance-associated protein is MRP1.

21. The method of claim 18, wherein said multi-drug resistance-associated protein is MRP5.

22. The method of claim 12, wherein said second agent is a cyclosporine, cephalosporin or a staurosporine.

23. The method of claim 22 wherein said second agent is SDZ 280 446, 3′-Keto-cyclosporin D, cefoperazone or staurosporine.

24. The method of claim 22, wherein said second agent is Cyclosporine A, SDZ PSC 833 (valspodar) or NA-382.

25. The method of claim 12, wherein said second agent is a calcium channel blocker.

26. The method of claim 25, wherein said second agent is a dihydropyridine analogue, verapamil, dex verapamil, tiapamil, nifedipine, diltiazem, nicardipine, nisoldipine, nimodipine or nitrendipine.

27. The method of claim 12, wherein said second agent is a calmodulin antagonist.

28. The method of claim 27, wherein said second agent is trans-flupenthixol, cis-flupenthixol, clorpenthixol, fluphenazine, chlorpromazine, triflupromazine, trifluoperazine, prochlorperazine or thioridazine.

29. The method of claim 12, wherein said second agent is progesterone, a progesterone metabolite, pregnenolone, RU 486 or tirilazad.

30. The method of claim 12, wherein said second agent is reserpine, dipyridamole, chloroquine, propranolol, terfenadine, ivermectin or quinidine.

31. The method of claim 12, wherein said second agent is an antibody that binds one or more biliary transporter proteins.

32. The method of claim 12, wherein said second agent is a cytokine.

33. The method of claim 32, wherein said cytokine is interferon-β.

34. The method of claim 12, wherein said second agent is ritonavir

35. The method of claim 12, wherein said second agent is MK571.

36. The method of claim 12, wherein said second agent is genistein.

37. The method of claim 12, wherein said second agent is a reduced folate.

38. The method of claim 12, wherein said second agent is probenecid.

39. The method of claim 12, wherein said second agent is a non-steroidal anti-inflammatory drug.

40. The method of claim 39, wherein said non-steroidal anti-inflammatory drugs is selected from the group including indomethacin, sulindac, tolmetin, acemetacin, zomepirac, mefenamic acid, ibuprofen, ketoprofen, piroxicam, naproxen, sulindac, aspirin, choline subsalicylate, diflunisal, fenoprofen, meclofenamate, salsalate, tolmetin, etodolac, nabumetone, oxaprozin, rofecoxib, celocoxib, diflunisal, salsalate, ketorolac, tolectin, clinoril, mefenamic acid, fenoprofen calcium, meclofenamate sodium, piroxicam, diclofenac and magnesium salicylate.

41. The method of claim 1, comprising administering said flavopiridol compound in combination with a second agent that increases conjugative enzyme activity and a third agent that decreases biliary transport protein activity.

42. A method for treating an animal with cancer, comprising administering to said animal a therapeutically effective combination of a flavopiridol drug and a second agent that reduces excretion of the active flavopiridol species through the bile.

43. The method of claim 42 wherein said second agent is administered to the animal prior to said flavopiridol drug.

44. The method of claim 42 wherein said flavopiridol drug or said second agent are administered parenterally.

45. The method of claim 42 wherein said flavopiridol drug or said second agent are administered orally.

46. The method of claim 42 wherein said animal is a human patient.

47. The method of claim 42, wherein said second agent increases the activity of a conjugative enzyme or decreases the activity of a biliary transport protein.

48. The method of claim 42 wherein said second agent increases glucuronosyltransferase enzyme activity.

49. The method of claim 42 wherein said second agent is selected from the group including Oltipraz, clofibrate, ciprofibrate, fenofibrate, bezafibrate, gemfibrazol, tiadenol, probucol, phenobarbital, dilantin, clonazepam, clotrimazole, buthionine sulfoximine (BSO), cyclophosphamide, ifosphamide, a retinoic acid, a corticosteroid, an oral contraceptive, rifampin and disulfiram (Antabuse).

50. The method of claim 42 wherein said second agent is phenobarbital, Oltipraz, all-trans retinoic acid, phenytoin, dexamethasone, rifampin or clofibrate.

51. The method of claim 47 wherein said second agent decreases biliary transport protein activity.

52. The method of claim 51, wherein said second agent is selected from the group including SDZ 280 446, 3′-Keto-cyclosporin D, cefoperazone, staurosporine, Cyclosporine A, SDZ PSC 833 (valspodar), NA-382, dihydropyridine analogue, verapamil, dex verapamil, tiapamil, nifedipine, diltiazem, nicardipine, nisoldipine, nimodipine, nitrendipine, trans-flupenthixol, cis-flupenthixol, clorpenthixol, fluphenazine, chlorpromazine, triflupromazine, trifluoperazine, prochlorperazine, thioridazine, progesterone, a progesterone metabolite, pregnenolone, RU 486, tirilazad, reserpine, dipyridamole, chloroquine, propranolol, terfenadine, ivernectin, quinidine [(3)H]2,4-Dinitrophenyl-S-glutathione (DNP-SG), [(3)H]17beta-estradiol 17-beta-D-glucuronide, dipyridamole (E(2)17betaG), 7-chloro-4-nitrobenz-2-oxa-1,3-diazole, buthionine sulphoximine, MK751, leukotriene C4 (LTC4), bromosulfophthalein (BSP), enalapril, CRC 220, taurocholate (TCA), N-acetylcysteine and cysteine, [3H]Temocaprilat, estradiol-17beta-D-glucuronide, dibromosulphophthalein, genistein, probenecid, ritonavir, indomethacin, sulindac, tolmetin, acemetacin, zomepirac, mefenamic acid, ibuprofen, ketoprofen, piroxicam, naproxen, sulindac, aspirin, choline subsalicylate, diflunisal, fenoprofen, meclofenamate, salsalate, tolmetin, etodolac, nabumetone, oxaprozin, rofecoxib, celocoxib, diflunisal, salsalate, ketorolac, tolectin, clinoril, mefenamic acid, fenoprofen calcium, meclofenamate sodium, piroxicam, diclofenac magnesium salicylate and interferon α.

53. The method of claim 42, comprising administering a therapeutically effective combination of flavopiridol drug, a second agent that increases conjugative enzyme activity and a third agent that decreases biliary transport protein activity.

54. The method of claim 42, comprising administering about 25-100 mg/m²/day flavopiridol by infuision over about 90 minutes, about 90 mg of phenobarbital and about 5 mg/kg cyclosporine A by infuision.

55. A composition comprising a flavopiridol drug in combination with a second agent that increases conjugative enzyme activity or that decreases biliary transport protein activity.

56. The composition of claim 55, wherein said second agent increases glucuronosyltransferase enzyme activity.

57. The composition of claim 56, wherein said second agent is selected from the group including Oltipraz, clofibrate, ciprofibrate, fenofibrate, bezafibrate, gemfibrazol, tiadenol, probucol, phenobarbital, dilantin, clonazepam, clotrimazole, buthionine sulfoximine (BSO), cyclophosphamide, ifosphamide, a retinoic acid, a corticosteroid, an oral contraceptive, rifampin and disulfiram (Antabuse).

58. The composition of claim 57, wherein said second agent is phenobarbital, Oltipraz, all-trans retinoic acid, phenytoin, dexamethasone, rifampin or clofibrate.

59. The composition of claim 55, wherein said second agent decreases biliary transport protein activity.

60. The composition of claim 59, wherein said second agent is selected from the group comprising SDZ 280 446, 3′-Keto-cyclosporin D, cefoperazone, staurosporine, Cyclosporine A, SDZ PSC 833 (valspodar), NA-382, dihydropyridine analogue, verapamil, dex verapamil, tiapamil, nifedipine, diltiazem, nicardipine, nisoldipine, nimodipine, nitrendipine, trans-flupenthixol, cis-flupenthixol, clorpenthixol, fluphenazine, chlorpromazine, triflupromazine, trifluoperazine, prochlorperazine, thioridazine, progesterone, a progesterone metabolite, pregnenolone, RU 486, tirilazad, reserpine, dipyridamole, chloroquine, propranolol, terfenadine, ivermectin, quinidine [(3)H]2,4-Dinitrophenyl-S-glutathione (DNP-SG), [(3)H]17beta-estradiol 17-beta-D-glucuronide, dipyridamole (E(2)17betaG), 7-chloro-4-nitrobenz-2-oxa-1,3-diazole, buthionine sulphoximine, MK751, leukotriene C4 (LTC4), bromosulfophthalein (BSP), enalapril, CRC 220, taurocholate (TCA), N-acetylcysteine and cysteine, [3H]Temocaprilat, estradiol-17beta-D-glucuronide, dibromosulphophthalein, genistein, probenecid, interferon α, indomethacin, sulindac, tolmetin, acemetacin, zomepirac, mefenamic acid, ibuprofen, ketoprofen, piroxicam, naproxen, sulindac, aspirin, choline subsalicylate, diflunisal, fenoprofen, meclofenamate, salsalate, tolmetin, etodolac, nabumetone, oxaprozin, rofecoxib, celocoxib, diflunisal, salsalate, ketorolac, tolectin, clinoril, mefenamic acid, fenoprofen calcium, meclofenamate sodium, piroxicam, diclofenac magnesium salicylate.and ritonavir.

61. The composition of claim 55, dispersed in a pharmacologically acceptable formulation.

62. A therapeutic kit comprising, in suitable container means, a pharmaceutical formulation of a flavopiridol drug and a pharmaceutical formulation of a second agent that increases glucuronosyltransferase enzyme activity or that decreases biliary transport protein activity.

63. The kit of claim 62, wherein said flavopiridol drug and said second agent are present within a single container means.

64. The kit of claim 62, wherein said flavopiridol drug and said second agent are present within distinct container means.

65. The kit of claim 62, wherein said pharmaceutical formulation is suitable for parenteral or oral administration.

66. The kit of claim 62, wherein said second agent is phenobarbital, Oltipraz, all-trans retinoic acid, phenytoin, dexamethasone, rifampin or clofibrate.

67. The kit of claim 62, comprising flavopiridol, a pharmaceutical formulation of a second agent wherein said second agent is selected from the group comprising SDZ 280 446, 3′-Keto-cyclosporin D, cefoperazone, staurosporine, Cyclosporine A, SDZ PSC 833 (valspodar), NA-382, dihydropyridine analogue, verapamil, dex verapamil, tiapamil, nifedipine, diltiazem, nicardipine, nisoldipine, nimodipine, nitrendipine, trans-flupenthixol, cis-flupenthixol, clorpenthixol, fluphenazine, chlorpromazine, triflupromazine, trifluoperazine, prochlorperazine, thioridazine, progesterone, a progesterone metabolite, pregnenolone, RU 486, tirilazad, reserpine, dipyridamole, chloroquine, propranolol, terfenadine, ivermectin, quinidine [(3)H]2,4-Dinitrophenyl-S-glutathione (DNP-SG), [(3)H]17beta-estradiol 17-beta-D-glucuronide, dipyridamole (E(2)17betaG), 7-chloro-4-nitrobenz-2-oxa-1,3-diazole, buthionine sulphoximine, MK751, leukotriene C4 (LTC4), bromosulfophthalein (BSP), enalapril, CRC 220, taurocholate (TCA), N-acetylcysteine and cysteine, [3H]Temocaprilat, estradiol-17beta-D-glucuronide, dibromosulphophthalein, genistein, probenecid, interferon (α, indomethacin, sulindac, tolmetin, acemetacin, zomepirac, mefenamic acid, ibuprofen, ketoprofen, piroxicam, naproxen, sulindac, aspirin, choline subsalicylate, diflunisal, fenoprofen, meclofenamate, salsalate, tolmetin, etodolac, nabumetone, oxaprozin, rofecoxib, celocoxib, diflunisal, salsalate, ketorolac, tolectin, clinoril, mefenamic acid, fenoprofen calcium, meclofenamate sodium, piroxicam, diclofenac magnesium salicylate.and ritonavir.

68. A method for predicting the degree of a flavopiridol drug toxicity in a patient, comprising determining the glucuronidation or biliary transport capacity of the patient, wherein a decreased glucuronidation capacity or an increased biliary transport capacity, in comparison to normal levels, is indicative of a patient at risk of flavopiridol drug toxicity.

69. The method of claim 68, comprising determining the relevant genotype of the patient.

70. The method of claim 68, comprising determining the relevant phenotype of the patient.

71. The method of claim 68, comprising determining the glucuronidation capacity of the patient.

72. The method of claim 68, wherein the glucuronidation capacity of the patient is determined by administering a glucuronidatable substrate to said patient and determining the degree of glucuronidation of said substrate.

73. The method of claim 72, wherein said substrate of glucoronidation is acetaminophen, diflunisal or morphine.

74. A method for reducing the toxicity of a flavopiridol compound, comprising administering said flavopiridol compound to an animal in combination with an amount of Cyclosporine A effective to reduce excretion of an active flavopiridol species through the bile by decreasing the activity of a biliary transport protein.

75. The method of claim 3, wherein said glucuronyltransferase enzyme is a uridine 5′diphosphate glucoronyltansferase (UGT)1A1, UGT1A3, UGT1A4, UGT1A7, or UGT1A9.

76. The method of claim 75, wherein said glucuronyltransferase enzyme is UGT1A9.

77. A method for evaluating the risk of flavopiridol-induced toxicity in a patient comprising evaluating a UGT1A9-encoding nucleic acid of the patient for a polymorphism.

78. The method of claim 77, further comprising identifying a patient at risk for flavopiridol-induced toxicity.

79. The method of claim 77, further comprising identifying a polymorphism in a UGT1A9-encoding nucleic acid of the patient.

80. The method of claim 79, wherein the nucleic acid is encoded by SEQ. ID. NO. 1.

81. The method of claim 79, wherein the nucleic acid is encoded by SEQ. ID. NO. 3.

82. The method of claim 77, wherein the polymorphism results in a decreased level of UGT1A9 activity in the patient.

83. The method of claim 77, wherein the polymorphism results in a decreased level of UGT1A9 expression in the patient.

84. The method of claim 77, further comprising identifying a polymorphism in an ABC gene.

85. A method for reducing the toxicity of flavopiridol or an analog thereof in a cancer patient comprising
a) identifying a polymorphism in a UGT1A9 gene in a sample from the patient, wherein the polymorphism contributes to reduced expression or activity of the UGT1A9 gene product; and
b) administering to the patient a second agent effective to reduce excretion of an active flavopiridol species through the bile.

86. The method of claim 13, wherein the ABC protein is ABCA1, ABCA2, ABCA3, ABCA4, ABCA5, ABCA6, ABCA7, ABCA8, ABCA9, ABCA10, ABCA11, ABCA12, ABCA13, ABCA14, ABCB1, ABCB2, ABCB3, ABCB4, ABCB5, ABCB6, ABCB7, ABCB8, ABCB9, ABCB10, ABCB1, ABCC1, ABCC2, ABCC3, ABCC4, ABCC5, ABCC6, ABCC7, ABCC8, ABCC9, ABCC10, ABCC11, ABCC12, ABCC13, ABCD1, ABCD2, ABCD3, ABCD4, ABCE1, ABCF1, ABCF2, ABCF3, ABCG1, ABCG2, ABCG4, ABCG5, or ABCG8.

87. The method of claim 86, wherein the ABC protein is BCRP1.

88. A method for evaluating the risk of flavopiridol-induced toxicity in a patient comprising evaluating a ABC-encoding nucleic acid of the patient for a polymorphism.

89. The method of claim 88, further comprising identifying a patient at risk for flavopiridol-induced toxicity.

90. The method of claim 88, further comprising identifying a polymorphism in a ABC-encoding nucleic acid of the patient.

91. The method of claim 88, wherein the polymorphism results in a decreased level of ABC activity in the patient.

92. The method of claim 88, wherein the polymorphism results in a decreased level of ABC expression in the patient.

93. The method of claim 88, wherein the ABC-encoding nucleic acid encodes ABCA1, ABCA2, ABCA3, ABCA4, ABCA5, ABCA6, ABCA7, ABCA8, ABCA9, ABCA10, ABCA11, ABCA12, ABCA13, ABCA14, ABCB1, ABCB2, ABCB3, ABCB4, ABCB5, ABCB6, ABCB7, ABCB8, ABCB9, ABCB10, ABCB1, ABCC1, ABCC2, ABCC3, ABCC4, ABCC5, ABCC6, ABCC7, ABCC8, ABCC9, ABCC10, ABCC11, ABCC12, ABCC13, ABCD1, ABCD2, ABCD3, ABCD4, ABCE1, ABCF1, ABCF2, ABCF3, ABCG1, ABCG2, ABCG4, ABCG5, or ABCG8.

94. The method of claim 93, wherein the ABC-encoding nucleic acid encodes BCRP1.

95. The method of claim 93, wherein the ABC-encoding nucleic acid encodes MRP1.

96. The method of claim 93, wherein the ABC-encoding nucleic acid encodes MRP2.

97. The method of claim 93, wherein the ABC-encoding nucleic acid encodes PGY1.

98. A method for reducing the toxicity of flavopiridol or an analog thereof in a cancer patient comprising
a) identifying a polymorphism in a ABC gene in a sample from the patient, wherein the polymorphism contributes to reduced expression or activity of the ABC gene product; and
b) administering to the patient a second agent effective to reduce excretion of an active flavopiridol species through the bile.

99. The method of claim 98, wherein the second agent is a UGT1A9 protein.


SEQUENCE LISTING (4 sequences)

SEQ ID NO: 1 - DNA, 2320 bp, Homo sapiens
  CDS (19)..(1611)
ctcagctgcagttctctgatggcttgcacagggtggaccagcccccttcctctatgtgtgtgtctgctgctgacctgtggctttgccgaggcagggaagctactggtagtgcccatggatgggagccactggttcaccatgaggtcggtggtggagaaactcattctcagggggcatgaggtggttgtagtcatgccagaggtgagttggcaactgggaagatcactgaattgcacagtgaagacttattcaacttcatataccctggaggatctggaccgggagttcaaggcttttgcccatgctcaatggaaagcacaagtacgaagtatatattctctattaatgggttcatacaatgacatttttgacttatttttttcaaattgcaggagtttgtttaaagacaaaaaattagtagaatacttaaaggagagttcttttgatgcagtgtttctcgatccttttgataactgtggcttaattgttgccaaatatttctccctcccctccgtggtcttcgccaggggaatactttgccactatcttgaagaaggtgcacagtgccctgctcctctttcctatgtccccagaattctcttagggttctcagatgccatgactttcaaggagagagtacggaaccacatcatgcacttggaggaacatttattatgccaccgttttttcaaaaatgccctagaaatagcctctgaaattctccaaacacctgttacggagtatgatctctacagccacacatcaatttggttgttgcgaacggactttgttttggactatcccaaacccgtgatgcccaacatgatcttcattggtggtatcaactgccatcagggaaagccgttgcctatggaatttgaagcctacattaatgcttctggagaacatggaattgtggttttctctttgggatcaatggtctcagaaattccagagaagaaagctatggcaattgctgatgctttgggcaaaatccctcagacagtcctgtggcggtacactggaacccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacccattgagaagtgggtgggaaataaggtaaaattttgaaccattccctagtcatttccaaacttgaaaacagaatcagtgttaaattcattttattcttattaaggaaatactttgcataaattaatcagccccagagtgctttaaaaaattctcttaaataaaaataatagactcgctagtcagtaaagatatttgaatatgtatcgtgccccctccggtgtctttgatcaggatgacatgtgccatttttcagaggacgtgcagacaggctggcattctagattacttttcttactctgaaacatggcctgtttgggagtgcgggattcaaaggtggtcccaccgctgcccctactgcaaatggcagttttaatcttatcttttggcttctgcagatggttgcaattgatccttaaccaataatggtcagtcctcatctctgtcctgcttcataggtgccaccttgtgtgtttaaagaagggaagctttgtacctttagagtgtaggtgaaatgaatgaatggcttggagtgcactgagaacagcatatgatttcttgctttggggaaaaagaatgatgctatgaaattggtgggtggtgtatttgagaagataatcattgcttatgtcaaatggagctgaatttgataaaaacccaaaatacagctatgaagtgctgggcaagtttactttttttctgatgtttcctacaact

SEQ ID NO: 2 - protein, 530 aa, Homo sapiens
MACTGWTSPLPLCVCLLLTCGFAEAGKLLVVPMDGSHWFTMRSVVEKLILRGHEVVVVMPEVSWQLGRSLNCTVKTYSTSYTLEDLDREFKAFAHAQWKAQVRSIYSLLMGSYNDIFDLFFSNCRSLFKDKKLVEYLKESSFDAVFLDPFDNCGLIVAKYFSLPSVVFARGILCHYLEEGAQCPAPLSYVPRILLGFSDAMTFKERVRNHIMHLEEHLLCHRFFKNALEIASEILQTPVTEYDLYSHTSIWLLRTDFVLDYPKPVMPNMIFIGGINCHQGKPLPMEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 3 - DNA, 2320 bp, Homo sapiens; identical to SEQ ID NO: 1
  CDS (19)..(1611)

SEQ ID NO: 4 - protein, 530 aa, Homo sapiens; identical to SEQ ID NO: 2